(19) United States
(12) Patent Application Publication
(10) Pub. No.: US 2026/0231677 A1
(43) Pub. Date: Aug. 6, 2026

(54) NITROGEN-CONTAINING COMPOUND, ORGANIC ELECTROLUMINESCENT DEVICE AND ELECTRONIC APPARATUS

(71) Applicant: Shaanxi Lighte Optoelectronics Material Co., Ltd., Xi'an (CN)

(72) Inventors: Tiantian MA, Xi'an (CN); Heming ZHANG, Xi'an (CN)

(21) Appl. No.: 19/151,366

(22) PCT Filed: Sep. 2, 2024
(86) PCT No.: PCT/CN2024/116404

§ 371 Date: Jul. 28, 2025

(30) Foreign Application Priority Data
Oct. 24, 2023 (CN) 202311386681.5

(51) Int. Cl. H10K 85/60; C07B 59/00; C07D 209/88; C09K 11/02; H10K 50/11

(52) U.S. Cl. CPC H10K 85/6572; C07B 59/002; C07D 209/88; C09K 11/02; H10K 50/11

(57) ABSTRACT

The present application belongs to the technical field of organic electroluminescence, and relates to a nitrogen-containing compound, an organic electroluminescent device and an electronic device. The nitrogen-containing compound has a structure as represented by formula 1. When the nitrogen-containing compound is used in the organic electroluminescent device, the properties of the organic electroluminescent device can be significantly improved.

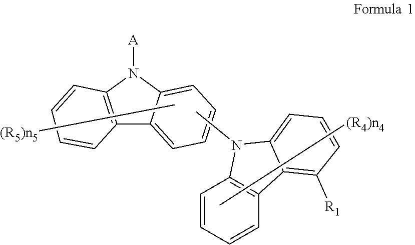

### CROSS REFERENCE TO RELATED APPLICATIONS

The present disclosure claims the priority of Chinese patent application No. 202311386681.5, filed on Oct. 24, 2023, the contents of which are incorporated here by reference in its entirety as part of the present disclosure.

### TECHNICAL FIELD

The present disclosure relates to the technical field of organic compounds, in particular to a nitrogen-containing compound, and an organic electroluminescent device and an electronic apparatus including the nitrogen-containing compound.

### BACKGROUND

With the development of electronic technology and the progress of material science, the application range of electronic elements for realizing electroluminescence is wider and wider. Such electronic element generally includes a cathode and an anode which are disposed oppositely, and a functional layer disposed between the cathode and the anode. The functional layer is composed of a plurality of organic or inorganic film layers and generally includes an organic light-emitting layer, a hole transport layer located between the organic light-emitting layer and the anode, and an electron transport layer located between the organic light-emitting layer and the cathode. Taking an organic electroluminescent device as an example, the organic electroluminescent device generally includes an anode, a hole transport layer, an electron blocking layer, an organic light-emitting layer, an electron transport layer and a cathode which are sequentially stacked. When voltage is applied to the cathode and the anode, an electric field is generated between the two electrodes, electrons on the cathode side move towards the organic light-emitting layer and holes on the anode side also move towards the organic light-emitting layer under the action of the electric field. The electrons and the holes are combined in the organic light-emitting layer to form excitons, and the excitons are in an excited state and release energy outwards, and then the organic light-emitting layer emits light outwards.

The prior art discloses host materials for the organic light-emitting layer in the organic electroluminescent device. However, it is still necessary to continue to develop novel materials so as to further improve the performance of the organic electroluminescent device.

### SUMMARY

In order to solve the above problems, an object of the present disclosure is to provide a nitrogen-containing compound, and an organic electroluminescent device and an electronic apparatus including the nitrogen-containing compound. The nitrogen-containing compound can improve the performance of the organic electroluminescent device and the electronic device, such as reducing the driving voltage of the device, improving the efficiency and service life of the device.

A first aspect of the present disclosure provides a nitrogen-containing compound, having a structure shown in a Formula 1:

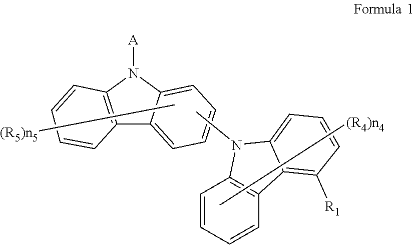

wherein, R₁ is selected from a hydrogen, a deuterium, an aryl with 6 to 12 carbon atoms or a deuteroaryl with 6 to 12 carbon atoms;
A is selected from groups represented by a Formula 1-1;

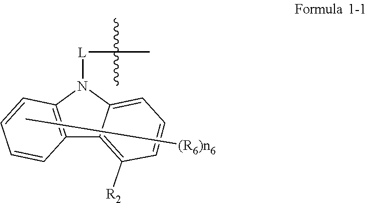

R₂ is selected from a hydrogen, a deuterium, an aryl with 6 to 12 carbon atoms or a deuteroaryl with 6 to 12 carbon atoms, and at least one of R₁ and R₂ is an aryl with 6 to 12 carbon atoms or a deuteroaryl with 6 to 12 carbon atoms;
or, A is selected from groups represented by a Formula 1-2;

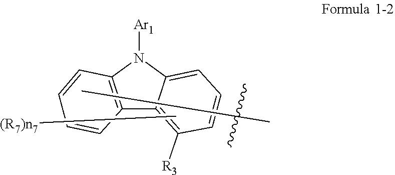

R₃ is selected from a hydrogen, a deuterium, an aryl with 6 to 12 carbon atoms or a deuteroaryl with 6 to 12 carbon atoms, and at least one of R₁ and R₃ is an aryl with 6 to 12 carbon atoms or a deuteroaryl with 6 to 12 carbon atoms;
each R₄, each R₅, each R₆ and each R₇ are the same or different, and are respectively and independently selected from a deuterium, an aryl with 6 to 12 carbon atoms or a deuteroaryl with 6 to 12 carbon atoms;
n₄ is the number of R₄, and is selected from 0, 1, 2, 3, 4, 5, 6 or 7, and when n₄ is greater than 1, each R₄ is the same or different;
n₅ is the number of R₅, and is selected from 0, 1, 2, 3, 4, 5, 6 or 7, and when n₅ is greater than 1, each R₅ is the same or different;
no is the number of R₆, and is selected from 0, 1, 2, 3, 4, 5, 6 or 7, and when no is greater than 1, each R₆ is the same or different;
n₇ is the number of R₇, and is selected from 0, 1, 2, 3, 4, 5 or 6, and when n₇ is greater than 1, each R₇ is the same or different;
L is selected from a substituted or unsubstituted arylene with 6 to 12 carbon atoms;
substituent(s) of L are the same or different, and are respectively and independently selected from a deuterium, a phenyl or a pentadeuterophenyl;
Ar₁ is selected from a substituted or unsubstituted aryl with 6 to 30 carbon atoms; and
substituent(s) of Ar₁ are the same or different, and are respectively and independently selected from a deuterium, a halogen group, a cyano, an alkyl with 1 to 10 carbon atoms, a cycloalkyl with 3 to 10 carbon atoms, an aryl with 6 to 12 carbon atoms, a deuteroaryl with 6 to 12 carbon atoms, or a haloaryl with 6 to 12 carbon atoms.

A second aspect of the present disclosure provides an organic electroluminescent device, including an anode and a cathode which are oppositely disposed, and a functional layer disposed between the anode and the cathode, where the functional layer includes the nitrogen-containing compound according to the first aspect of the present disclosure.

A third aspect of the present disclosure provides an electronic apparatus, including the organic electroluminescent device according to the second aspect.

The nitrogen-containing compound provided in the present disclosure uses triple carbazole in a specific connection manner as a core structure, and at least one of groups connected to a 4-position of carbazolyl located at both ends is aryl or deuteroaryl. The triple carbazole structure used in the present disclosure has excellent hole transport characteristics, while at least one of the groups connected at the 4-position of the carbazolyl at both ends is aryl or deuteroaryl, and the above structure can improve steric hindrance of the compound structure, and can improve the film-forming properties of a material. When the nitrogen-containing compound of the present disclosure is used as a host material of an organic light-emitting layer in an organic electroluminescent device, the carrier balance in the organic light-emitting layer can be improved, a carrier recombination region can be broadened, the exciton generation and utilization efficiency can be increased, the luminous efficiency of the device can be improved, and the service life of the device can be prolonged.

Other features and advantages of the present disclosure will be described in detail in the subsequent detailed description.

### BRIEF DESCRIPTION OF THE DRAWINGS

The accompanying drawings are used to provide a further understanding of the present disclosure and constitute a part of the description, and are used to explain the present disclosure together with the following specific embodiments, but do not constitute limitations on the present disclosure.

FIG. 1 is a schematic structural diagram of an organic electroluminescent device according to the present disclosure.

FIG. 2 is a schematic structural diagram of an electronic apparatus according to the present disclosure.

### REFERENCE SIGNS

100, anode; 200, cathode; 300, functional layer; 310, hole injection layer; 320, hole transport layer; 330, electron blocking layer; 340, organic light-emitting layer; 350, electron transport layer; 360, electron injection layer; 400, electronic apparatus.

### DETAILED DESCRIPTION

In view of the above problems existing in the prior art, an object of the present disclosure is to provide a nitrogen-containing compound, and an organic electroluminescent device including the nitrogen-containing compound, and an electronic device. The nitrogen-containing compound can improve the performance of the organic electroluminescent device and the electronic device, such as reducing the driving voltage of the device, and improving the efficiency and service life of the device.

A first aspect of the present disclosure provided is a nitrogen-containing compound, having a structure shown in a Formula 1:

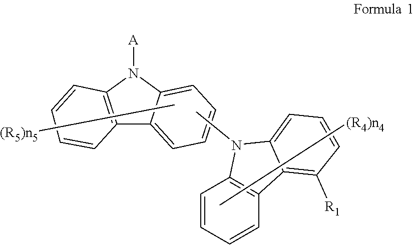

wherein, R₁ is selected from a hydrogen, a deuterium, an aryl with 6 to 12 carbon atoms or a deuteroaryl with 6 to 12 carbon atoms;
A is selected from groups represented by a Formula 1-1;

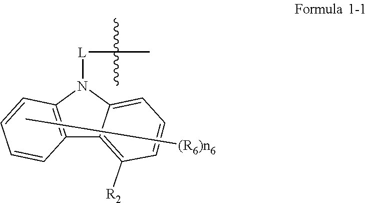

R₂ is selected from a hydrogen, a deuterium, an aryl with 6 to 12 carbon atoms or a deuteroaryl with 6 to 12 carbon atoms, and at least one of R₁ and R₂ is an aryl with 6 to 12 carbon atoms or a deuteroaryl with 6 to 12 carbon atoms;
or, A is selected from groups represented by a Formula 1-2;

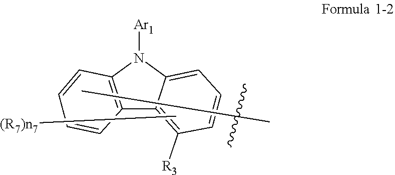

R₃ is selected from a hydrogen, a deuterium, an aryl with 6 to 12 carbon atoms or a deuteroaryl with 6 to 12 carbon atoms, and at least one of R₁ and R₃ is an aryl with 6 to 12 carbon atoms or a deuteroaryl with 6 to 12 carbon atoms;
each R₄, each R₅, each R₆ and each R₇ are the same or different, and are respectively and independently selected from a deuterium, an aryl with 6 to 12 carbon atoms, or a deuteroaryl with 6 to 12 carbon atoms;
n₄ is the number of R₄, and is selected from 0, 1, 2, 3, 4, 5, 6 or 7, and when n₄ is greater than 1, each R₄ is the same or different;
n₅ is the number of R₅, and is selected from 0, 1, 2, 3, 4, 5, 6 or 7, and when n₅ is greater than 1, each R₅ is the same or different;
no is the number of R₆, and is selected from 0, 1, 2, 3, 4, 5, 6 or 7, and when no is greater than 1, each R₆ is the same or different;
n₇ is the number of R₇, and is selected from 0, 1, 2, 3, 4, 5 or 6, and when n₇ is greater than 1, each R₇ is the same or different;
L is selected from a substituted or unsubstituted arylene with 6 to 12 carbon atoms;
substituent(s) of L are the same or different, and are respectively and independently selected from a deuterium, a phenyl or a pentadeuterophenyl;
Ar₁ is selected from a substituted or unsubstituted aryl with 6 to 30 carbon atoms; and
substituent(s) of Ar₁ are the same or different, and are respectively and independently selected from a deuterium, a halogen group, a cyano, an alkyl with 1 to 10 carbon atoms, a cycloalkyl with 3 to 10 carbon atoms, an aryl with 6 to 12 carbon atoms, a deuteroaryl with 6 to 12 carbon atoms, or a haloaryl with 6 to 12 carbon atoms.

In the present disclosure, the adopted description modes “each . . . is independently”, “ . . . is respectively and independently” and “ . . . is each independently” can be interchanged, and should be understood in a broad sense, which means that in different groups, specific options expressed between the same symbols do not influence each other, or in a same group, specific options expressed between the same symbols do not influence each other. For example, the meaning of

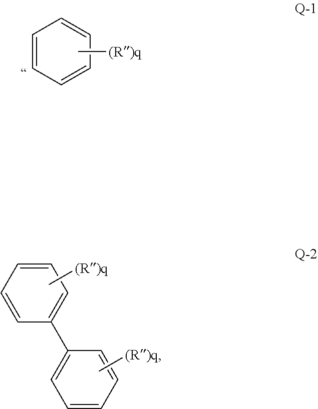

where each q is independently 0, 1, 2 or 3, and each R″ is independently selected from hydrogen, deuterium, fluorine and chlorine” is as follows: a formula Q-1 represents that q substituents R″ exist on a benzene ring, each R″ can be the same or different, and options of each R″ do not influence each other; and a formula Q-2 represents that each benzene ring of biphenyl has q substituents R″, the number q of the substituents R″ on the two benzene rings can be the same or different, each R″ can be the same or different, and options of each R″ do not influence each other.

In the present disclosure, the term such as “substituted or unsubstituted” means that a functional group described behind the term may have or may not have a substituent (in the below, the substituent is collectively referred to as Rc in order to facilitate description). For example, “substituted or unsubstituted aryl” refers to aryl with the substituent Rc or unsubstituted aryl. The above substituent, i.e., Rc, may be, for example, a deuterium, a cyano, a halogen group, an alkyl, a cycloalkyl, an aryl, a deuteroaryl, a haloaryl, or the like. The number of substituents may be one or more.

In the present disclosure, “a plurality of” refers to two or more, e.g., two, three, four, five, six, etc.

In the present disclosure, the number of carbon atoms in a substituted or unsubstituted functional group refers to the number of all carbon atoms. For example, if L is substituted arylene with 12 carbon atoms, the number of all carbon atoms of the arylene and substituents on the arylene is 12.

In the present disclosure, aryl refers to an optional functional group or substituent derived from an aromatic carbocyclic ring. The aryl may be monocyclic aryl (e.g., phenyl) or polycyclic aryl, in other words, the aryl can be monocyclic aryl, fused aryl, two or more monocyclic aryl conjugatedly connected through carbon-carbon bonds, monocyclic aryl and fused aryl which are conjugatedly connected through a carbon-carbon bond, and two or more fused aryl conjugatedly connected through carbon-carbon bonds. That is, unless otherwise specified, two or more aromatic groups conjugatedly connected through carbon-carbon bonds may also be regarded as the aryl of the present disclosure. The fused aryl may, for example, include bicyclic fused aryl (e.g., naphthyl), tricyclic fused aryl (e.g., phenanthryl, fluorenyl, and anthryl), and the like. The aryl does not contain heteroatoms such as B, N, O, S, P, Se and Si. Examples of the aryl can include, but are not limited to, phenyl, naphthyl, fluorenyl, anthryl, phenanthryl, biphenyl, terphenyl, triphenylenediyl, perylenyl, benzo[9,10] phenanthryl, pyrenyl, benzofluoranthenyl, chrysenyl, spirobifluorenyl, and the like. In the present disclosure, the arylene involved is a divalent group formed by further loss of one hydrogen atom from aryl.

In the present disclosure, terphenyl includes

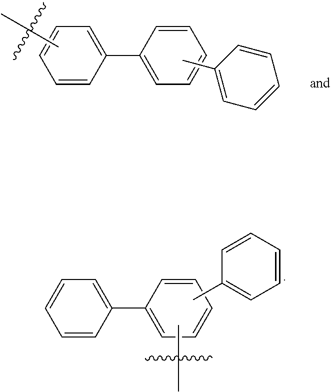

In the present disclosure, the number of carbon atoms of the substituted aryl refers to the total number of carbon atoms of the aryl and substituents on the aryl, for example, substituted aryl with 18 carbon atoms means that the total number of carbon atoms of the aryl and substituents is 18.

In the present disclosure, the number of carbon atoms of the substituted or unsubstituted aryl may be 6, 10, 12, 13, 14, 15, 16, 17, 18, 20, 24, 25 or 30. In some embodiments, the substituted or unsubstituted aryl is substituted or unsubstituted aryl with 6 to 30 carbon atoms, in other embodiments, the substituted or unsubstituted aryl is substituted or unsubstituted aryl with 6 to 25 carbon atoms, in other embodiments, the substituted or unsubstituted aryl is substituted or unsubstituted aryl with 6 to 20 carbon atoms, and in other embodiments, the substituted or unsubstituted aryl is substituted or unsubstituted aryl with 6 to 12 carbon atoms.

In the present disclosure, aryl as a substituent of L and Ar is, for example, but not limited to, phenyl, naphthyl, and the like.

In the present disclosure, the alkyl with 1 to 10 carbon atoms may include linear alkyl with 1 to 10 carbon atoms and branched alkyl with 3 to 10 carbon atoms. The number of carbon atoms of the alkyl may be, for example, 1, 2, 3, 4, 5, 6, 7, 8, 9, or 10, and specific examples of the alkyl include, but are not limited to, methyl, ethyl, n-propyl, isopropyl, n-butyl, isobutyl, tert-butyl, n-pentyl, isopentyl, neopentyl, n-hexyl, and the like.

In the present disclosure, the halogen group may be, for example, fluorine, chlorine, bromine or iodine.

In the present disclosure, specific examples of haloalkyl include, but are not limited to, trifluoromethyl.

In the present disclosure, specific examples of deuteroalkyl include, but are not limited to, trideuteromethyl.

In the present disclosure, deuteroaryl refers to aryl containing at least one a deuterium substituent, and specific examples of the deuteroaryl include, but are not limited to, pentadeuterophenyl and pentadeuterobiphenyl.

In the present disclosure, the number of carbon atoms of cycloalkyl with 3 to 10 carbon atoms may be, for example, 3, 4, 5, 6, 7, 8, or 10. Specific examples of the cycloalkyl include, but are not limited to, cyclopentyl, cyclohexyl, and adamantyl.

In the present disclosure, an unpositioned connecting bond refers to a single bond “-ξ-” extending from a ring system, which means that one end of the connecting bond can be connected with any position in the ring system through which the bond penetrates, and the other end of the connecting bond is connected with the remaining part of a compound molecule. For example, as shown in the following formula (f), naphthyl represented by the formula (f) is connected to other positions of a molecule through two unpositioned connecting bonds penetrating a dicyclic ring, and its meaning includes any one possible connecting mode represented by formulae (f-1) to (f-10).

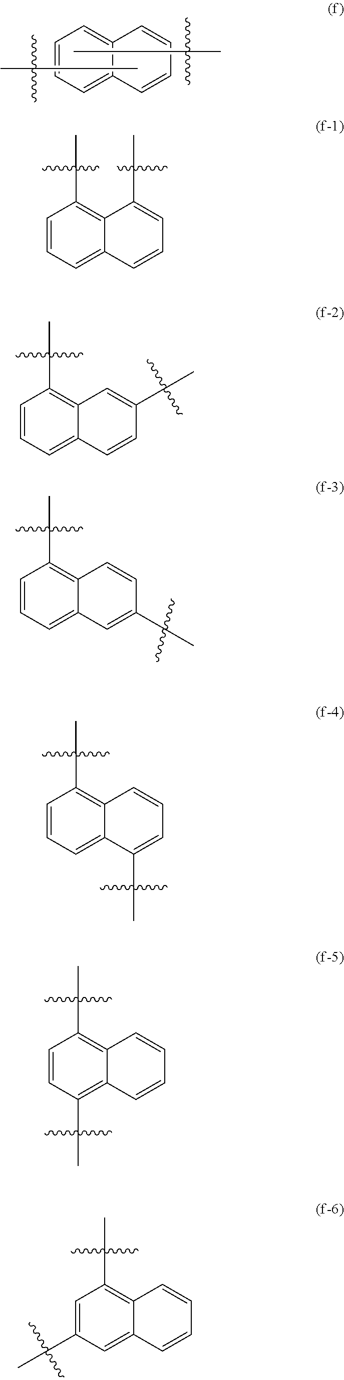

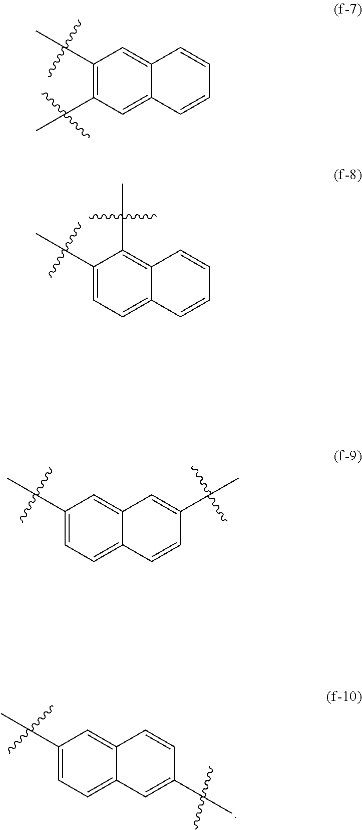 [SMILES: *c1cc2ccccc2cc1* ; *c1ccc2ccccc2c1* ; *c1ccc2ccc(*)cc2c1 ; *c1ccc2cc(*)ccc2c1]

For another example, as shown in the following formula (X′), dibenzofuranyl represented by the formula (X′) is connected with other positions of a molecule through one unpositioned connecting bond extending from the middle of a benzene ring on one side, and its meaning includes any one possible connecting mode represented by formulae (X′-1) to (X′-4).

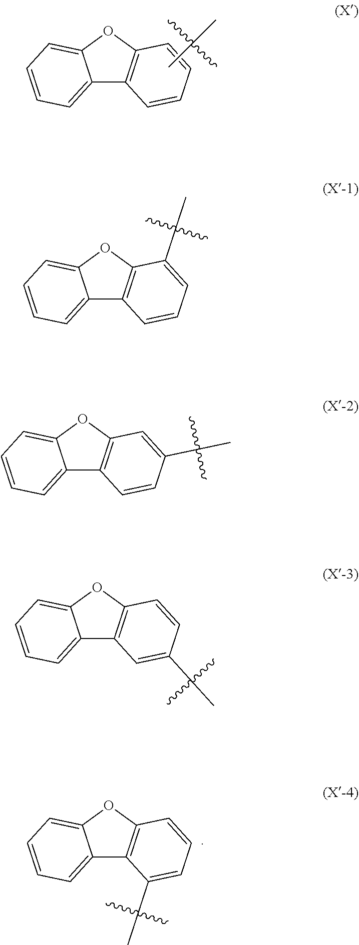

In some embodiments of the present disclosure, the nitrogen-containing compound is selected from compounds represented by a Formula C or a Formula B:

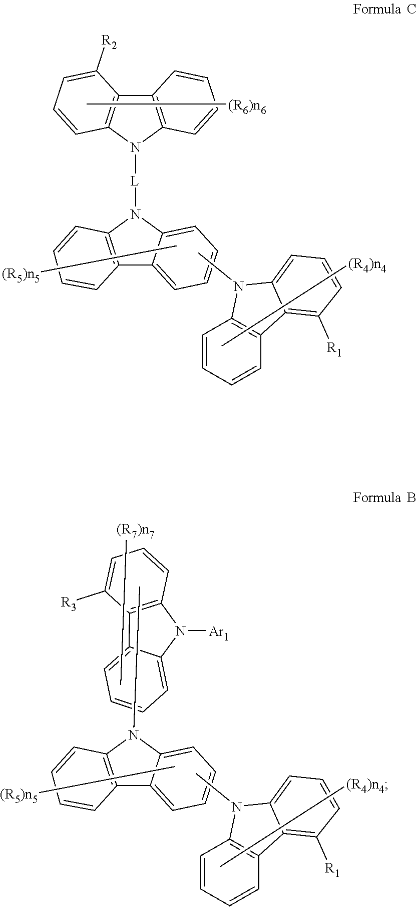

in the Formula C, R₁ and R₂ are the same or different, and are respectively and independently selected from a hydrogen, a deuterium, an aryl with 6 to 12 carbon atoms, or a deuteroaryl with 6 to 12 carbon atoms, and at least one of R₁ and R₂ is an aryl with 6 to 12 carbon atoms or a deuteroaryl with 6 to 12 carbon atoms;
each R₄, each Rs and each R₆ are the same or different, and are respectively and independently selected from a deuterium, an aryl with 6 to 12 carbon atoms or a deuteroaryl with 6 to 12 carbon atoms;
n₄ is the number of R₄, and is selected from 0, 1, 2, 3, 4, 5, 6 or 7, and when n₄ is greater than 1, each R₄ is the same or different;
n₅ is the number of R₅, and is selected from 0, 1, 2, 3, 4, 5, 6 or 7, and when n₅ is greater than 1, each R₅ is the same or different;
no is the number of R₆, and is selected from 0, 1, 2, 3, 4, 5, 6 or 7, and when no is greater than 1, each R₆ is the same or different;
L is selected from a substituted or unsubstituted arylene with 6 to 12 carbon atoms;
substituent(s) of L are the same or different, and are respectively and independently selected from a deuterium, a phenyl or a pentadeuterophenyl.

In the Formula B, R₁ and R₃ are the same or different, and are respectively and independently selected from a hydrogen, a deuterium, an aryl with 6 to 12 carbon atoms, or a deuteroaryl with 6 to 12 carbon atoms, and at least one of R₁ and R₃ is an aryl with 6 to 12 carbon atoms or a deuteroaryl with 6 to 12 carbon atoms;
each R₄, each Rs and each R₇ are the same or different, and are respectively and independently selected from a deuterium, an aryl with 6 to 12 carbon atoms or a deuteroaryl with 6 to 12 carbon atoms;
n₄ is the number of R₄, and is selected from 0, 1, 2, 3, 4, 5, 6 or 7, and each R₄ is the same or different;
n₅ is the number of R₅, and is selected from 0, 1, 2, 3, 4, 5, 6 or 7, and each R₅ is the same or different;
n₇ is the number of R₇, and is selected from 0, 1, 2, 3, 4, 5 or 6, and each R₇ is the same or different;
Ar₁ is selected from a substituted or unsubstituted aryl with 6 to 30 carbon atoms; and
substituent(s) of Ar₁ are the same or different, and are respectively and independently selected from a deuterium, a halogen group, a cyano, an alkyl with 1 to 10 carbon atoms, a cycloalkyl with 3 to 10 carbon atoms, an aryl with 6 to 12 carbon atoms, a deuteroaryl with 6 to 12 carbon atoms, or a haloaryl with 6 to 12 carbon atoms.

In some specific embodiments of the present disclosure, the nitrogen-containing compound is selected from compounds represented by a Formula AA, a Formula AB, a Formula AC, a Formula BA, a Formula BB, a Formula BC, or a Formula BE:

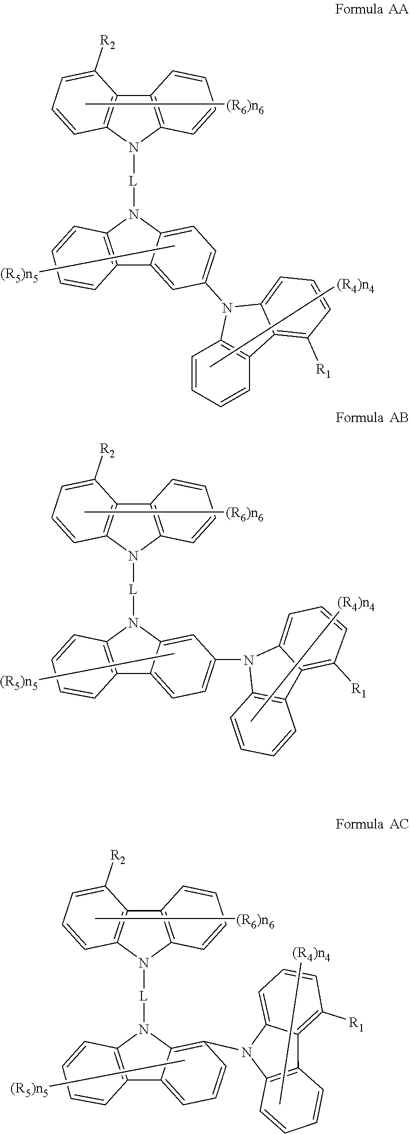

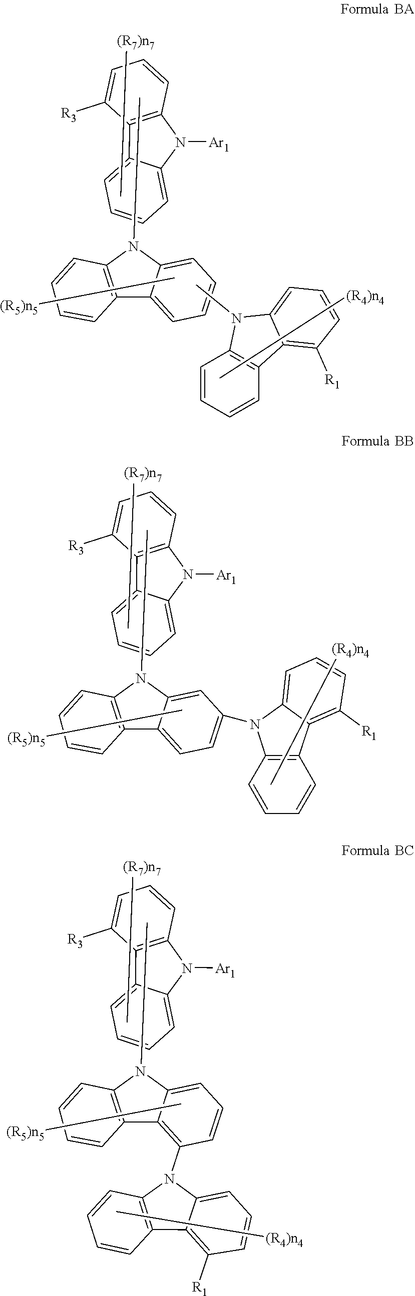

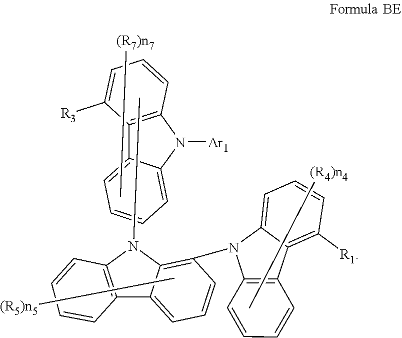

In some embodiments of the present disclosure, L is selected from a substituted or unsubstituted phenylene or a substituted or unsubstituted biphenylene.

Optionally, substituent(s) of L are the same or different, and are respectively and independently selected from a deuterium, a phenyl or a pentadeuterophenyl.

In other embodiments of the present disclosure, L is selected from the group consisting of:

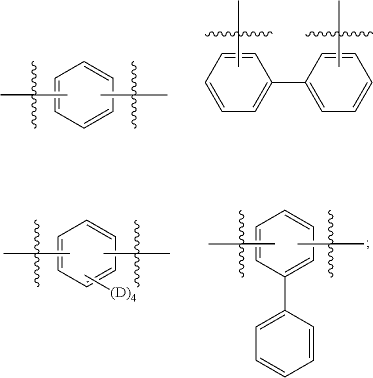

In some specific embodiments of the present disclosure, L is selected from the group consisting of:

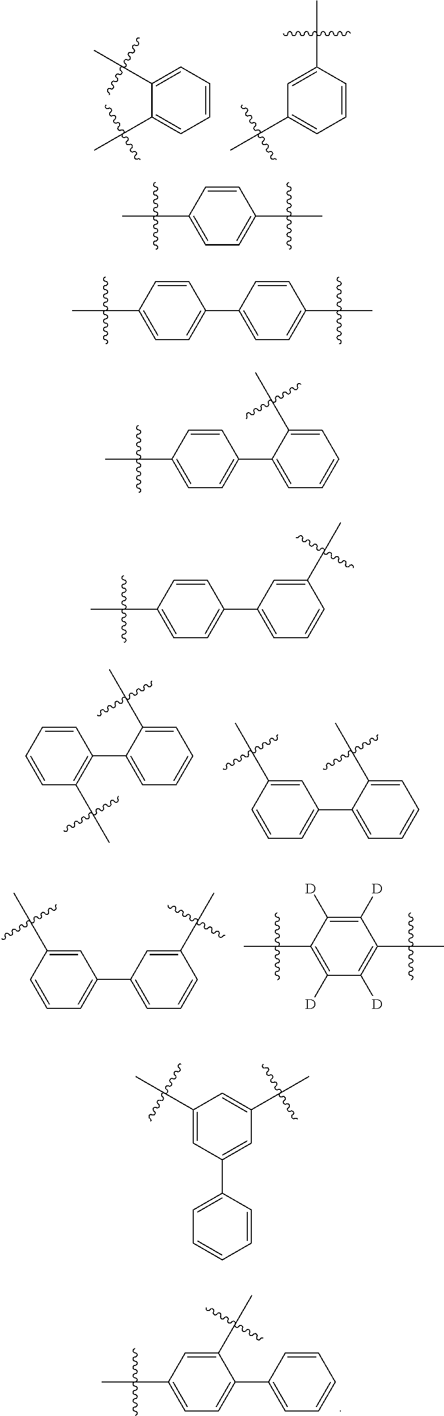

In some embodiments of the present disclosure, Ar₁ is selected from a substituted or unsubstituted aryl with 6 to 20 carbon atoms.

Optionally, substituent(s) of Ar₁ are the same or different, and are respectively and independently selected from a deuterium, a fluorine, a cyano, an alkyl with 1 to 5 carbon atoms, a phenyl or a pentadeuterophenyl.

Further optionally, Ar₁ is selected from a substituted or unsubstituted aryl with 6 to 12 carbon atoms.

In some embodiments of the present disclosure, Ar₁ is selected from a substituted or unsubstituted phenyl, a substituted or unsubstituted biphenyl, a substituted or unsubstituted phenanthryl, a substituted or unsubstituted naphthyl, a substituted or unsubstituted terphenyl, or a substituted or unsubstituted fluorenyl.

Optionally, substituent(s) of Ar₁ are the same or different, and are respectively and independently selected from a deuterium, a fluorine, a cyano, a methyl, an ethyl, a n-propyl, an isopropyl, a tert-butyl, a phenyl or a pentadeuterophenyl.

In other embodiments of the present disclosure, Ar₁ is selected from the group consisting of:

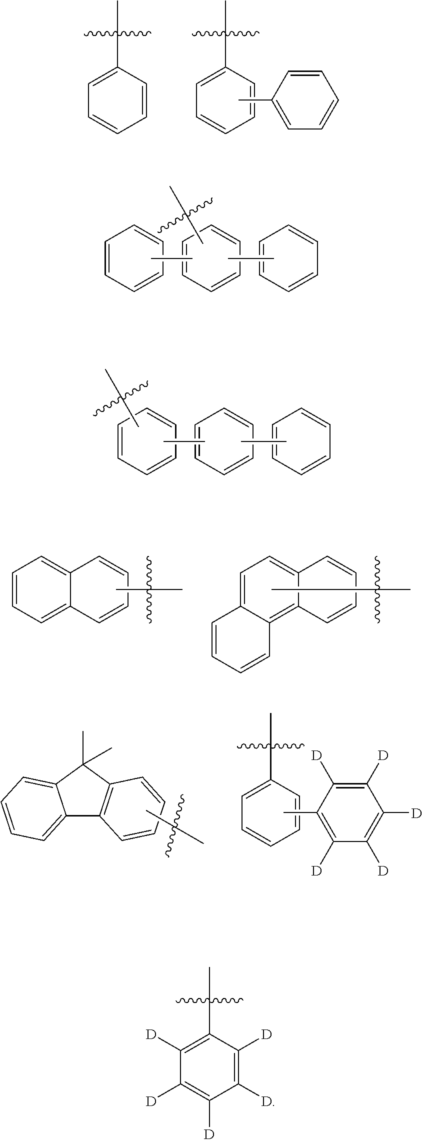

In some specific embodiments of the present disclosure, Ar₁ is selected from the group consisting of:

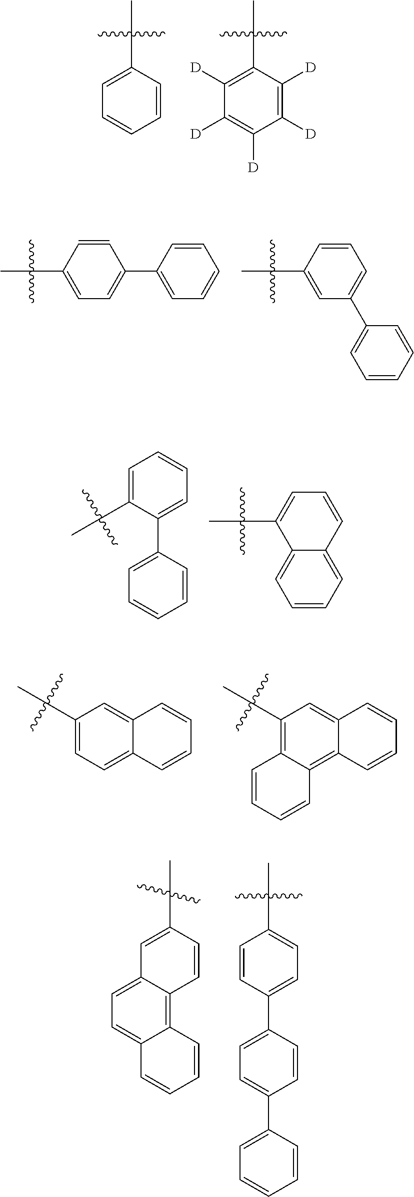

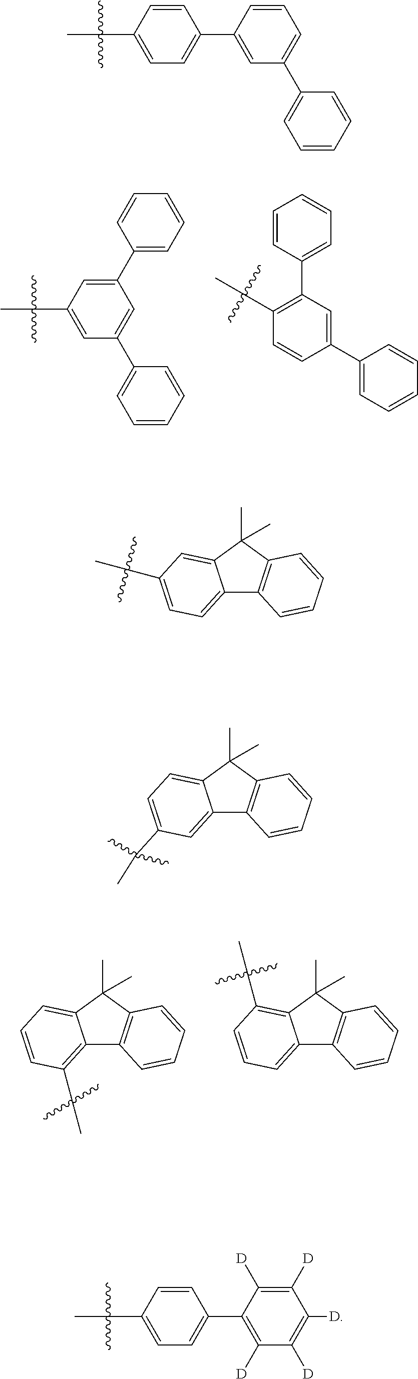

In some embodiments of the present disclosure, each R₄, each R₅, each R₆ and each R₇ is selected from a deuterium.

In some embodiments of the present disclosure, R₁ and R₂ are the same or different, and are respectively and independently selected from a hydrogen, a deuterium, a phenyl or a pentadeuterophenyl, and at least one of R₁ and R₂ is a phenyl or a pentadeuterophenyl.

Optionally, R₁ is a hydrogen or a deuterium and R₂ is a phenyl.

Optionally, R₁ is a hydrogen or a deuterium and R₂ is a pentadeuterophenyl.

Optionally, R₂ is a hydrogen or a deuterium and R₁ is a phenyl.

Optionally, R₂ is a hydrogen or a deuterium and R₁ is a pentadeuterophenyl.

Optionally, R₁ is a phenyl and R₂ is a pentadeuterophenyl.

Optionally, R₂ is a phenyl and R₁ is a pentadeuterophenyl.

Optionally, R₁ and R₂ are both phenyl.

Optionally, R₁ and R₂ are both pentadeuterophenyl.

In other embodiments of the present disclosure, R₁ and R₃ are the same or different, and are respectively and independently selected from a hydrogen, a deuterium, a phenyl or a pentadeuterophenyl, and at least one of R₁ and R₃ is a phenyl or a pentadeuterophenyl.

Optionally, R₁ is a hydrogen or a deuterium and R₃ is a phenyl.

Optionally, R₁ is a hydrogen or a deuterium and R₃ is a pentadeuterophenyl.

Optionally, R₃ is a hydrogen or a deuterium and R₁ is a phenyl.

Optionally, R₃ is a hydrogen or a deuterium and R₁ is a pentadeuterophenyl.

Optionally, R₁ is a phenyl and R₃ is a pentadeuterophenyl.

Optionally, R₃ is a phenyl and R₁ is a pentadeuterophenyl.

Optionally, R₁ and R₃ are both phenyl.

Optionally, R₁ and R₃ are both pentadeuterophenyl.

Optionally, the nitrogen-containing compound is selected from the group consisting of the following compounds:

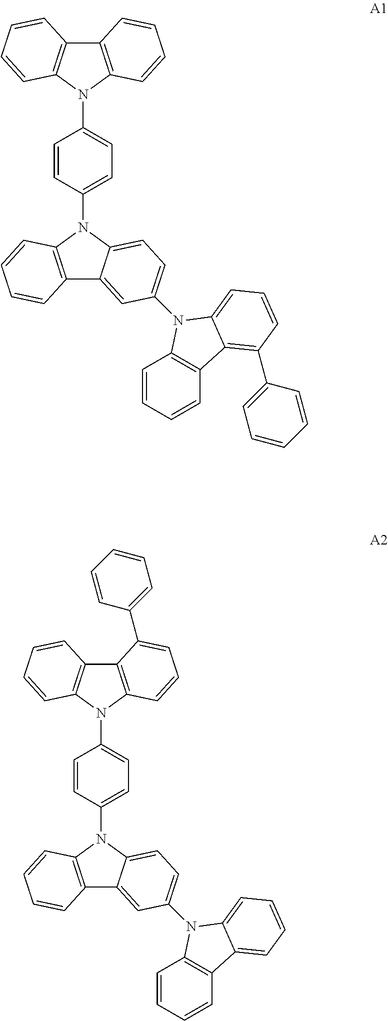 [SMILES: c1ccc(-c2cccc3c2c2ccccc2n3-c2ccc3c(c2)c2ccccc2n3-c2ccc(-n3c4ccccc4c4ccccc43)cc2)cc1 ; c1ccc(-c2cccc3c2c2ccccc2n3-c2ccc(-n3c4ccccc4c4cc(-n5c6ccccc6c6ccccc65)ccc43)cc2)cc1]

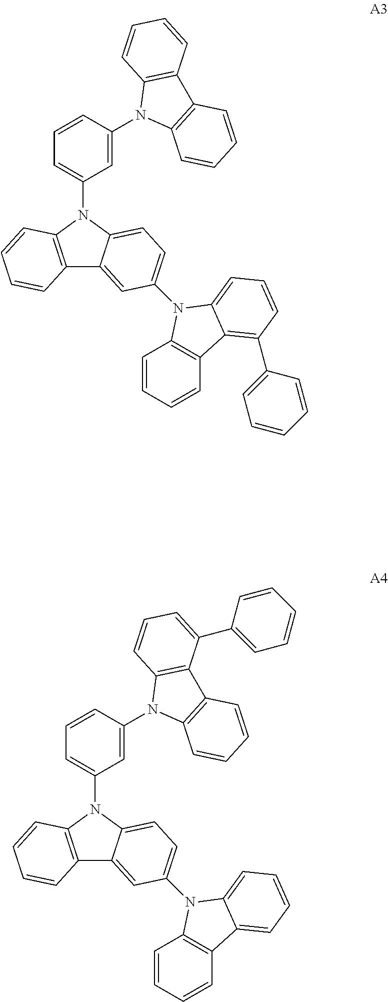 [SMILES: c1ccc(-c2cccc3c2c2ccccc2n3-c2ccc3c(c2)c2ccccc2n3-c2cccc(-n3c4ccccc4c4ccccc43)c2)cc1 ; c1ccc(-c2cccc3c2c2ccccc2n3-c2cccc(-n3c4ccccc4c4cc(-n5c6ccccc6c6ccccc65)ccc43)c2)cc1]

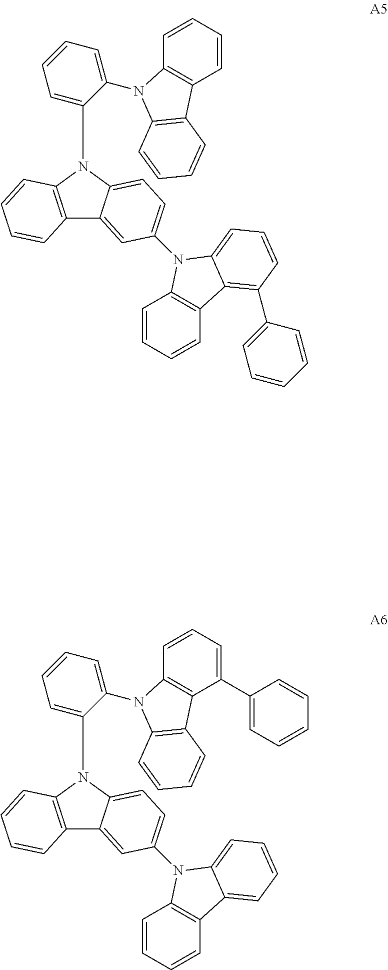 [SMILES: c1ccc(-c2cccc3c2c2ccccc2n3-c2ccc3c(c2)c2ccccc2n3-c2ccccc2-n2c3ccccc3c3ccccc32)cc1 ; c1ccc(-c2cccc3c2c2ccccc2n3-c2ccccc2-n2c3ccccc3c3cc(-n4c5ccccc5c5ccccc54)ccc32)cc1]

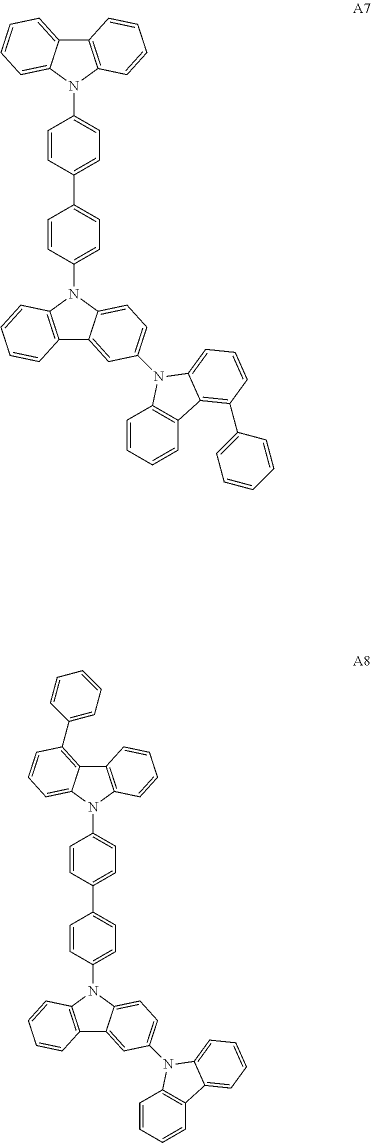 [SMILES: c1ccc(-c2cccc3c2c2ccccc2n3-c2ccc3c(c2)c2ccccc2n3-c2ccc(-c3ccc(-n4c5ccccc5c5ccccc54)cc3)cc2)cc1 ; c1ccc(-c2cccc3c2c2ccccc2n3-c2ccc(-c3ccc(-n4c5ccccc5c5cc(-n6c7ccccc7c7ccccc76)ccc54)cc3)cc2)cc1]

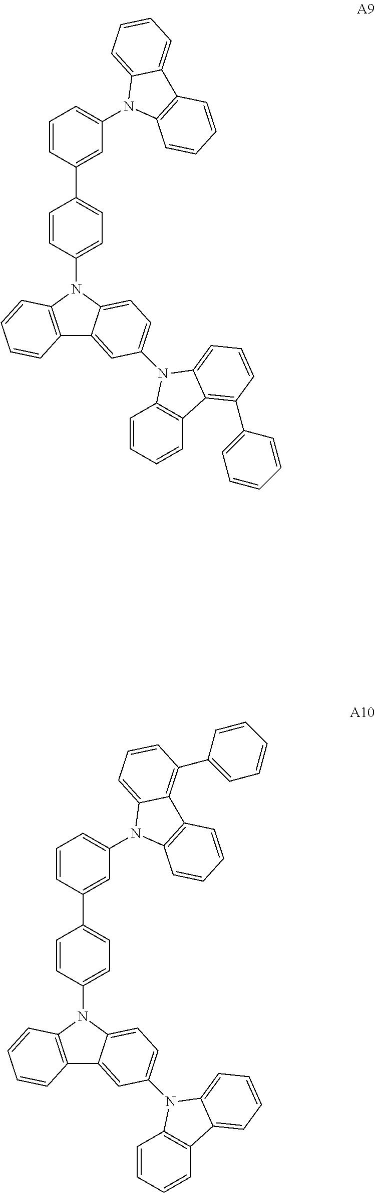 [SMILES: c1ccc(-c2cccc3c2c2ccccc2n3-c2ccc3c(c2)c2ccccc2n3-c2ccc(-c3cccc(-n4c5ccccc5c5ccccc54)c3)cc2)cc1 ; c1ccc(-c2cccc3c2c2ccccc2n3-c2cccc(-c3ccc(-n4c5ccccc5c5cc(-n6c7ccccc7c7ccccc76)ccc54)cc3)c2)cc1]

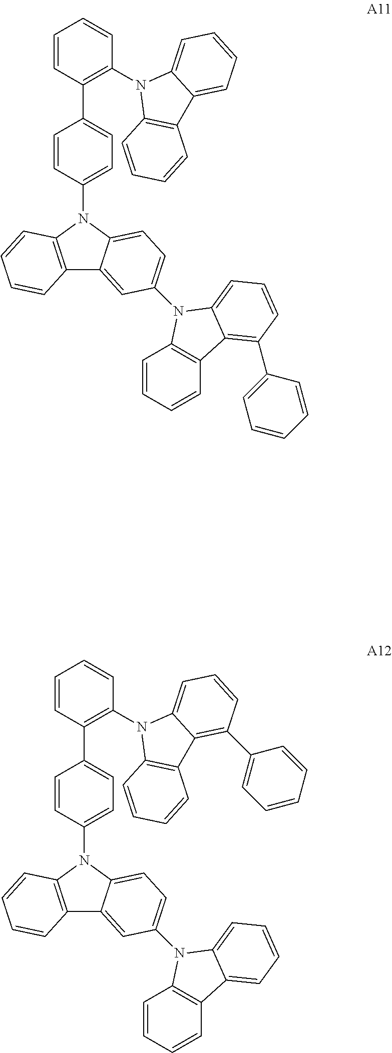 [SMILES: c1ccc(-c2cccc3c2c2ccccc2n3-c2ccc3c(c2)c2ccccc2n3-c2ccc(-c3ccccc3-n3c4ccccc4c4ccccc43)cc2)cc1 ; c1ccc(-c2cccc3c2c2ccccc2n3-c2ccccc2-c2ccc(-n3c4ccccc4c4cc(-n5c6ccccc6c6ccccc65)ccc43)cc2)cc1]

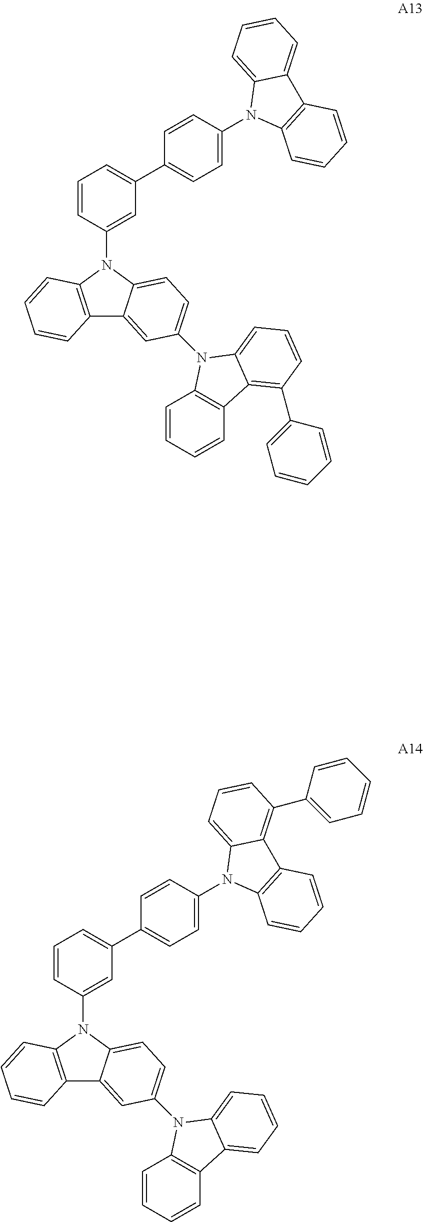 [SMILES: c1ccc(-c2cccc3c2c2ccccc2n3-c2ccc3c(c2)c2ccccc2n3-c2cccc(-c3ccc(-n4c5ccccc5c5ccccc54)cc3)c2)cc1 ; c1ccc(-c2cccc3c2c2ccccc2n3-c2ccc(-c3cccc(-n4c5ccccc5c5cc(-n6c7ccccc7c7ccccc76)ccc54)c3)cc2)cc1]

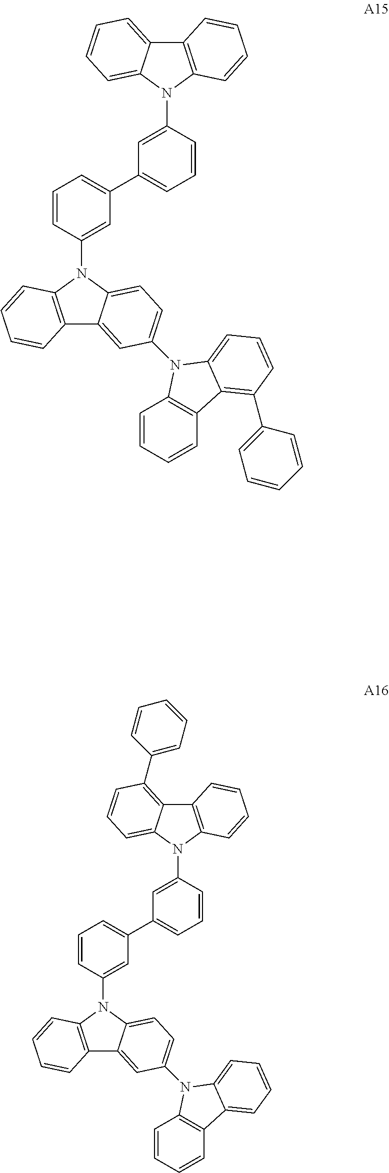 [SMILES: c1ccc(-c2cccc3c2c2ccccc2n3-c2ccc3c(c2)c2ccccc2n3-c2cccc(-c3cccc(-n4c5ccccc5c5ccccc54)c3)c2)cc1 ; c1ccc(-c2cccc3c2c2ccccc2n3-c2cccc(-c3cccc(-n4c5ccccc5c5cc(-n6c7ccccc7c7ccccc76)ccc54)c3)c2)cc1]

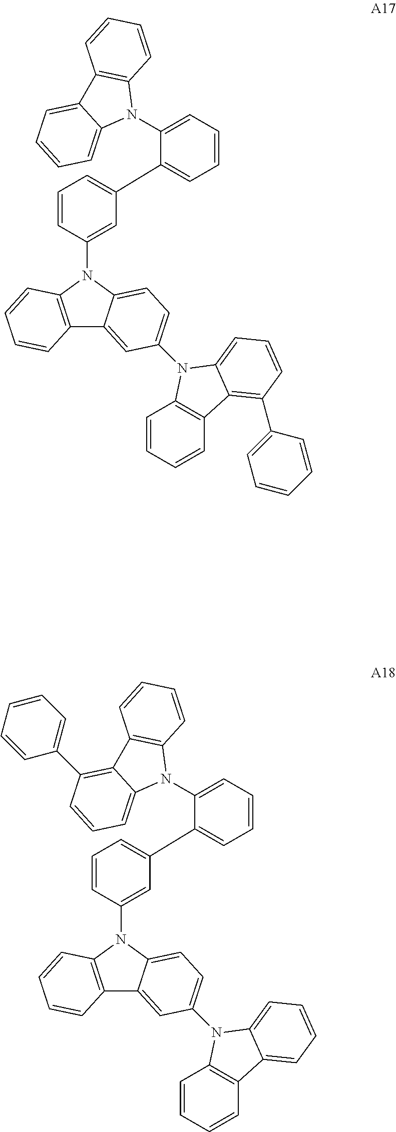 [SMILES: c1ccc(-c2cccc3c2c2ccccc2n3-c2ccc3c(c2)c2ccccc2n3-c2cccc(-c3ccccc3-n3c4ccccc4c4ccccc43)c2)cc1 ; c1ccc(-c2cccc3c2c2ccccc2n3-c2ccccc2-c2cccc(-n3c4ccccc4c4cc(-n5c6ccccc6c6ccccc65)ccc43)c2)cc1]

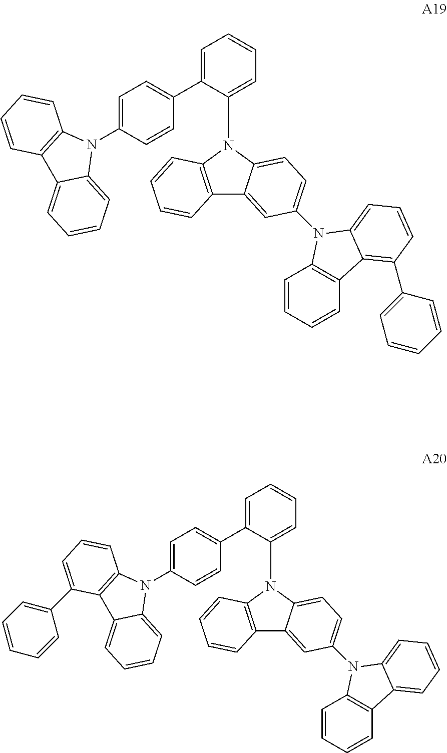 [SMILES: c1ccc(-c2cccc3c2c2ccccc2n3-c2ccc3c(c2)c2ccccc2n3-c2ccccc2-c2ccc(-n3c4ccccc4c4ccccc43)cc2)cc1 ; c1ccc(-c2cccc3c2c2ccccc2n3-c2ccc(-c3ccccc3-n3c4ccccc4c4cc(-n5c6ccccc6c6ccccc65)ccc43)cc2)cc1]

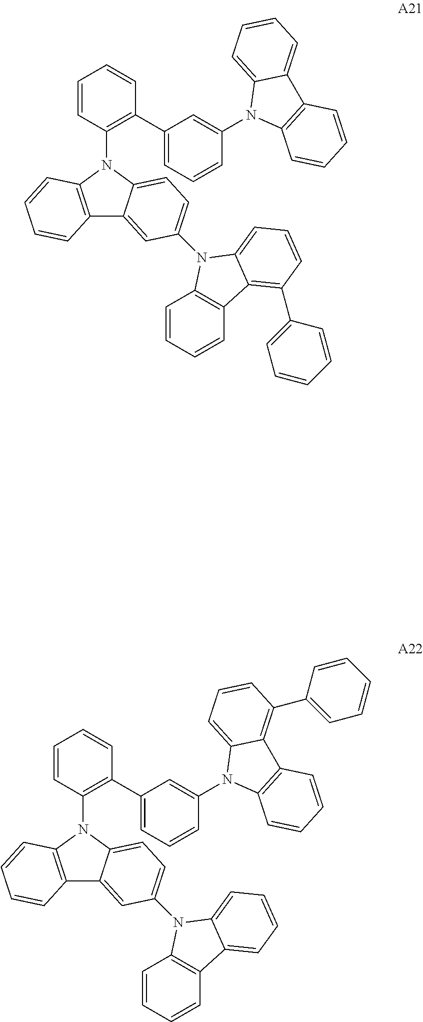 [SMILES: c1ccc(-c2cccc3c2c2ccccc2n3-c2ccc3c(c2)c2ccccc2n3-c2ccccc2-c2cccc(-n3c4ccccc4c4ccccc43)c2)cc1 ; c1ccc(-c2cccc3c2c2ccccc2n3-c2cccc(-c3ccccc3-n3c4ccccc4c4cc(-n5c6ccccc6c6ccccc65)ccc43)c2)cc1]

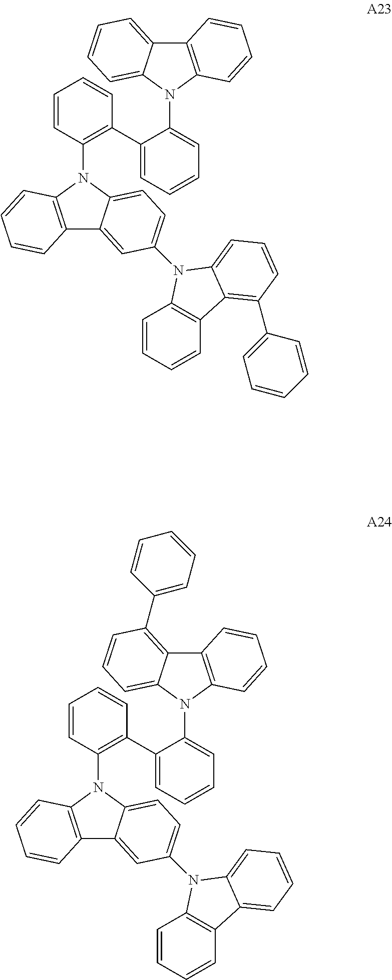 [SMILES: c1ccc(-c2cccc3c2c2ccccc2n3-c2ccc3c(c2)c2ccccc2n3-c2ccccc2-c2ccccc2-n2c3ccccc3c3ccccc32)cc1 ; c1ccc(-c2cccc3c2c2ccccc2n3-c2ccccc2-c2ccccc2-n2c3ccccc3c3cc(-n4c5ccccc5c5ccccc54)ccc32)cc1]

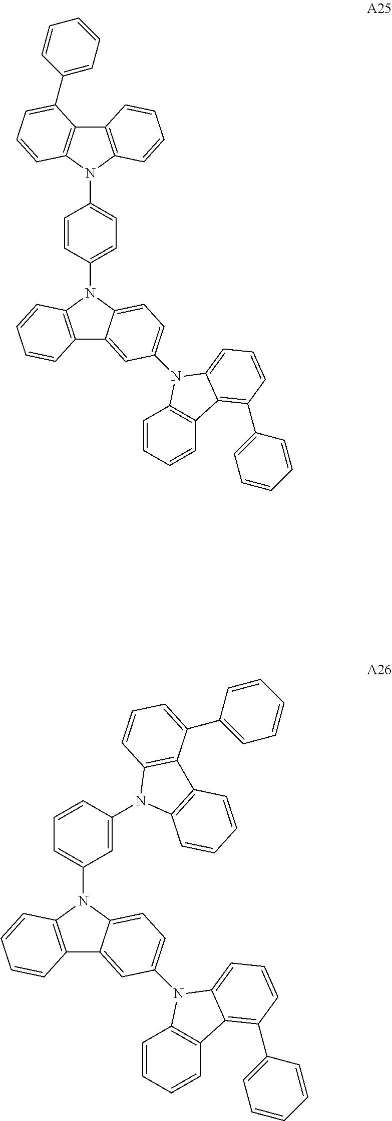 [SMILES: c1ccc(-c2cccc3c2c2ccccc2n3-c2ccc(-n3c4ccccc4c4cc(-n5c6ccccc6c6c(-c7ccccc7)cccc65)ccc43)cc2)cc1 ; c1ccc(-c2cccc3c2c2ccccc2n3-c2cccc(-n3c4ccccc4c4cc(-n5c6ccccc6c6c(-c7ccccc7)cccc65)ccc43)c2)cc1]

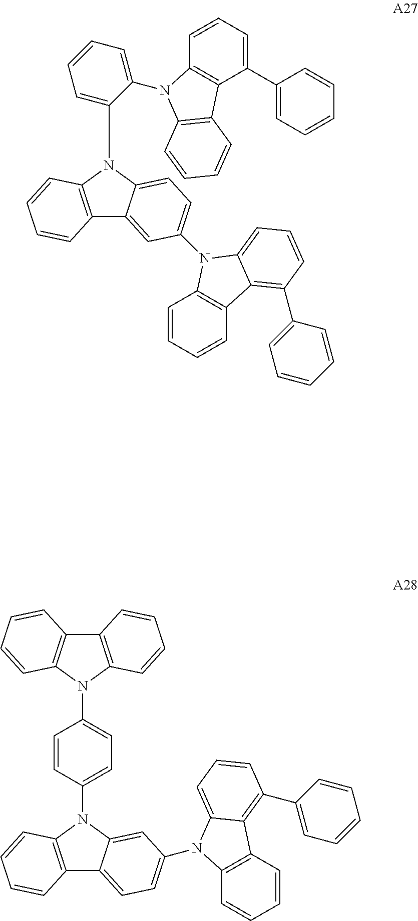 [SMILES: c1ccc(-c2cccc3c2c2ccccc2n3-c2ccc3c(c2)c2ccccc2n3-c2ccccc2-n2c3ccccc3c3c(-c4ccccc4)cccc32)cc1 ; c1ccc(-c2cccc3c2c2ccccc2n3-c2ccc3c4ccccc4n(-c4ccc(-n5c6ccccc6c6ccccc65)cc4)c3c2)cc1]

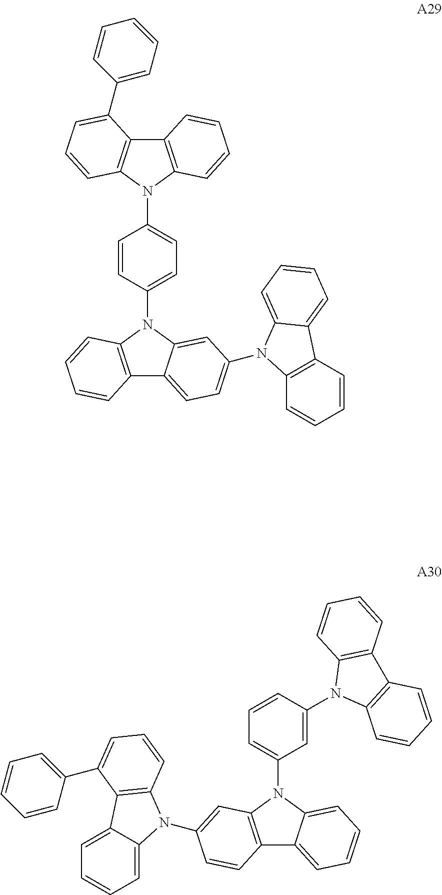 [SMILES: c1ccc(-c2cccc3c2c2ccccc2n3-c2ccc(-n3c4ccccc4c4ccc(-n5c6ccccc6c6ccccc65)cc43)cc2)cc1 ; c1ccc(-c2cccc3c2c2ccccc2n3-c2ccc3c4ccccc4n(-c4cccc(-n5c6ccccc6c6ccccc65)c4)c3c2)cc1]

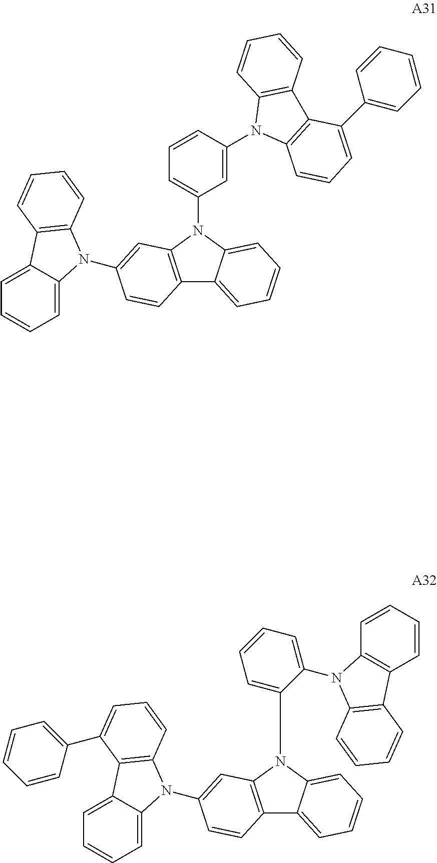 [SMILES: c1ccc(-c2cccc3c2c2ccccc2n3-c2cccc(-n3c4ccccc4c4ccc(-n5c6ccccc6c6ccccc65)cc43)c2)cc1 ; c1ccc(-c2cccc3c2c2ccccc2n3-c2ccc3c4ccccc4n(-c4ccccc4-n4c5ccccc5c5ccccc54)c3c2)cc1]

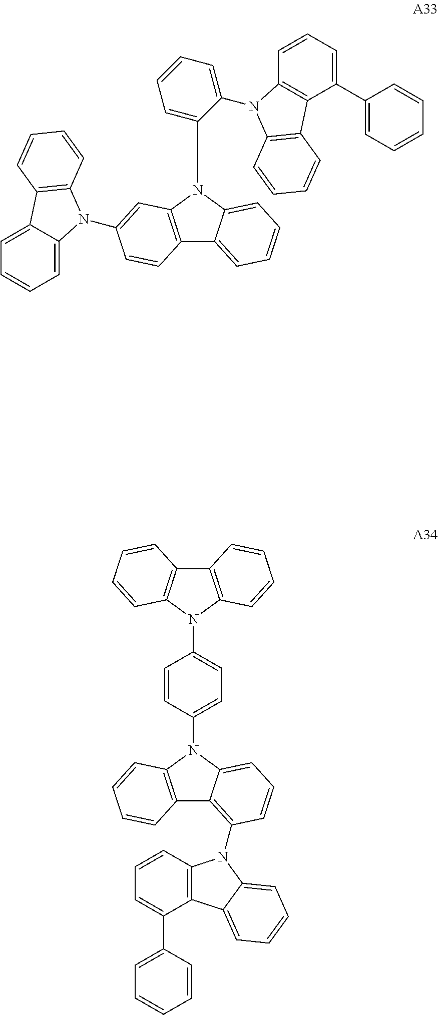 [SMILES: c1ccc(-c2cccc3c2c2ccccc2n3-c2ccccc2-n2c3ccccc3c3ccc(-n4c5ccccc5c5ccccc54)cc32)cc1 ; c1ccc(-c2cccc3c2c2ccccc2n3-c2cccc3c2c2ccccc2n3-c2ccc(-n3c4ccccc4c4ccccc43)cc2)cc1]

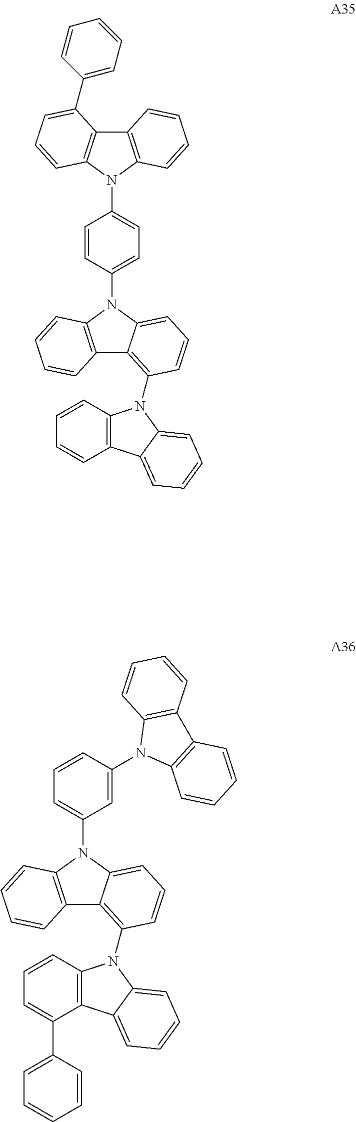 [SMILES: c1ccc(-c2cccc3c2c2ccccc2n3-c2ccc(-n3c4ccccc4c4c(-n5c6ccccc6c6ccccc65)cccc43)cc2)cc1 ; c1ccc(-c2cccc3c2c2ccccc2n3-c2cccc3c2c2ccccc2n3-c2cccc(-n3c4ccccc4c4ccccc43)c2)cc1]

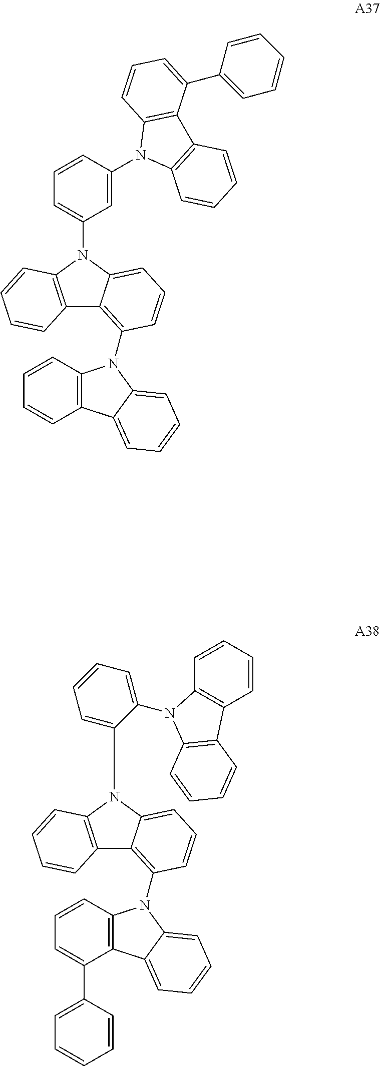 [SMILES: c1ccc(-c2cccc3c2c2ccccc2n3-c2cccc(-n3c4ccccc4c4c(-n5c6ccccc6c6ccccc65)cccc43)c2)cc1 ; c1ccc(-c2cccc3c2c2ccccc2n3-c2cccc3c2c2ccccc2n3-c2ccccc2-n2c3ccccc3c3ccccc32)cc1]

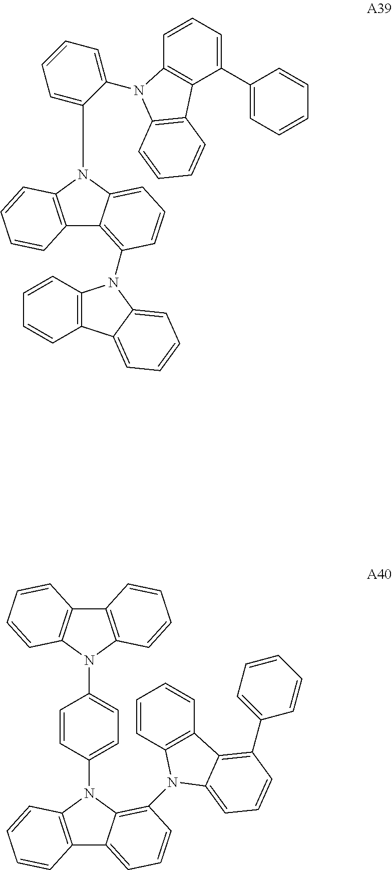 [SMILES: c1ccc(-c2cccc3c2c2ccccc2n3-c2ccccc2-n2c3ccccc3c3c(-n4c5ccccc5c5ccccc54)cccc32)cc1 ; c1ccc(-c2cccc3c2c2ccccc2n3-c2cccc3c4ccccc4n(-c4ccc(-n5c6ccccc6c6ccccc65)cc4)c23)cc1]

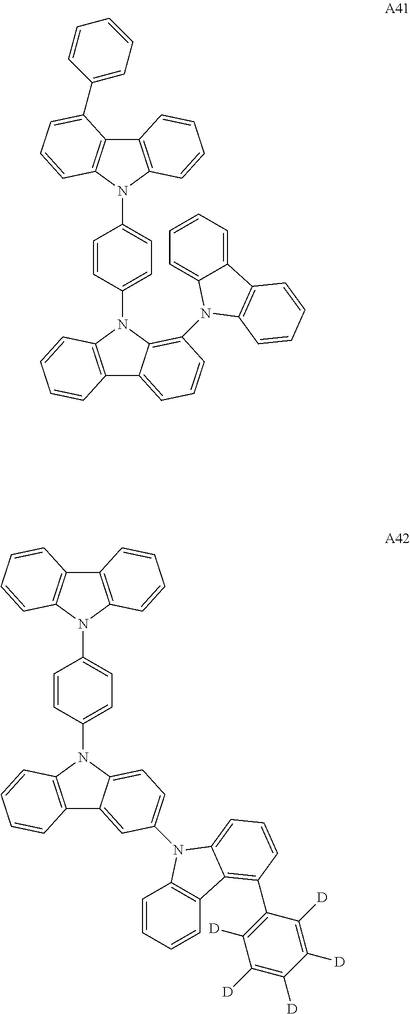 [SMILES: c1ccc(-c2cccc3c2c2ccccc2n3-c2ccc(-n3c4ccccc4c4cccc(-n5c6ccccc6c6ccccc65)c43)cc2)cc1 ; [2H]c1c([2H])c([2H])c(-c2cccc3c2c2ccccc2n3-c2ccc3c(c2)c2ccccc2n3-c2ccc(-n3c4ccccc4c4ccccc43)cc2)c([2H])c1[2H]]

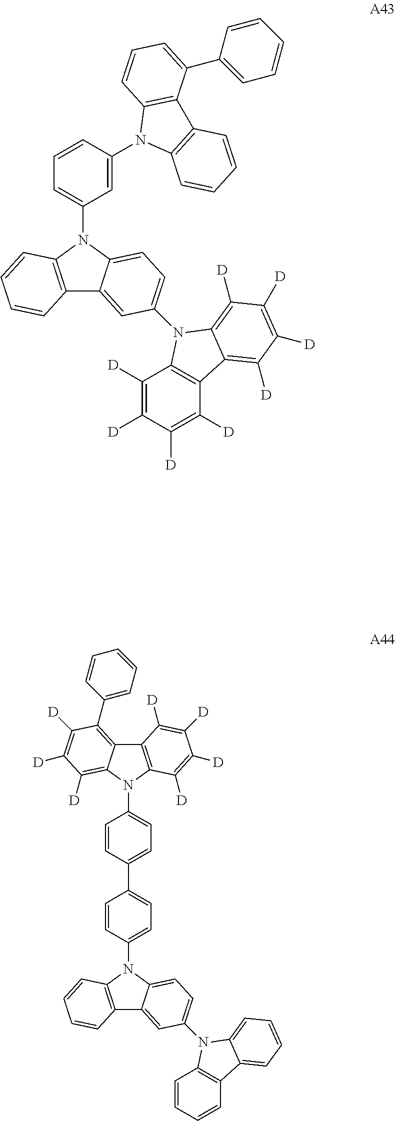 [SMILES: [2H]c1c([2H])c([2H])c2c(c1[2H])c1c([2H])c([2H])c([2H])c([2H])c1n2-c1ccc2c(c1)c1ccccc1n2-c1cccc(-n2c3ccccc3c3c(-c4ccccc4)cccc32)c1 ; [2H]c1c([2H])c([2H])c2c(c1[2H])c1c(-c3ccccc3)c([2H])c([2H])c([2H])c1n2-c1ccc(-c2ccc(-n3c4ccccc4c4cc(-n5c6ccccc6c6ccccc65)ccc43)cc2)cc1]

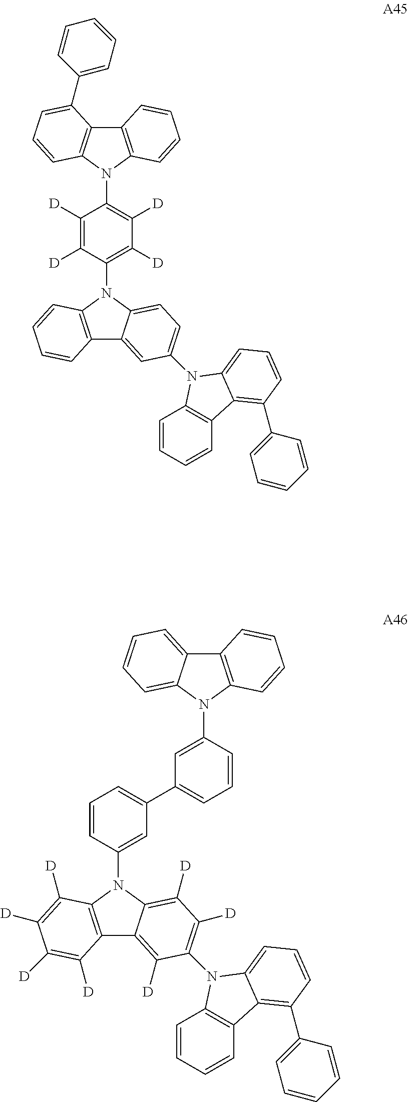 [SMILES: [2H]c1c([2H])c(-n2c3ccccc3c3c(-c4ccccc4)cccc32)c([2H])c([2H])c1-n1c2ccccc2c2cc(-n3c4ccccc4c4c(-c5ccccc5)cccc43)ccc21 ; [2H]c1c([2H])c([2H])c2c(c1[2H])c1c([2H])c(-n3c4ccccc4c4c(-c5ccccc5)cccc43)c([2H])c([2H])c1n2-c1cccc(-c2cccc(-n3c4ccccc4c4ccccc43)c2)c1]

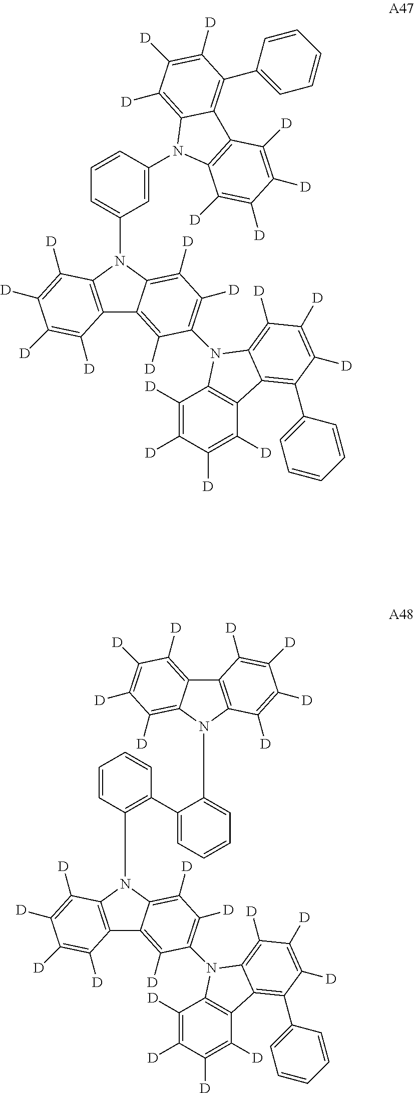 [SMILES: [2H]c1c([2H])c([2H])c2c(c1[2H])c1c([2H])c(-n3c4c([2H])c([2H])c([2H])c([2H])c4c4c(-c5ccccc5)c([2H])c([2H])c([2H])c43)c([2H])c([2H])c1n2-c1cccc(-n2c3c([2H])c([2H])c([2H])c([2H])c3c3c(-c4ccccc4)c([2H])c([2H])c([2H])c32)c1 ; [2H]c1c([2H])c([2H])c2c(c1[2H])c1c([2H])c([2H])c([2H])c([2H])c1n2-c1ccccc1-c1ccccc1-n1c2c([2H])c([2H])c([2H])c([2H])c2c2c([2H])c(-n3c4c([2H])c([2H])c([2H])c([2H])c4c4c(-c5ccccc5)c([2H])c([2H])c([2H])c43)c([2H])c([2H])c21]

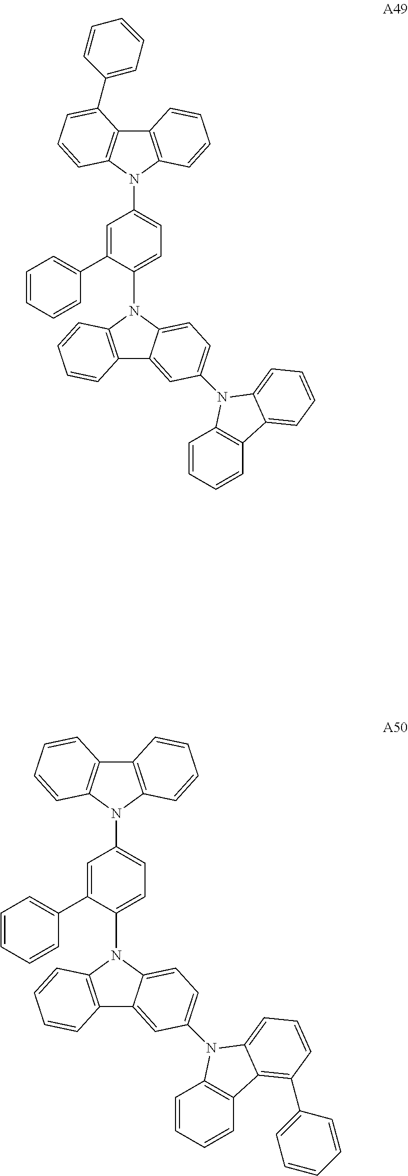 [SMILES: c1ccc(-c2cc(-n3c4ccccc4c4c(-c5ccccc5)cccc43)ccc2-n2c3ccccc3c3cc(-n4c5ccccc5c5ccccc54)ccc32)cc1 ; c1ccc(-c2cc(-n3c4ccccc4c4ccccc43)ccc2-n2c3ccccc3c3cc(-n4c5ccccc5c5c(-c6ccccc6)cccc54)ccc32)cc1]

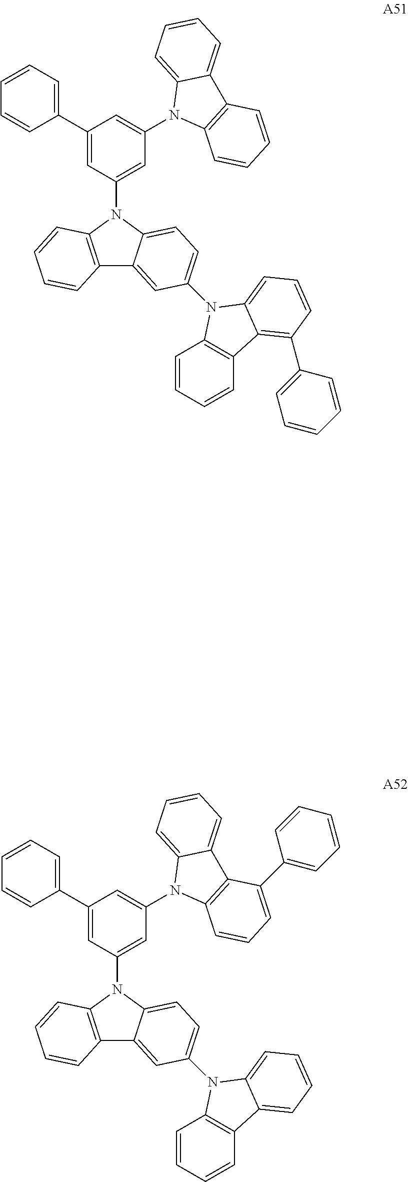 [SMILES: c1ccc(-c2cc(-n3c4ccccc4c4ccccc43)cc(-n3c4ccccc4c4cc(-n5c6ccccc6c6c(-c7ccccc7)cccc65)ccc43)c2)cc1 ; c1ccc(-c2cc(-n3c4ccccc4c4cc(-n5c6ccccc6c6ccccc65)ccc43)cc(-n3c4ccccc4c4c(-c5ccccc5)cccc43)c2)cc1]

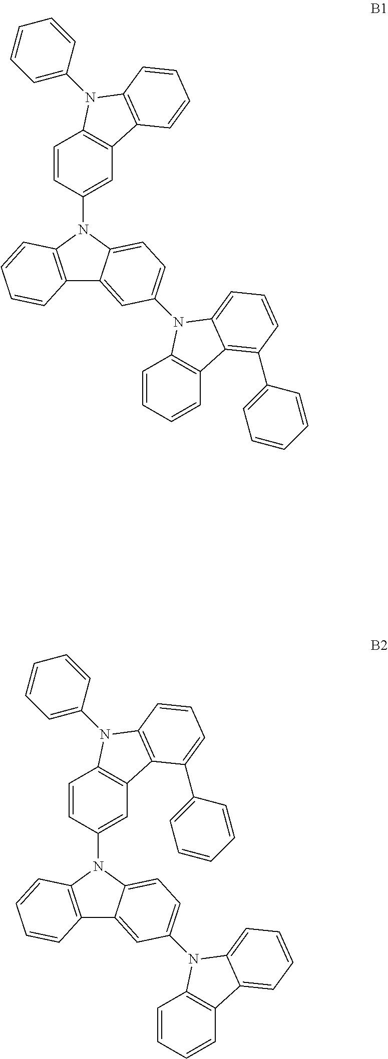 [SMILES: c1ccc(-c2cccc3c2c2ccccc2n3-c2ccc3c(c2)c2ccccc2n3-c2ccc3c(c2)c2ccccc2n3-c2ccccc2)cc1 ; c1ccc(-c2cccc3c2c2cc(-n4c5ccccc5c5cc(-n6c7ccccc7c7ccccc76)ccc54)ccc2n3-c2ccccc2)cc1]

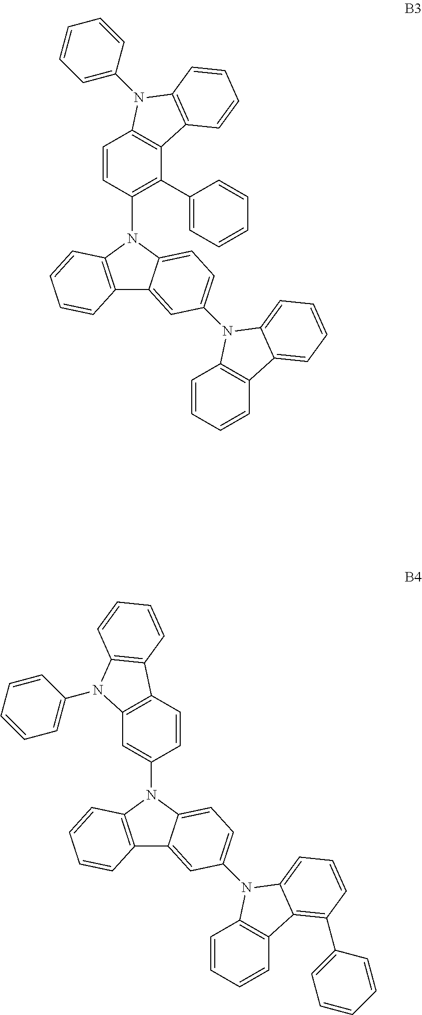 [SMILES: c1ccc(-c2c(-n3c4ccccc4c4cc(-n5c6ccccc6c6ccccc65)ccc43)ccc3c2c2ccccc2n3-c2ccccc2)cc1 ; c1ccc(-c2cccc3c2c2ccccc2n3-c2ccc3c(c2)c2ccccc2n3-c2ccc3c4ccccc4n(-c4ccccc4)c3c2)cc1]

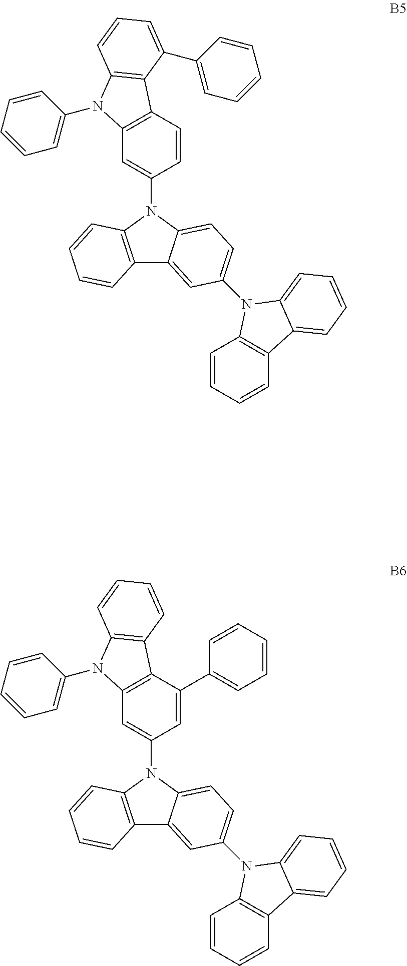 [SMILES: c1ccc(-c2cccc3c2c2ccc(-n4c5ccccc5c5cc(-n6c7ccccc7c7ccccc76)ccc54)cc2n3-c2ccccc2)cc1 ; c1ccc(-c2cc(-n3c4ccccc4c4cc(-n5c6ccccc6c6ccccc65)ccc43)cc3c2c2ccccc2n3-c2ccccc2)cc1]

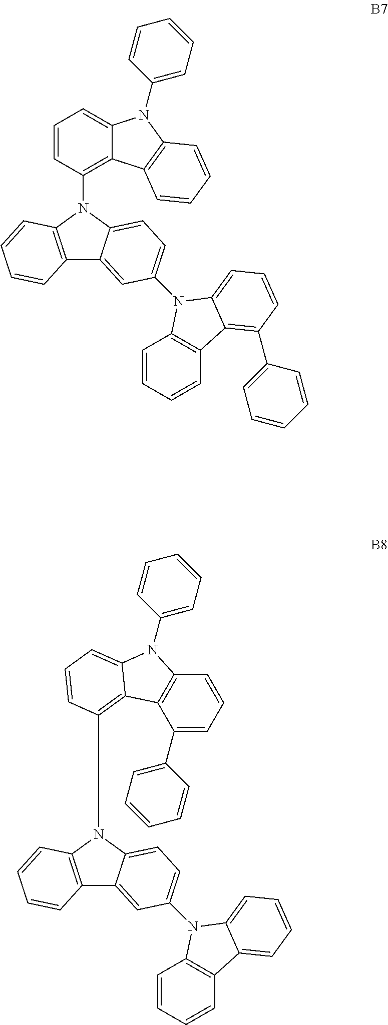 [SMILES: c1ccc(-c2cccc3c2c2ccccc2n3-c2ccc3c(c2)c2ccccc2n3-c2cccc3c2c2ccccc2n3-c2ccccc2)cc1 ; c1ccc(-c2cccc3c2c2c(-n4c5ccccc5c5cc(-n6c7ccccc7c7ccccc76)ccc54)cccc2n3-c2ccccc2)cc1]

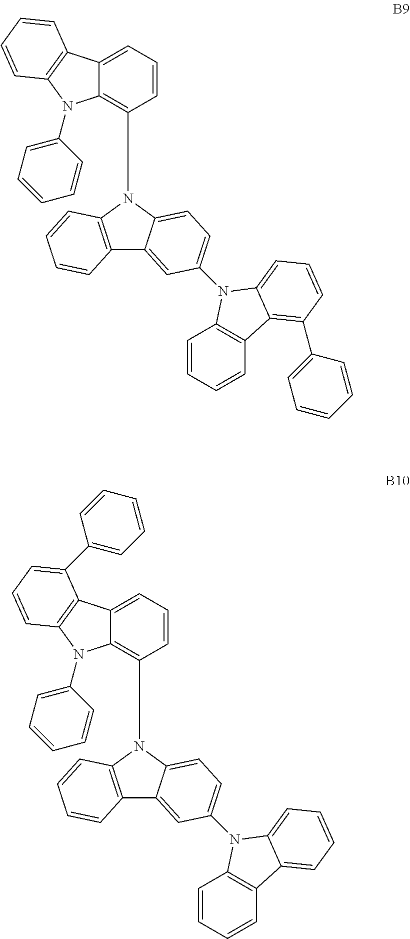 [SMILES: c1ccc(-c2cccc3c2c2ccccc2n3-c2ccc3c(c2)c2ccccc2n3-c2cccc3c4ccccc4n(-c4ccccc4)c23)cc1 ; c1ccc(-c2cccc3c2c2cccc(-n4c5ccccc5c5cc(-n6c7ccccc7c7ccccc76)ccc54)c2n3-c2ccccc2)cc1]

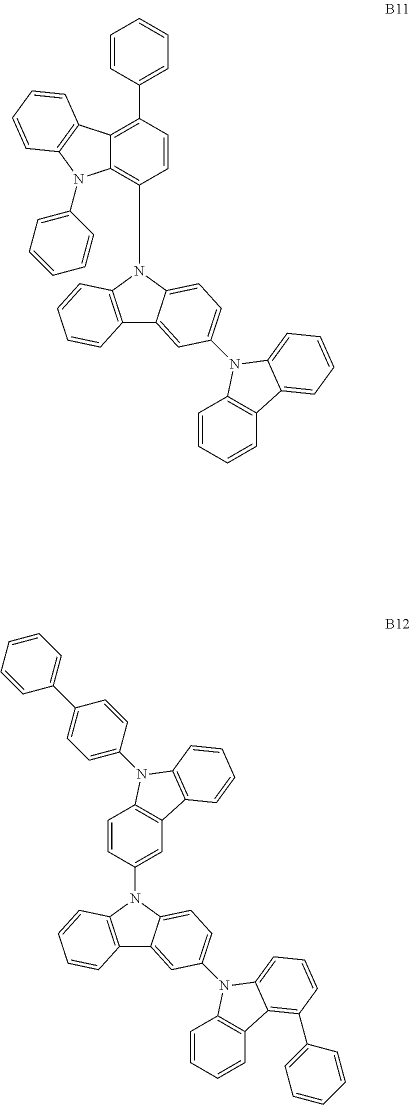 [SMILES: c1ccc(-c2ccc(-n3c4ccccc4c4cc(-n5c6ccccc6c6ccccc65)ccc43)c3c2c2ccccc2n3-c2ccccc2)cc1 ; c1ccc(-c2ccc(-n3c4ccccc4c4cc(-n5c6ccccc6c6cc(-n7c8ccccc8c8c(-c9ccccc9)cccc87)ccc65)ccc43)cc2)cc1]

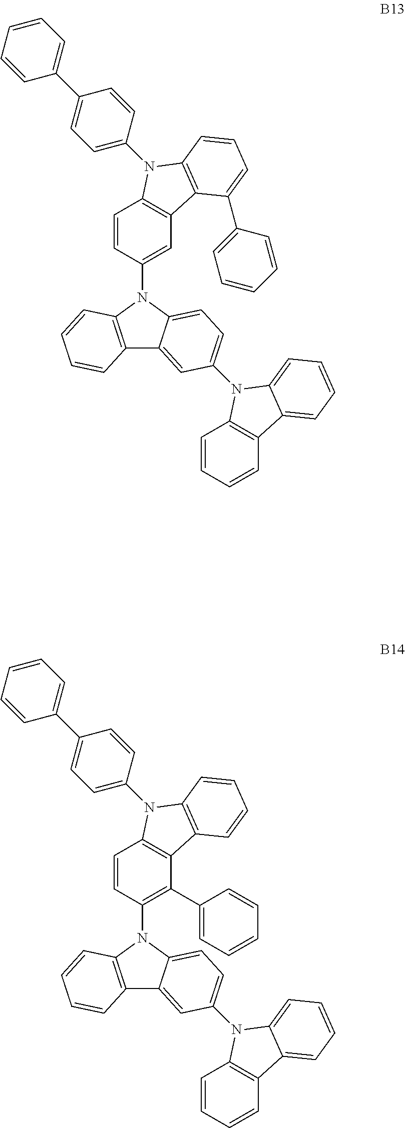 [SMILES: c1ccc(-c2ccc(-n3c4ccc(-n5c6ccccc6c6cc(-n7c8ccccc8c8ccccc87)ccc65)cc4c4c(-c5ccccc5)cccc43)cc2)cc1 ; c1ccc(-c2ccc(-n3c4ccccc4c4c(-c5ccccc5)c(-n5c6ccccc6c6cc(-n7c8ccccc8c8ccccc87)ccc65)ccc43)cc2)cc1]

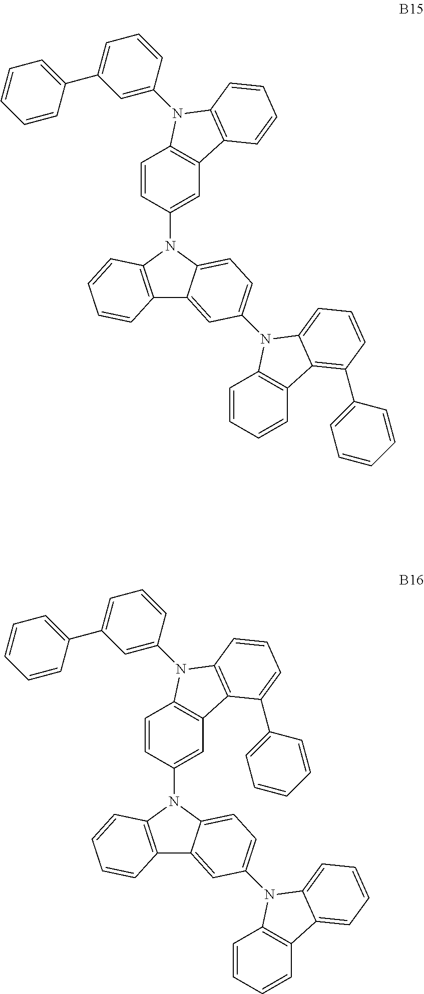 [SMILES: c1ccc(-c2cccc(-n3c4ccccc4c4cc(-n5c6ccccc6c6cc(-n7c8ccccc8c8c(-c9ccccc9)cccc87)ccc65)ccc43)c2)cc1 ; c1ccc(-c2cccc(-n3c4ccc(-n5c6ccccc6c6cc(-n7c8ccccc8c8ccccc87)ccc65)cc4c4c(-c5ccccc5)cccc43)c2)cc1]

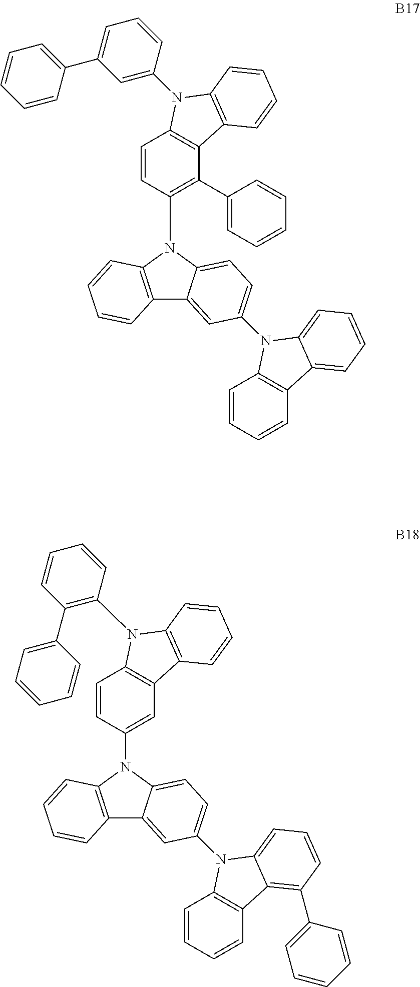 [SMILES: c1ccc(-c2cccc(-n3c4ccccc4c4c(-c5ccccc5)c(-n5c6ccccc6c6cc(-n7c8ccccc8c8ccccc87)ccc65)ccc43)c2)cc1 ; c1ccc(-c2ccccc2-n2c3ccccc3c3cc(-n4c5ccccc5c5cc(-n6c7ccccc7c7c(-c8ccccc8)cccc76)ccc54)ccc32)cc1]

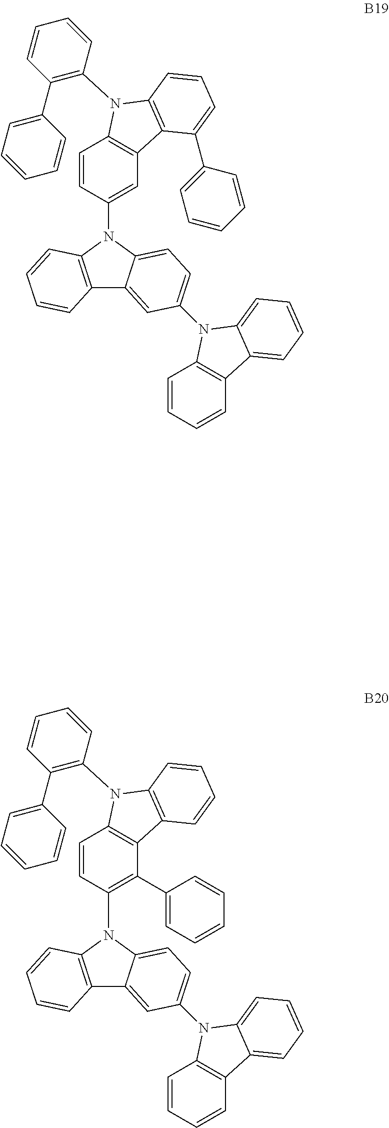 [SMILES: c1ccc(-c2ccccc2-n2c3ccc(-n4c5ccccc5c5cc(-n6c7ccccc7c7ccccc76)ccc54)cc3c3c(-c4ccccc4)cccc32)cc1 ; c1ccc(-c2ccccc2-n2c3ccccc3c3c(-c4ccccc4)c(-n4c5ccccc5c5cc(-n6c7ccccc7c7ccccc76)ccc54)ccc32)cc1]

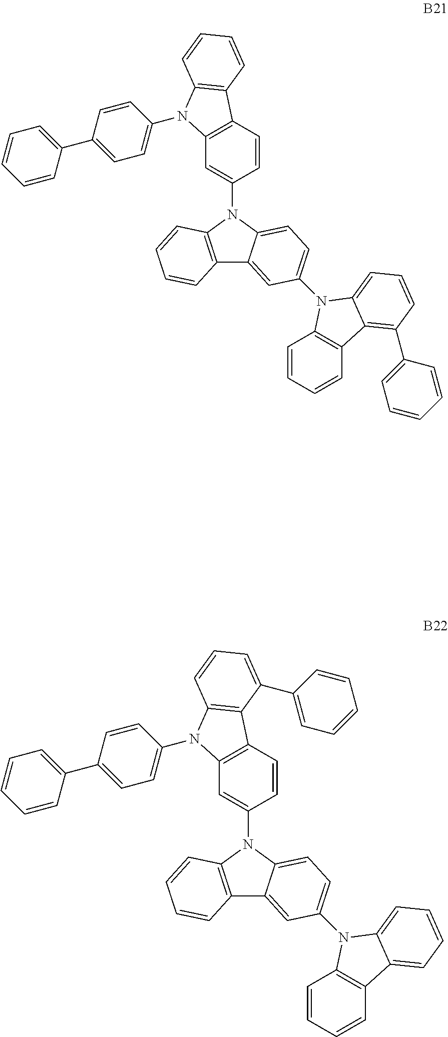 [SMILES: c1ccc(-c2ccc(-n3c4ccccc4c4ccc(-n5c6ccccc6c6cc(-n7c8ccccc8c8c(-c9ccccc9)cccc87)ccc65)cc43)cc2)cc1 ; c1ccc(-c2ccc(-n3c4cc(-n5c6ccccc6c6cc(-n7c8ccccc8c8ccccc87)ccc65)ccc4c4c(-c5ccccc5)cccc43)cc2)cc1]

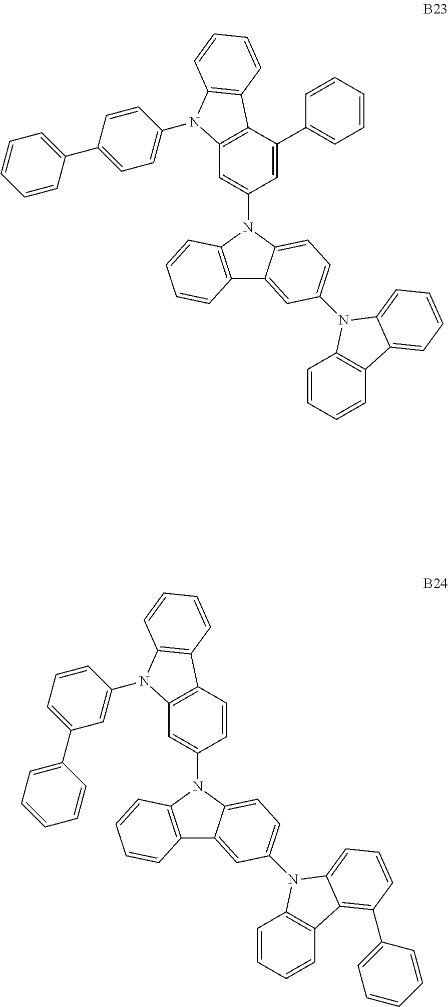 [SMILES: c1ccc(-c2ccc(-n3c4ccccc4c4c(-c5ccccc5)cc(-n5c6ccccc6c6cc(-n7c8ccccc8c8ccccc87)ccc65)cc43)cc2)cc1 ; c1ccc(-c2cccc(-n3c4ccccc4c4ccc(-n5c6ccccc6c6cc(-n7c8ccccc8c8c(-c9ccccc9)cccc87)ccc65)cc43)c2)cc1]

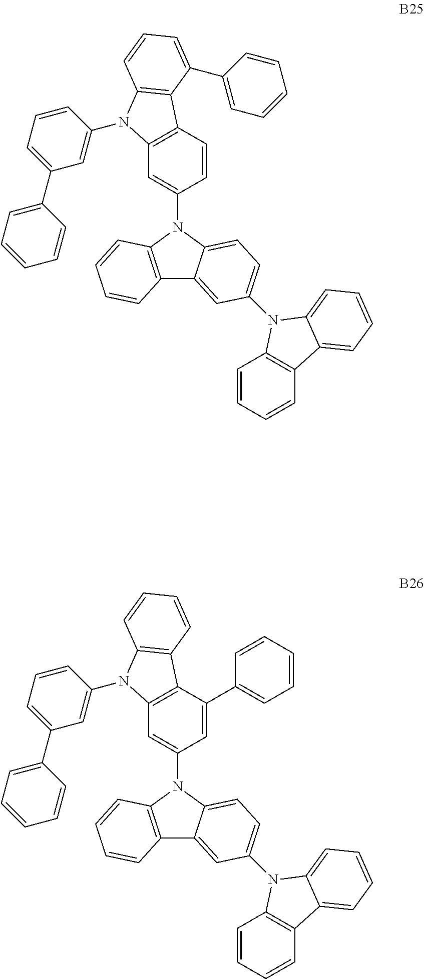 [SMILES: c1ccc(-c2cccc(-n3c4cc(-n5c6ccccc6c6cc(-n7c8ccccc8c8ccccc87)ccc65)ccc4c4c(-c5ccccc5)cccc43)c2)cc1 ; c1ccc(-c2cccc(-n3c4ccccc4c4c(-c5ccccc5)cc(-n5c6ccccc6c6cc(-n7c8ccccc8c8ccccc87)ccc65)cc43)c2)cc1]

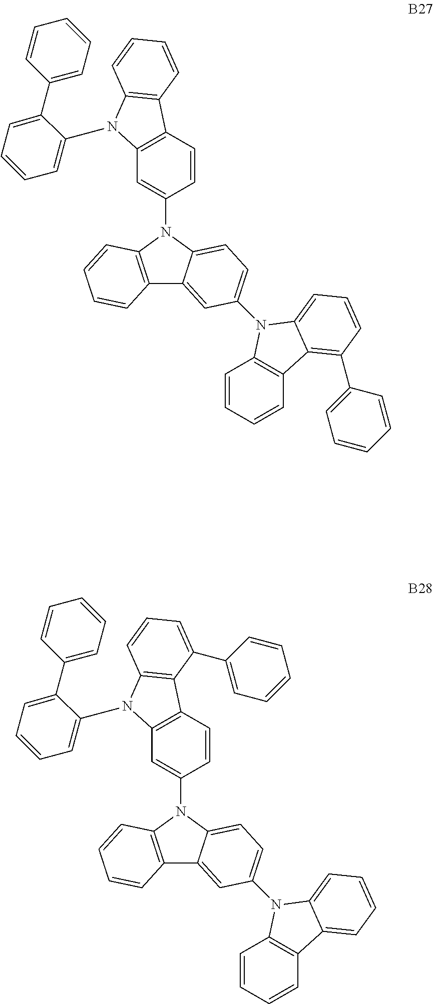 [SMILES: c1ccc(-c2ccccc2-n2c3ccccc3c3ccc(-n4c5ccccc5c5cc(-n6c7ccccc7c7c(-c8ccccc8)cccc76)ccc54)cc32)cc1 ; c1ccc(-c2ccccc2-n2c3cc(-n4c5ccccc5c5cc(-n6c7ccccc7c7ccccc76)ccc54)ccc3c3c(-c4ccccc4)cccc32)cc1]

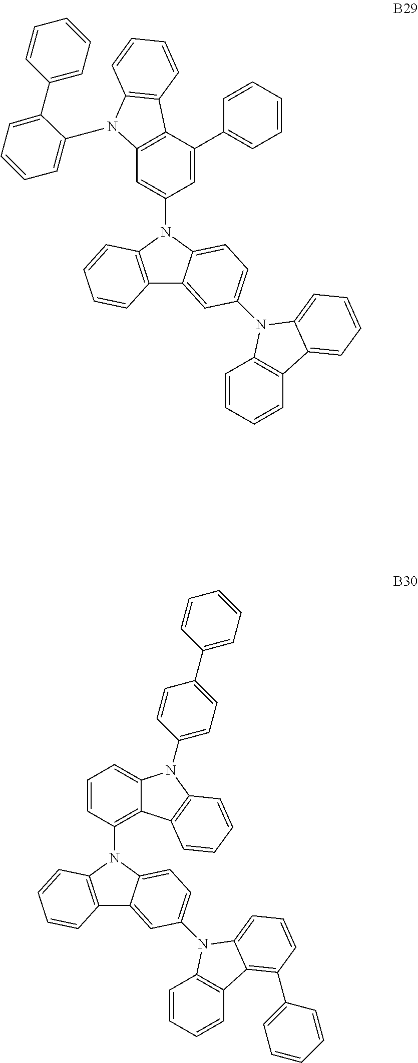 [SMILES: c1ccc(-c2ccccc2-n2c3ccccc3c3c(-c4ccccc4)cc(-n4c5ccccc5c5cc(-n6c7ccccc7c7ccccc76)ccc54)cc32)cc1 ; c1ccc(-c2ccc(-n3c4ccccc4c4c(-n5c6ccccc6c6cc(-n7c8ccccc8c8c(-c9ccccc9)cccc87)ccc65)cccc43)cc2)cc1]

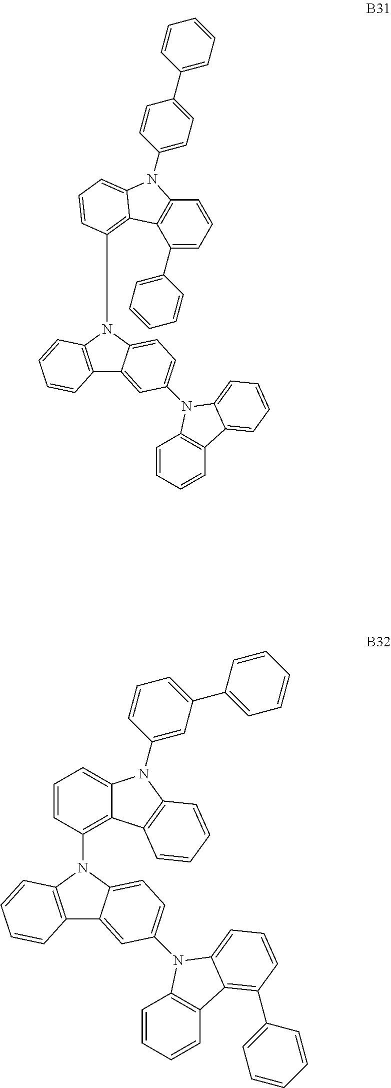 [SMILES: c1ccc(-c2ccc(-n3c4cccc(-c5ccccc5)c4c4c(-n5c6ccccc6c6cc(-n7c8ccccc8c8ccccc87)ccc65)cccc43)cc2)cc1 ; c1ccc(-c2cccc(-n3c4ccccc4c4c(-n5c6ccccc6c6cc(-n7c8ccccc8c8c(-c9ccccc9)cccc87)ccc65)cccc43)c2)cc1]

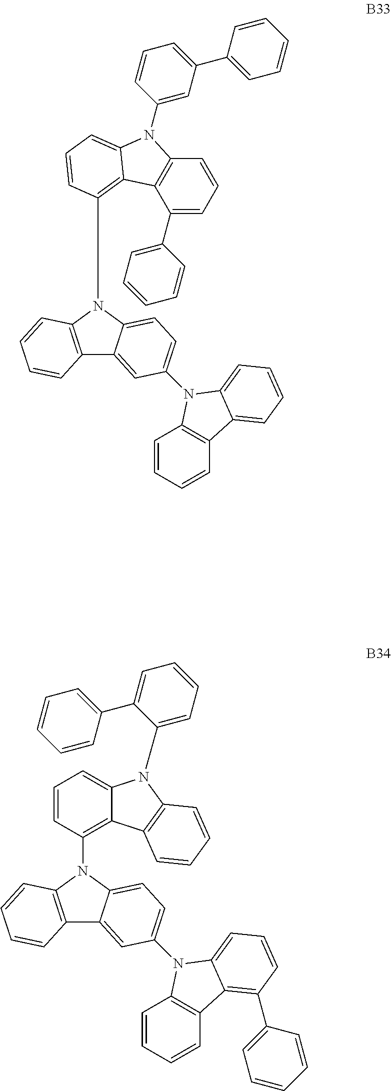 [SMILES: c1ccc(-c2cccc(-n3c4cccc(-c5ccccc5)c4c4c(-n5c6ccccc6c6cc(-n7c8ccccc8c8ccccc87)ccc65)cccc43)c2)cc1 ; c1ccc(-c2ccccc2-n2c3ccccc3c3c(-n4c5ccccc5c5cc(-n6c7ccccc7c7c(-c8ccccc8)cccc76)ccc54)cccc32)cc1]

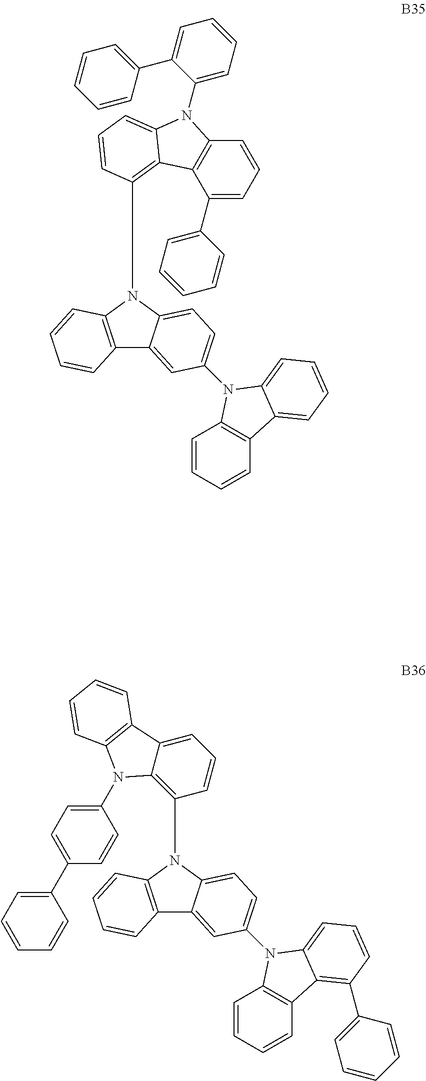 [SMILES: c1ccc(-c2ccccc2-n2c3cccc(-c4ccccc4)c3c3c(-n4c5ccccc5c5cc(-n6c7ccccc7c7ccccc76)ccc54)cccc32)cc1 ; c1ccc(-c2ccc(-n3c4ccccc4c4cccc(-n5c6ccccc6c6cc(-n7c8ccccc8c8c(-c9ccccc9)cccc87)ccc65)c43)cc2)cc1]

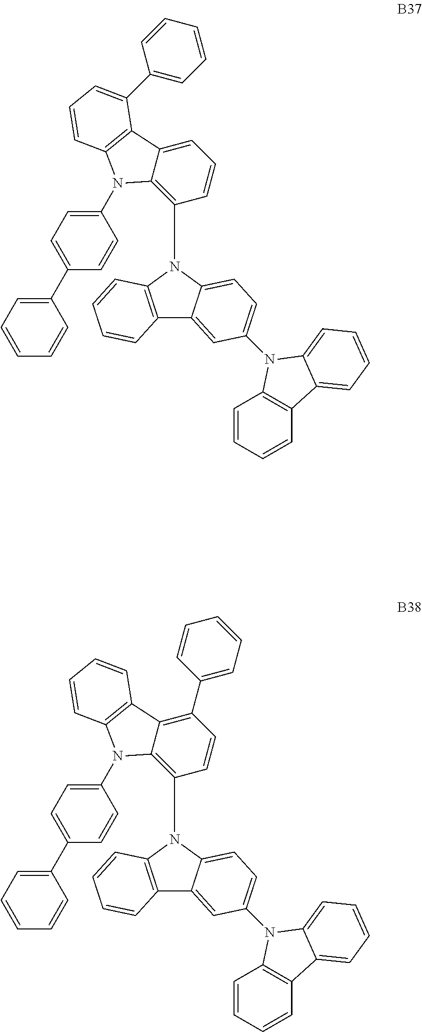 [SMILES: c1ccc(-c2ccc(-n3c4cccc(-c5ccccc5)c4c4cccc(-n5c6ccccc6c6cc(-n7c8ccccc8c8ccccc87)ccc65)c43)cc2)cc1 ; c1ccc(-c2ccc(-n3c4ccccc4c4c(-c5ccccc5)ccc(-n5c6ccccc6c6cc(-n7c8ccccc8c8ccccc87)ccc65)c43)cc2)cc1]

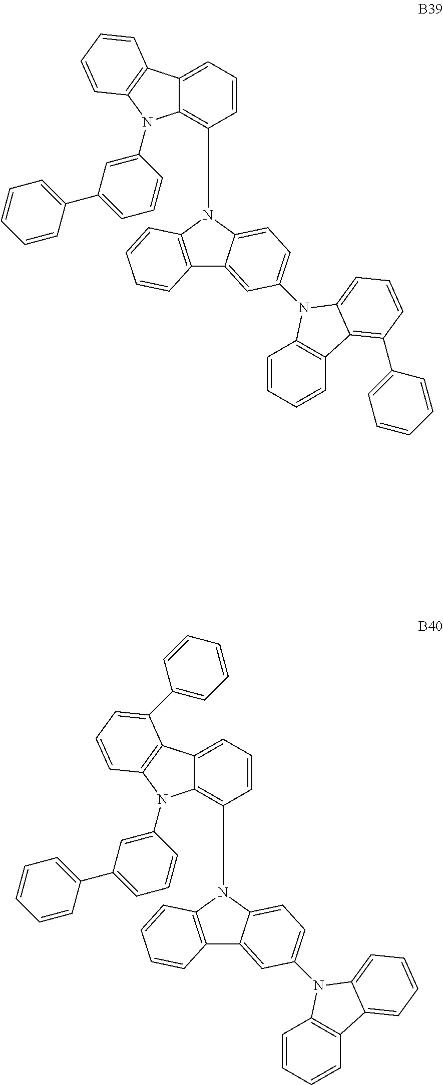 [SMILES: c1ccc(-c2cccc(-n3c4ccccc4c4cccc(-n5c6ccccc6c6cc(-n7c8ccccc8c8c(-c9ccccc9)cccc87)ccc65)c43)c2)cc1 ; c1ccc(-c2cccc(-n3c4cccc(-c5ccccc5)c4c4cccc(-n5c6ccccc6c6cc(-n7c8ccccc8c8ccccc87)ccc65)c43)c2)cc1]

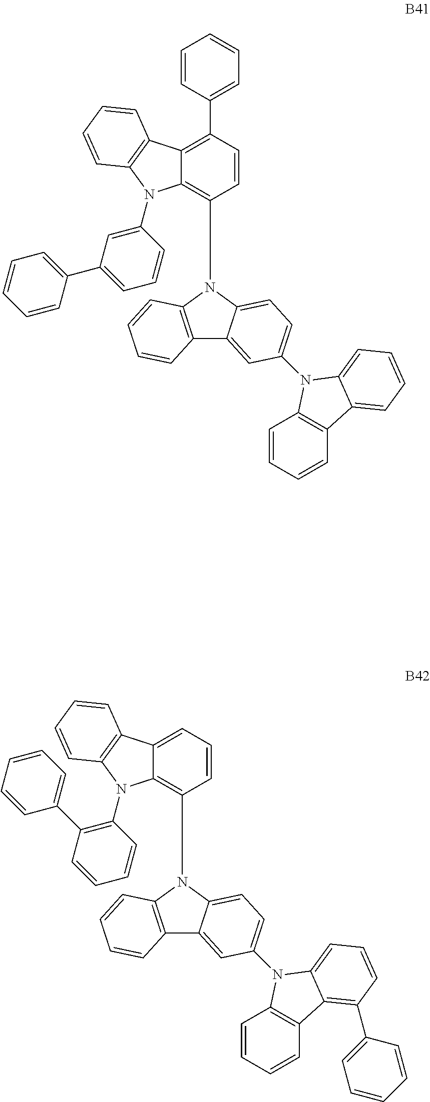 [SMILES: c1ccc(-c2cccc(-n3c4ccccc4c4c(-c5ccccc5)ccc(-n5c6ccccc6c6cc(-n7c8ccccc8c8ccccc87)ccc65)c43)c2)cc1 ; c1ccc(-c2ccccc2-n2c3ccccc3c3cccc(-n4c5ccccc5c5cc(-n6c7ccccc7c7c(-c8ccccc8)cccc76)ccc54)c32)cc1]

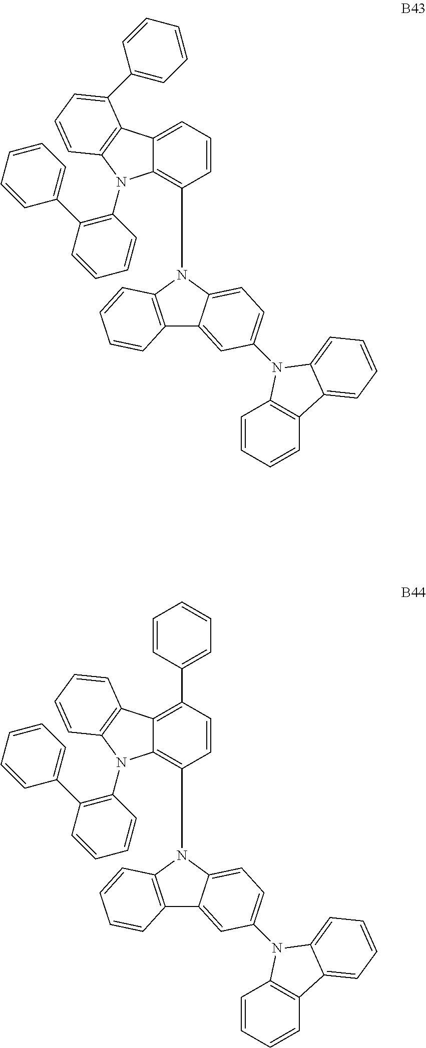 [SMILES: c1ccc(-c2ccccc2-n2c3cccc(-c4ccccc4)c3c3cccc(-n4c5ccccc5c5cc(-n6c7ccccc7c7ccccc76)ccc54)c32)cc1 ; c1ccc(-c2ccccc2-n2c3ccccc3c3c(-c4ccccc4)ccc(-n4c5ccccc5c5cc(-n6c7ccccc7c7ccccc76)ccc54)c32)cc1]

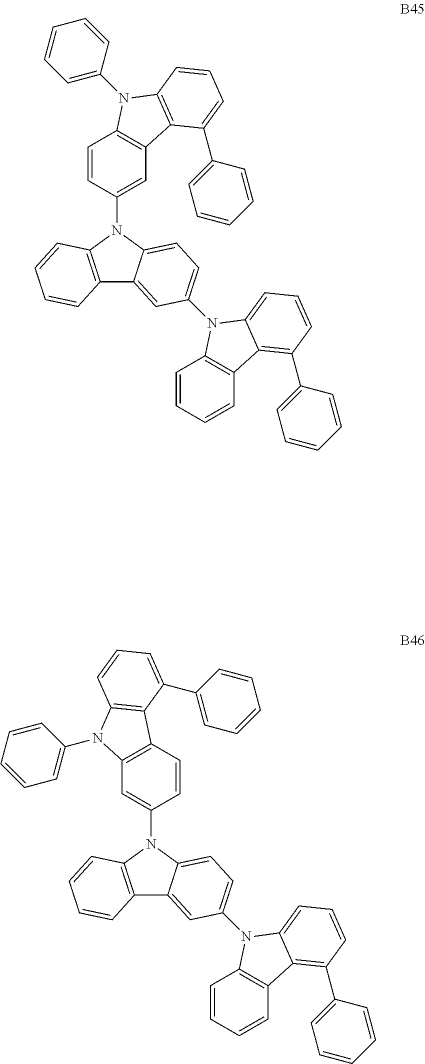 [SMILES: c1ccc(-c2cccc3c2c2ccccc2n3-c2ccc3c(c2)c2ccccc2n3-c2ccc3c(c2)c2c(-c4ccccc4)cccc2n3-c2ccccc2)cc1 ; c1ccc(-c2cccc3c2c2ccccc2n3-c2ccc3c(c2)c2ccccc2n3-c2ccc3c4c(-c5ccccc5)cccc4n(-c4ccccc4)c3c2)cc1]

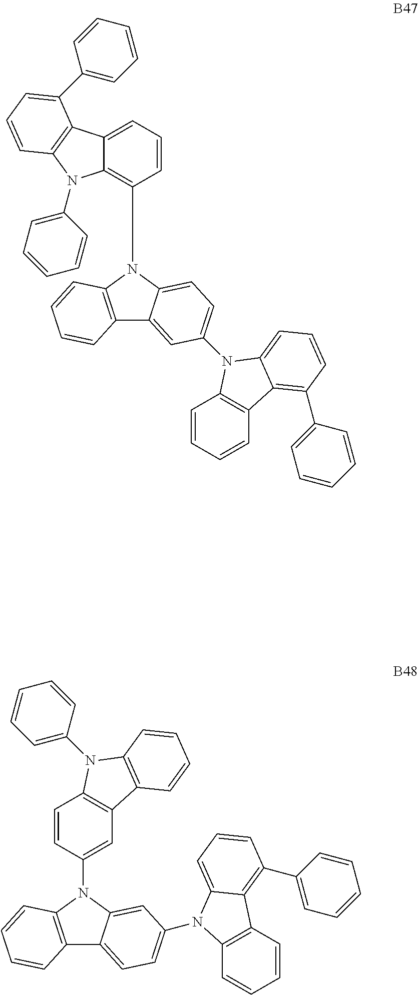 [SMILES: c1ccc(-c2cccc3c2c2ccccc2n3-c2ccc3c(c2)c2ccccc2n3-c2cccc3c4c(-c5ccccc5)cccc4n(-c4ccccc4)c23)cc1 ; c1ccc(-c2cccc3c2c2ccccc2n3-c2ccc3c4ccccc4n(-c4ccc5c(c4)c4ccccc4n5-c4ccccc4)c3c2)cc1]

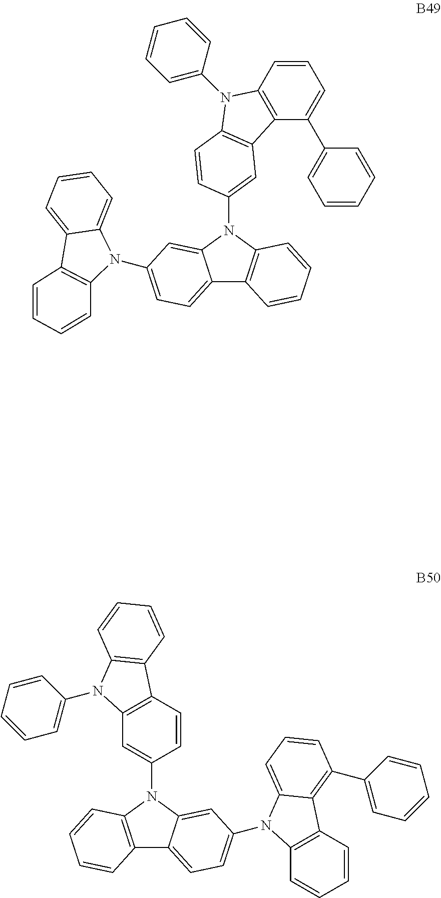 [SMILES: c1ccc(-c2cccc3c2c2cc(-n4c5ccccc5c5ccc(-n6c7ccccc7c7ccccc76)cc54)ccc2n3-c2ccccc2)cc1 ; c1ccc(-c2cccc3c2c2ccccc2n3-c2ccc3c4ccccc4n(-c4ccc5c6ccccc6n(-c6ccccc6)c5c4)c3c2)cc1]

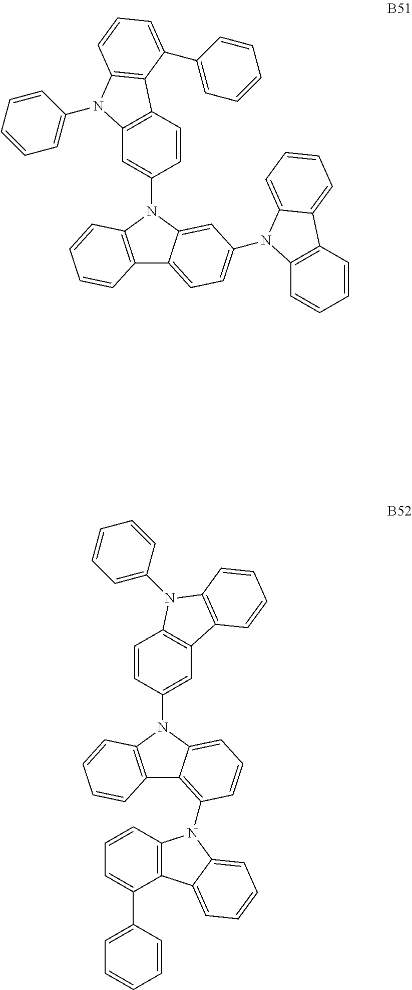 [SMILES: c1ccc(-c2cccc3c2c2ccc(-n4c5ccccc5c5ccc(-n6c7ccccc7c7ccccc76)cc54)cc2n3-c2ccccc2)cc1 ; c1ccc(-c2cccc3c2c2ccccc2n3-c2cccc3c2c2ccccc2n3-c2ccc3c(c2)c2ccccc2n3-c2ccccc2)cc1]

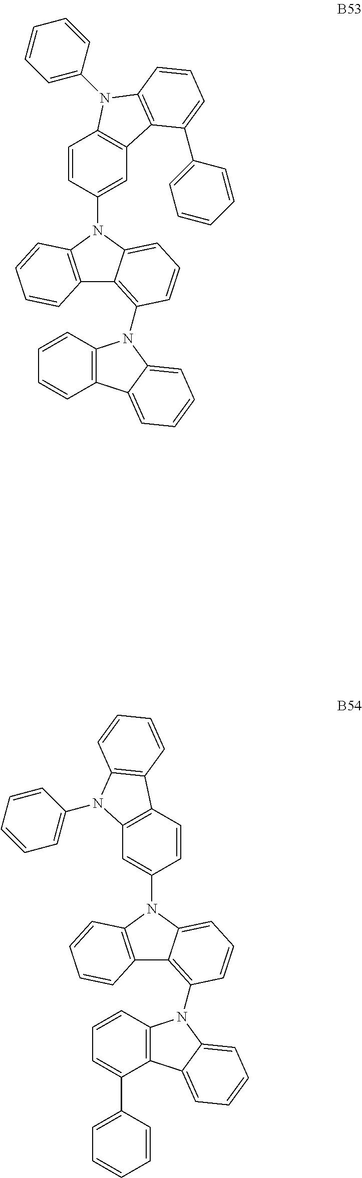 [SMILES: c1ccc(-c2cccc3c2c2cc(-n4c5ccccc5c5c(-n6c7ccccc7c7ccccc76)cccc54)ccc2n3-c2ccccc2)cc1 ; c1ccc(-c2cccc3c2c2ccccc2n3-c2cccc3c2c2ccccc2n3-c2ccc3c4ccccc4n(-c4ccccc4)c3c2)cc1]

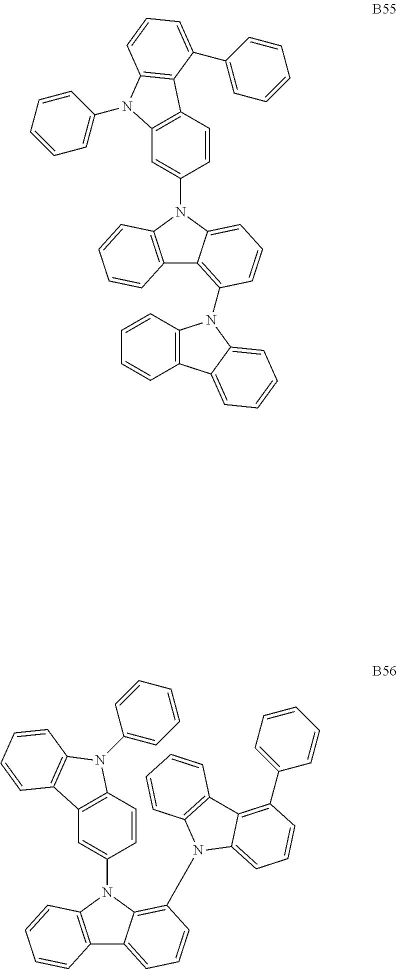 [SMILES: c1ccc(-c2cccc3c2c2ccc(-n4c5ccccc5c5c(-n6c7ccccc7c7ccccc76)cccc54)cc2n3-c2ccccc2)cc1 ; c1ccc(-c2cccc3c2c2ccccc2n3-c2cccc3c4ccccc4n(-c4ccc5c(c4)c4ccccc4n5-c4ccccc4)c23)cc1]

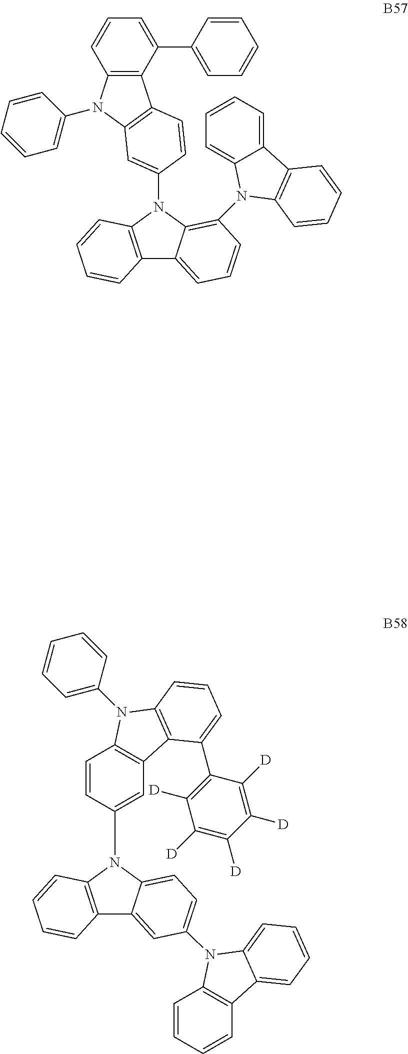 [SMILES: c1ccc(-c2cccc3c2c2ccc(-n4c5ccccc5c5cccc(-n6c7ccccc7c7ccccc76)c54)cc2n3-c2ccccc2)cc1 ; [2H]c1c([2H])c([2H])c(-c2cccc3c2c2cc(-n4c5ccccc5c5cc(-n6c7ccccc7c7ccccc76)ccc54)ccc2n3-c2ccccc2)c([2H])c1[2H]]

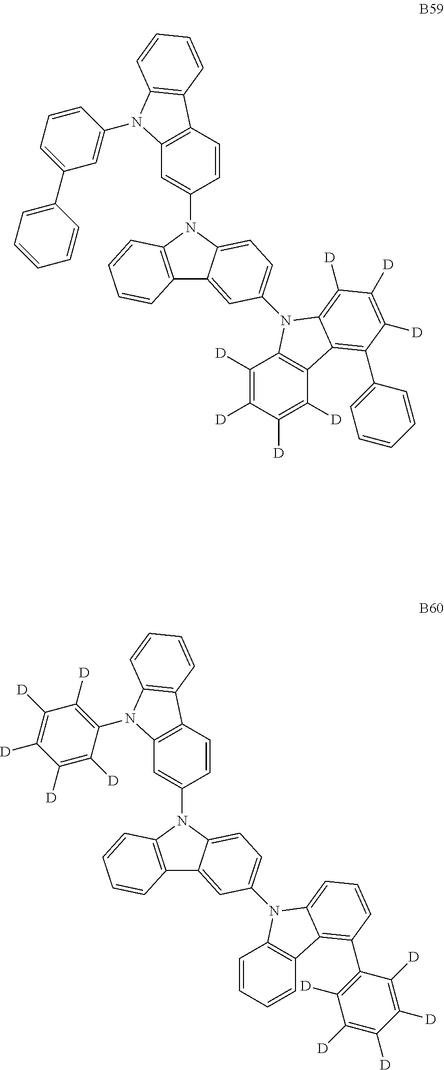 [SMILES: [2H]c1c([2H])c([2H])c2c(c1[2H])c1c(-c3ccccc3)c([2H])c([2H])c([2H])c1n2-c1ccc2c(c1)c1ccccc1n2-c1ccc2c3ccccc3n(-c3cccc(-c4ccccc4)c3)c2c1 ; [2H]c1c([2H])c([2H])c(-c2cccc3c2c2ccccc2n3-c2ccc3c(c2)c2ccccc2n3-c2ccc3c4ccccc4n(-c4c([2H])c([2H])c([2H])c([2H])c4[2H])c3c2)c([2H])c1[2H]]

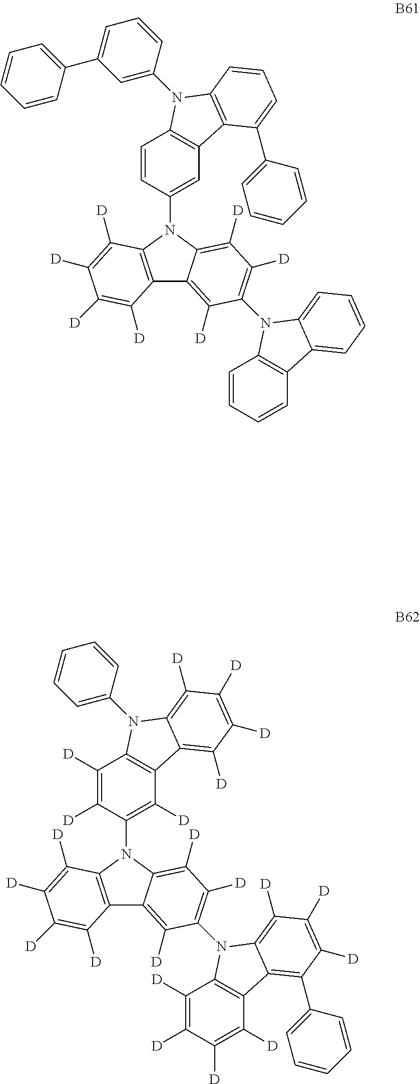 [SMILES: [2H]c1c([2H])c([2H])c2c(c1[2H])c1c([2H])c(-n3c4ccccc4c4ccccc43)c([2H])c([2H])c1n2-c1ccc2c(c1)c1c(-c3ccccc3)cccc1n2-c1cccc(-c2ccccc2)c1 ; [2H]c1c([2H])c([2H])c2c(c1[2H])c1c([2H])c(-n3c4c([2H])c([2H])c([2H])c([2H])c4c4c([2H])c(-n5c6c([2H])c([2H])c([2H])c([2H])c6c6c(-c7ccccc7)c([2H])c([2H])c([2H])c65)c([2H])c([2H])c43)c([2H])c([2H])c1n2-c1ccccc1]

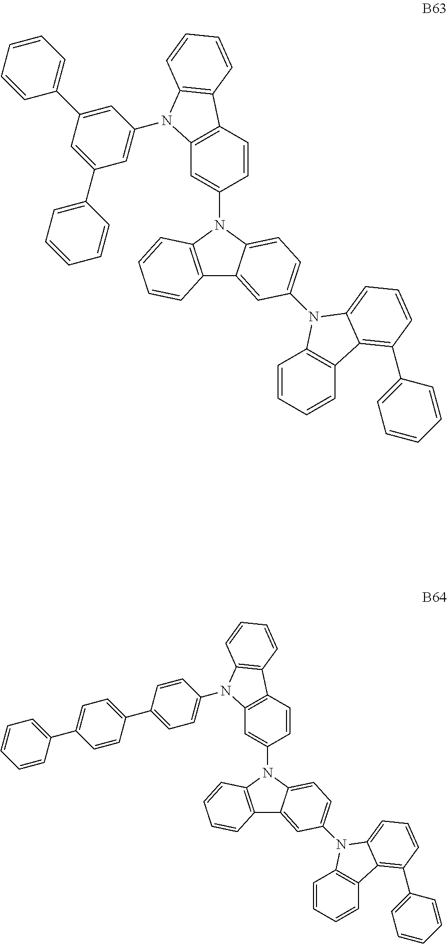 [SMILES: c1ccc(-c2cc(-c3ccccc3)cc(-n3c4ccccc4c4ccc(-n5c6ccccc6c6cc(-n7c8ccccc8c8c(-c9ccccc9)cccc87)ccc65)cc43)c2)cc1 ; c1ccc(-c2ccc(-c3ccc(-n4c5ccccc5c5ccc(-n6c7ccccc7c7cc(-n8c9ccccc9c9c(-c%10ccccc%10)cccc98)ccc76)cc54)cc3)cc2)cc1]

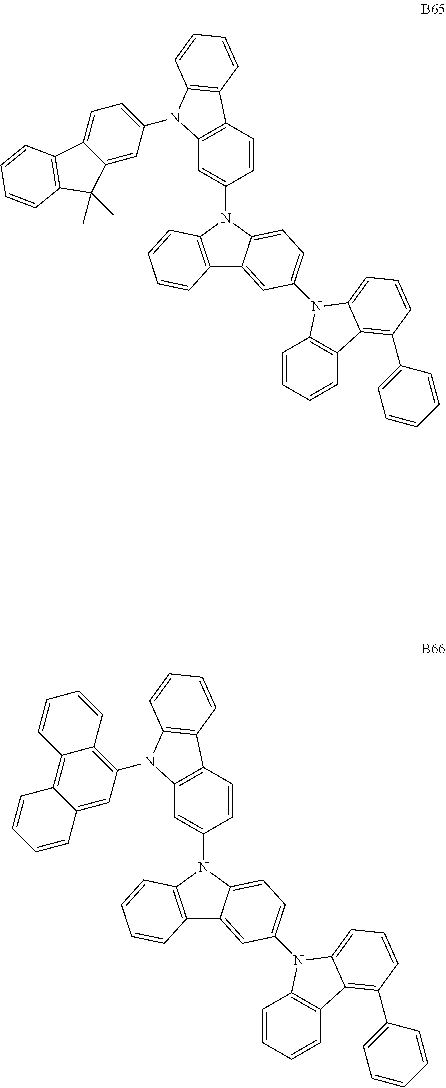 [SMILES: CC1(C)c2ccccc2-c2ccc(-n3c4ccccc4c4ccc(-n5c6ccccc6c6cc(-n7c8ccccc8c8c(-c9ccccc9)cccc87)ccc65)cc43)cc21 ; c1ccc(-c2cccc3c2c2ccccc2n3-c2ccc3c(c2)c2ccccc2n3-c2ccc3c4ccccc4n(-c4cc5ccccc5c5ccccc45)c3c2)cc1]

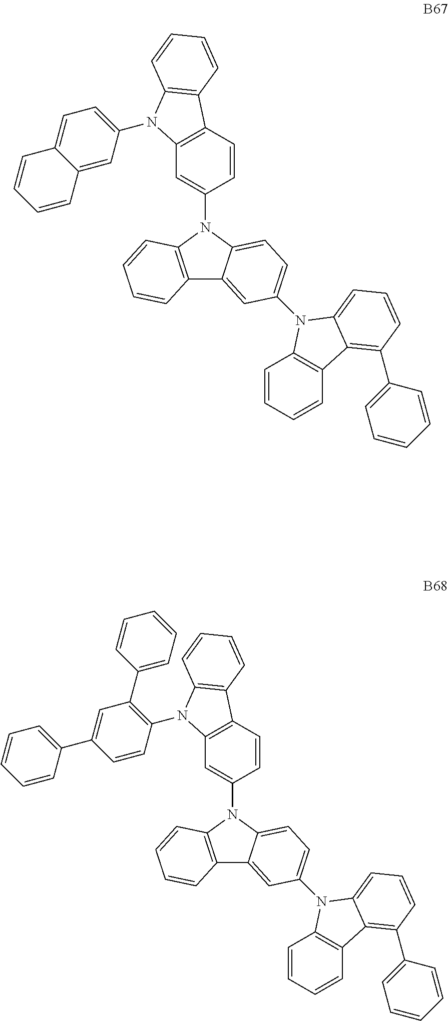 [SMILES: c1ccc(-c2cccc3c2c2ccccc2n3-c2ccc3c(c2)c2ccccc2n3-c2ccc3c4ccccc4n(-c4ccc5ccccc5c4)c3c2)cc1 ; c1ccc(-c2ccc(-n3c4ccccc4c4ccc(-n5c6ccccc6c6cc(-n7c8ccccc8c8c(-c9ccccc9)cccc87)ccc65)cc43)c(-c3ccccc3)c2)cc1]

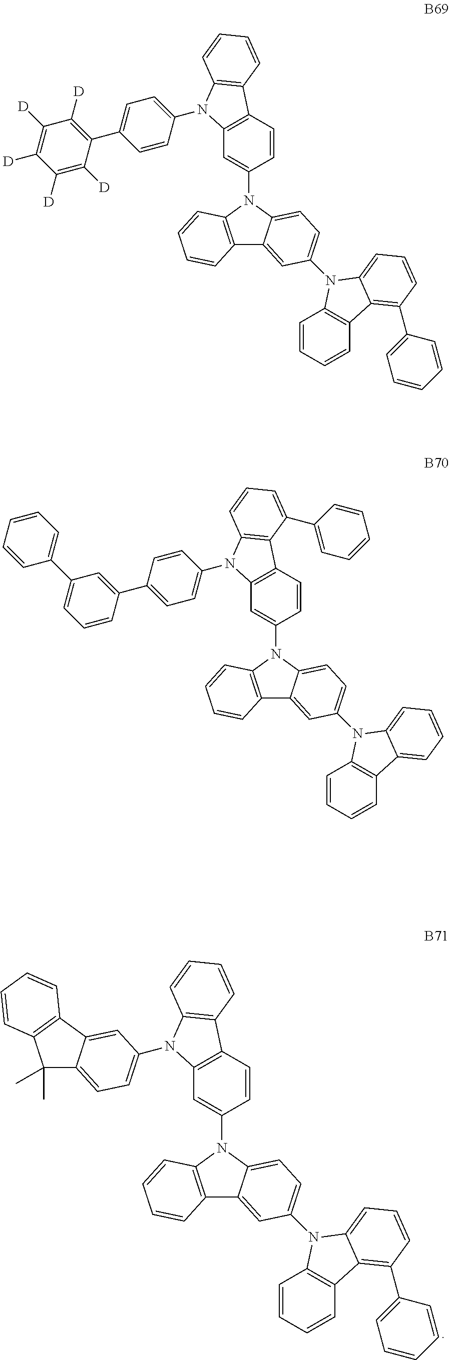 [SMILES: [2H]c1c([2H])c([2H])c(-c2ccc(-n3c4ccccc4c4ccc(-n5c6ccccc6c6cc(-n7c8ccccc8c8c(-c9ccccc9)cccc87)ccc65)cc43)cc2)c([2H])c1[2H] ; c1ccc(-c2cccc(-c3ccc(-n4c5cc(-n6c7ccccc7c7cc(-n8c9ccccc9c9ccccc98)ccc76)ccc5c5c(-c6ccccc6)cccc54)cc3)c2)cc1 ; CC1(C)c2ccccc2-c2cc(-n3c4ccccc4c4ccc(-n5c6ccccc6c6cc(-n7c8ccccc8c8c(-c9ccccc9)cccc87)ccc65)cc43)ccc21]

A second aspect of the present disclosure provides an organic electroluminescent device, including an anode, a cathode, and a functional layer disposed between the anode and the cathode, where the functional layer includes the nitrogen-containing compound according to the first aspect of the present disclosure.

The nitrogen-containing compound provided in the present disclosure can be used for forming at least one organic film layer in the functional layer so as to improve the characteristics such as the luminous efficiency and service life of the organic electroluminescent device.

Optionally, the functional layer includes an organic light-emitting layer including the nitrogen-containing compound. The organic light-emitting layer may be composed of the nitrogen-containing compound provided by the present disclosure, and may also be composed of the nitrogen-containing compound provided by the present disclosure and other materials together.

Optionally, the functional layer further includes a hole transport layer, and the hole transport layer is located between the anode and the organic light-emitting layer.

In a specific embodiment of the present disclosure, the organic electroluminescent device is a blue phosphorescent organic electroluminescent device.

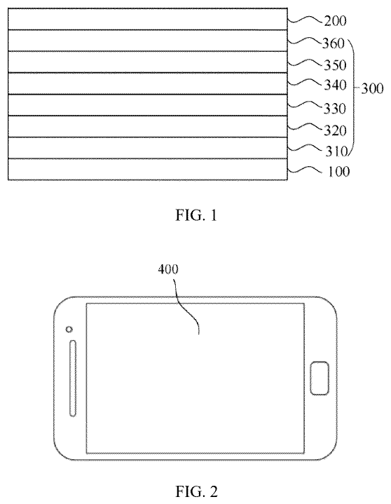

According to a specific embodiment, the organic electroluminescent device is shown in FIG. 1, and the organic electroluminescent device may include an anode 100, a hole injection layer 310, a hole transport layer 320, an electron blocking layer 330, an organic light-emitting layer 340, an electron transport layer 350, an electron injection layer 360, and a cathode 200 which are sequentially stacked.

Optionally, the anode 100 includes the following anode materials, which are preferably materials having a large work function that facilitate hole injection into the functional layer. Specific examples of the anode materials include metals such as nickel, platinum, vanadium, chromium, copper, zinc, and gold, or its alloy; metal oxides such as zinc oxide, indium oxide, indium tin oxide (ITO) and indium zinc oxide (IZO); combined metals and oxides, such as ZnO: Al or SnO₂: Sb; or a conductive polymer such as poly(3-methylthiophene), poly[3,4-(ethylene-1,2-dioxy)thiophene] (PEDT), polypyrrole, and polyaniline, but are not limited to this. The anode 100 preferably includes a transparent electrode including indium tin oxide (ITO).

In the present disclosure, the hole transport layer may include one or more hole transport materials, and the hole transport layer materials may be selected from a carbazole polymer, carbazole connected triarylamine compounds or other types of compounds, and in particular may be selected from compounds shown below or any combination of them:

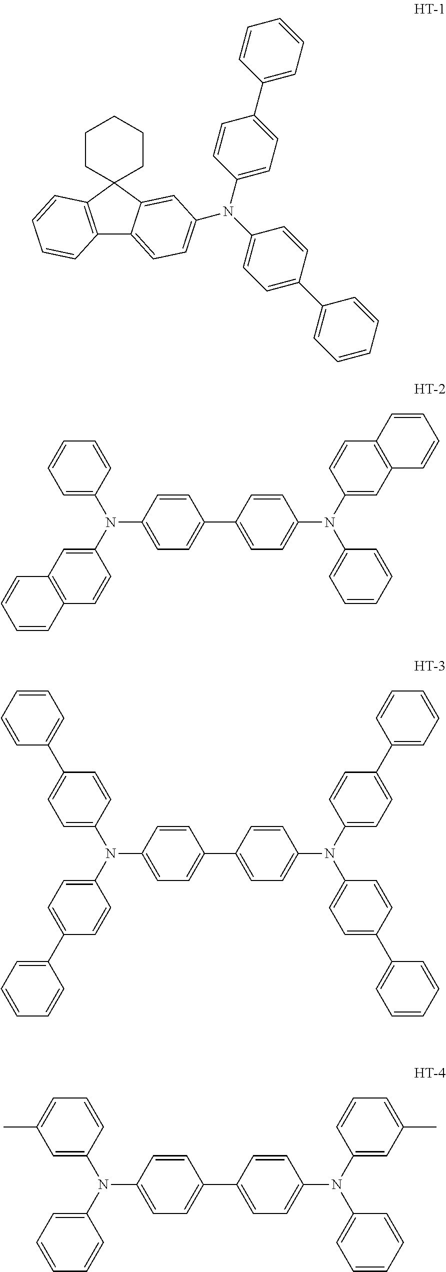

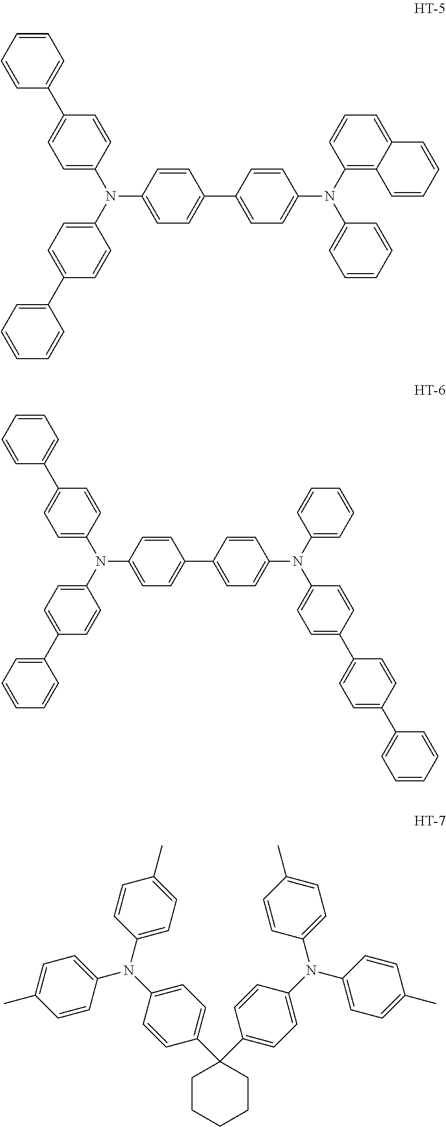

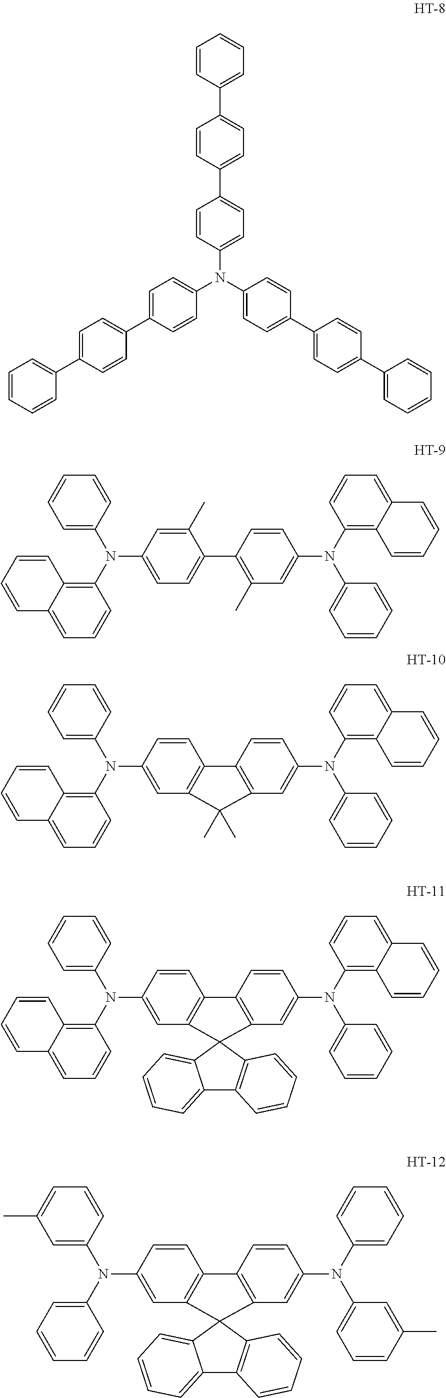

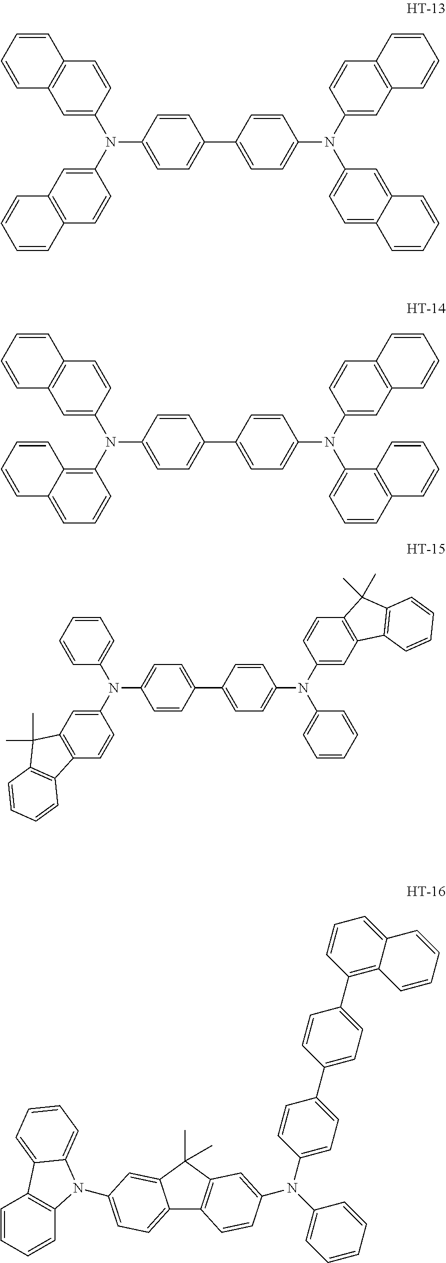 [SMILES: c1ccc2cc(N(c3ccc(-c4ccc(N(c5ccc6ccccc6c5)c5ccc6ccccc6c5)cc4)cc3)c3ccc4ccccc4c3)ccc2c1 ; c1ccc2cc(N(c3ccc(-c4ccc(N(c5ccc6ccccc6c5)c5cccc6ccccc56)cc4)cc3)c3cccc4ccccc34)ccc2c1 ; CC1(C)c2ccccc2-c2cc(N(c3ccccc3)c3ccc(-c4ccc(N(c5ccccc5)c5ccc6c(c5)C(C)(C)c5ccccc5-6)cc4)cc3)ccc21 ; CC1(C)c2cc(N(c3ccccc3)c3ccc(-c4ccc(-c5cccc6ccccc56)cc4)cc3)ccc2-c2ccc(-n3c4ccccc4c4ccccc43)cc21]

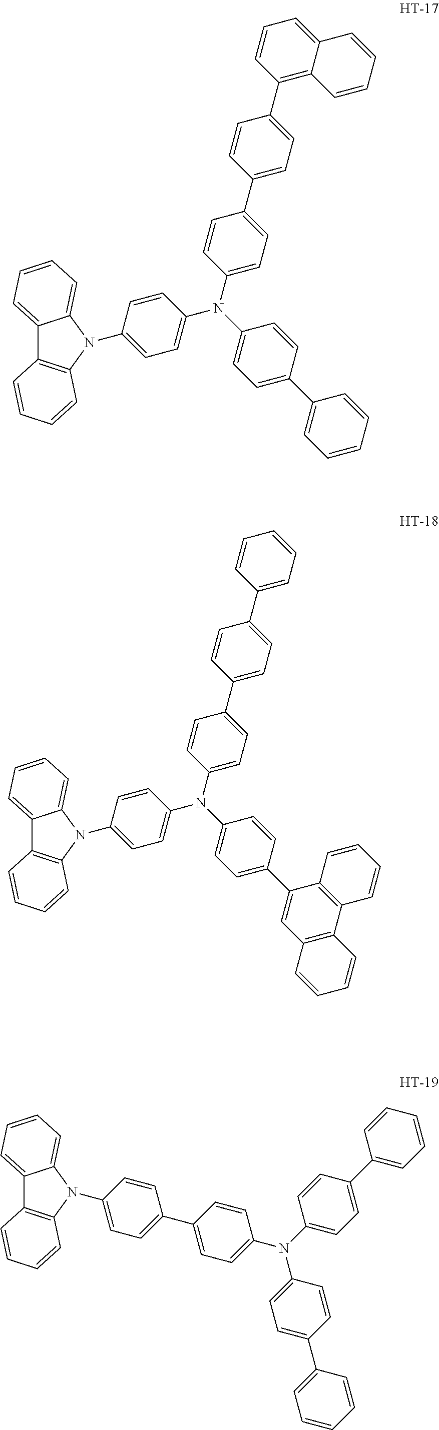 [SMILES: c1ccc(-c2ccc(N(c3ccc(-c4ccc(-c5cccc6ccccc56)cc4)cc3)c3ccc(-n4c5ccccc5c5ccccc54)cc3)cc2)cc1 ; c1ccc(-c2ccc(-c3ccc(N(c4ccc(-c5cc6ccccc6c6ccccc56)cc4)c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

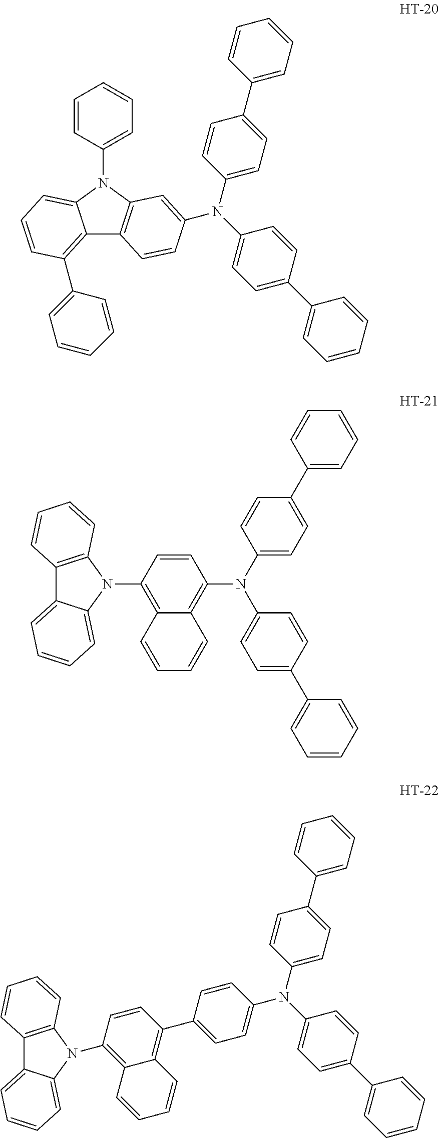 [SMILES: c1ccc(-c2ccc(N(c3ccc(-c4ccccc4)cc3)c3ccc4c5c(-c6ccccc6)cccc5n(-c5ccccc5)c4c3)cc2)cc1 ; c1ccc(-c2ccc(N(c3ccc(-c4ccccc4)cc3)c3ccc(-n4c5ccccc5c5ccccc54)c4ccccc34)cc2)cc1 ; c1ccc(-c2ccc(N(c3ccc(-c4ccccc4)cc3)c3ccc(-c4ccc(-n5c6ccccc6c6ccccc65)c5ccccc45)cc3)cc2)cc1]

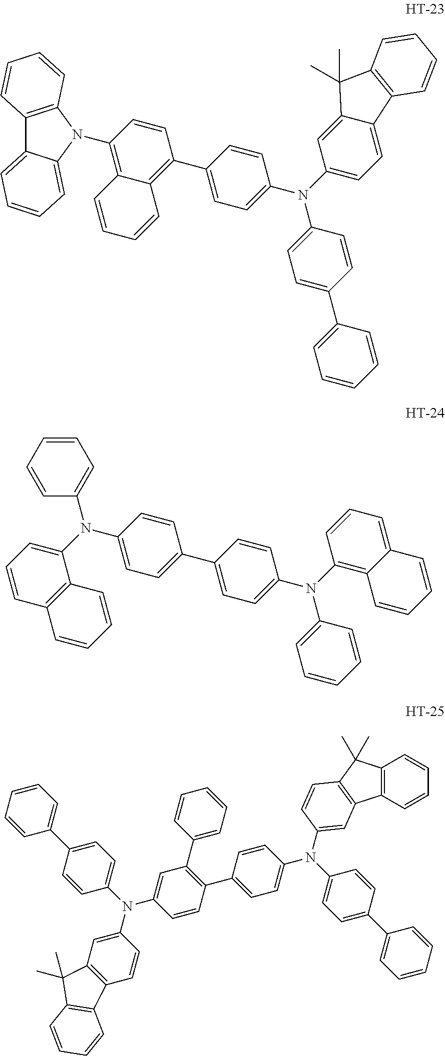 [SMILES: CC1(C)c2ccccc2-c2ccc(N(c3ccc(-c4ccccc4)cc3)c3ccc(-c4ccc(-n5c6ccccc6c6ccccc65)c5ccccc45)cc3)cc21 ; c1ccc(N(c2ccc(-c3ccc(N(c4ccccc4)c4cccc5ccccc45)cc3)cc2)c2cccc3ccccc23)cc1 ; CC1(C)c2ccccc2-c2cc(N(c3ccc(-c4ccccc4)cc3)c3ccc(-c4ccc(N(c5ccc(-c6ccccc6)cc5)c5ccc6c(c5)C(C)(C)c5ccccc5-6)cc4-c4ccccc4)cc3)ccc21]

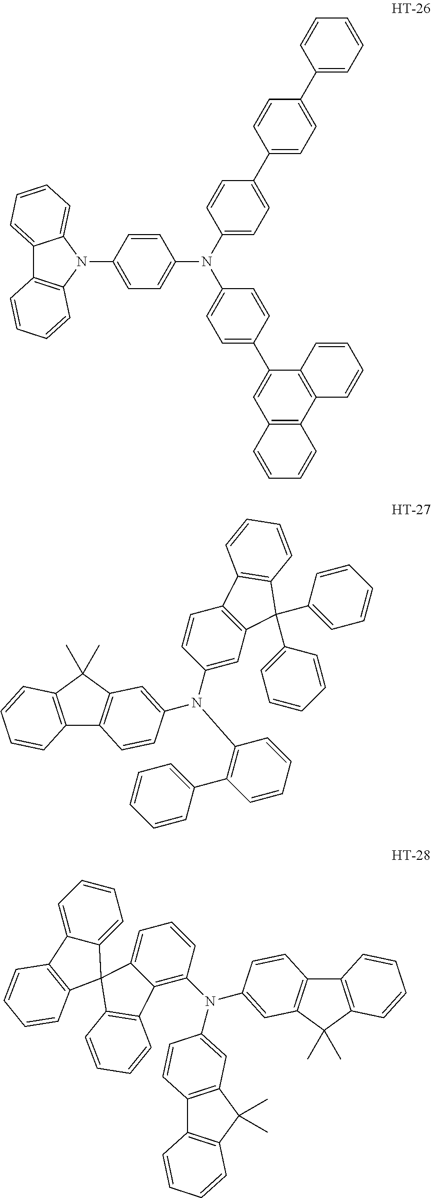

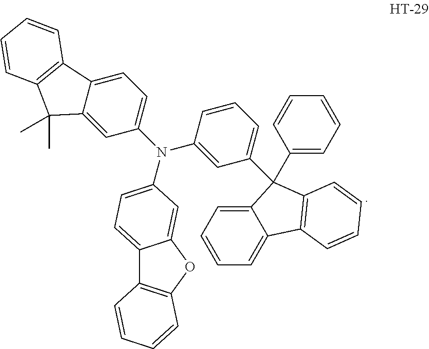 [SMILES: CC1(C)c2ccccc2-c2ccc(N(c3cccc(C4(c5ccccc5)c5ccccc5-c5ccccc54)c3)c3ccc4c(c3)oc3ccccc34)cc21]

In one embodiment, the hole transport layer 320 may consist of HT-27.

Optionally, the electron blocking layer 330 may include one or more hole transport materials, and the hole transport materials may be selected from a carbazole polymer, carbazole connected triarylamine compounds or other types of compounds, which are not specially limited in the present disclosure. For example, in some embodiments of the present disclosure, the electron blocking layer 330 consists of HT-29.

Optionally, the electron blocking layer is also referred to as a hole buffer layer, a hole adjustment layer, a hole auxiliary layer, a light-emitting adjustment layer, a light-emitting auxiliary layer or a second hole transport layer.

Optionally, the hole injection layer 310 may also be arranged between the anode 100 and the hole transport layer 320 to enhance the ability to inject holes into the hole transport layer 320. The hole injection layer 310 may be made of a benzidine derivative, a starburst arylamine compound, a phthalocyanine derivative or other materials, which is not specially limited in the present disclosure. A material of the hole injection layer 310 may be selected from, for example, the following compounds or any combination of them;

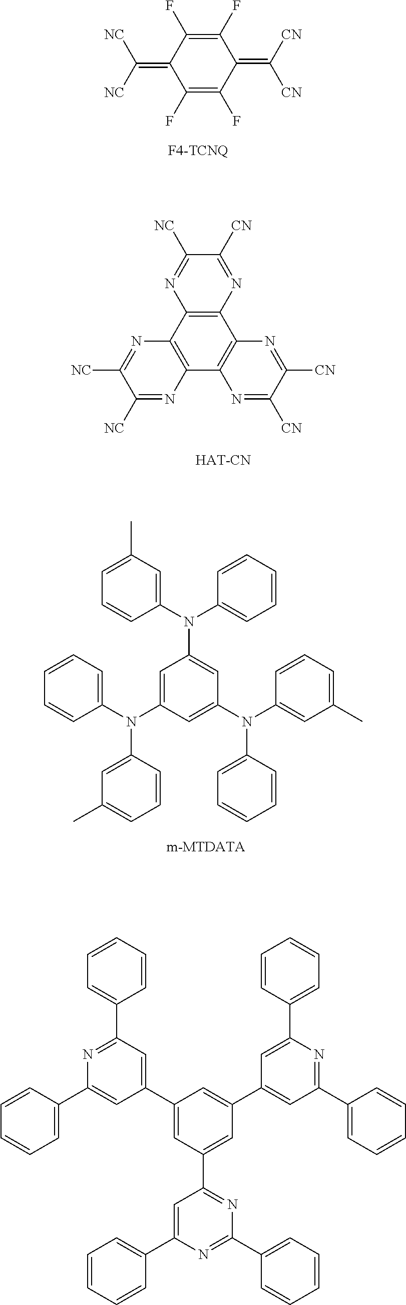 [SMILES: N#CC(C#N)=c1c(F)c(F)c(=C(C#N)C#N)c(F)c1F ; N#Cc1nc2c3nc(C#N)c(C#N)nc3c3nc(C#N)c(C#N)nc3c2nc1C#N ; Cc1cccc(N(c2ccccc2)c2cc(N(c3ccccc3)c3cccc(C)c3)cc(N(c3ccccc3)c3cccc(C)c3)c2)c1 ; c1ccc(-c2cc(-c3cc(-c4cc(-c5ccccc5)nc(-c5ccccc5)c4)cc(-c4cc(-c5ccccc5)nc(-c5ccccc5)n4)c3)cc(-c3ccccc3)n2)cc1]

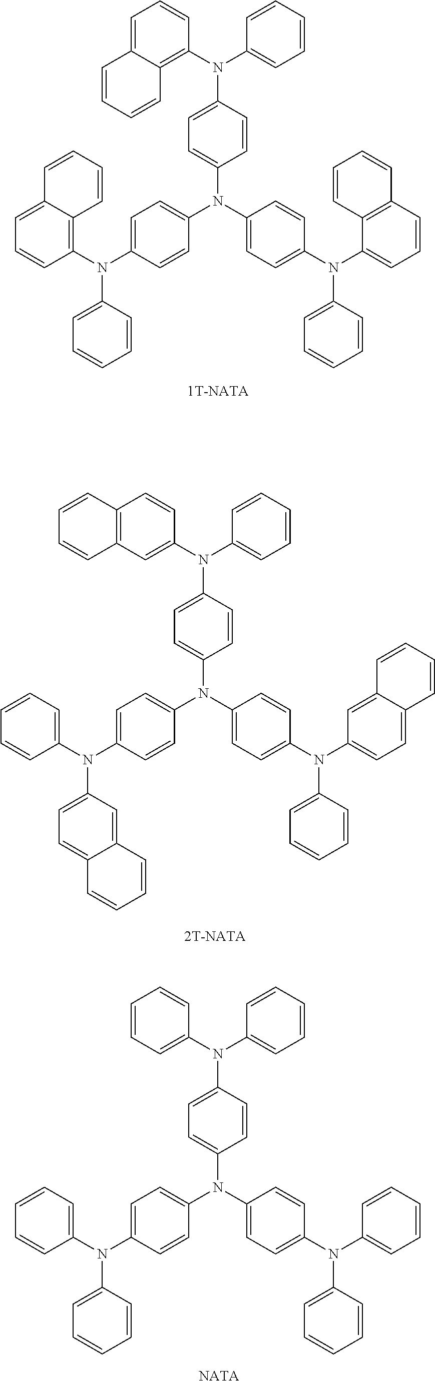

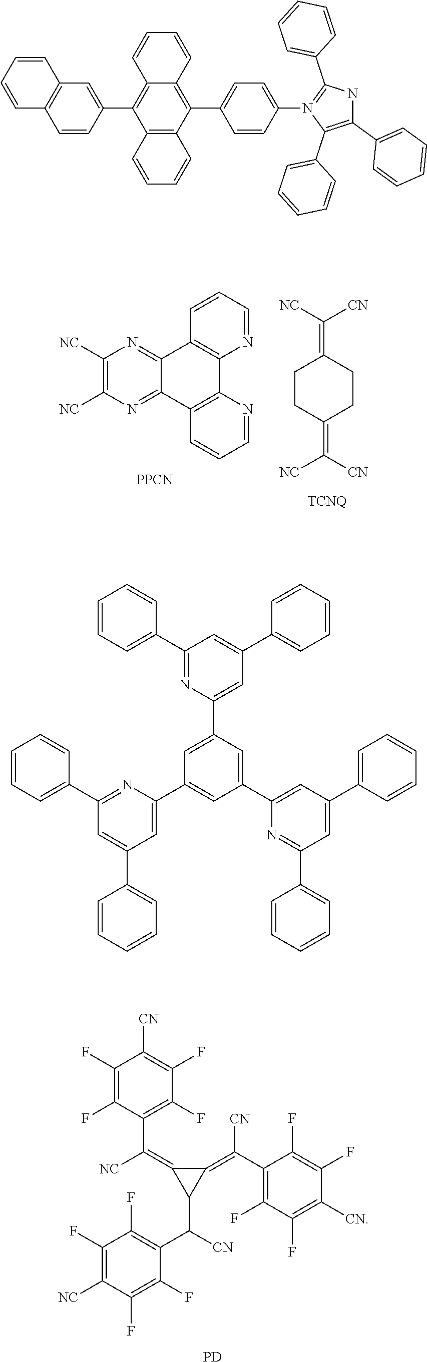

In an embodiment of the present disclosure, the hole injection layer 310 consists of PD and HT-27.

Optionally, the organic light-emitting layer 340 may consist of a single light-emitting material or may include a host material and a guest material. Optionally, the organic light-emitting layer 340 is composed of the host material and the guest material, holes injected into the organic light-emitting layer 340 and electrons injected into the organic light-emitting layer 340 may be recombined in the organic light-emitting layer 340 to form excitons, the excitons transfer energy to the host material, and the host material transfers energy to the guest material, and then the guest material can emit light.

The host material of the organic light-emitting layer 340 may include a metal chelated compound, a bis-styryl derivative, an aromatic amine derivative, a dibenzofuran derivative, or other types of materials. Optionally, the host material includes the nitrogen-containing compound of the present disclosure.

The guest material of the organic light-emitting layer 340 may be a compound having a condensed aryl ring or its derivative, a compound having a heteroaryl ring or its derivative, an aromatic amine derivative, or other materials, which is not particularly limited in the present disclosure. The guest material is also referred to as a doping material or a dopant. The guest material can be divided into fluorescent dopants and phosphorescent dopants according to luminescence types.

In the present disclosure, the guest material for the organic light-emitting layer is a phosphorescent dopant.

In some embodiments of the present disclosure, the guest material used in the organic light-emitting layer of the present disclosure is

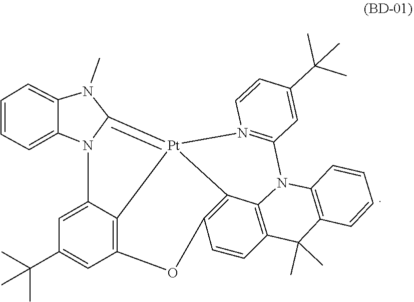

In some embodiments of the present disclosure, the nitrogen-containing compound of the present disclosure is used as a hole-type host material for an organic electroluminescent layer.

In an embodiment of the present disclosure, the organic electroluminescent device is a blue organic electroluminescent device. In a more specific embodiment, the host material of the organic light-emitting layer 340 includes the nitrogen-containing compound of the present disclosure and BH—N, and the guest material is BD-01.

The electron transport layer 350 may be of a single-layer structure or a multi-layer structure, and may include one or more electron transport materials, and the electron transport materials may be selected from, but are not limited to, ET-1, LiQ, a benzimidazole derivative, an oxadiazole derivative, a quinoxaline derivative, or other electron transport materials, which are not particularly limited in the present disclosure. A material of the electron transport layer 350 includes, but is not limited to, the following compounds:

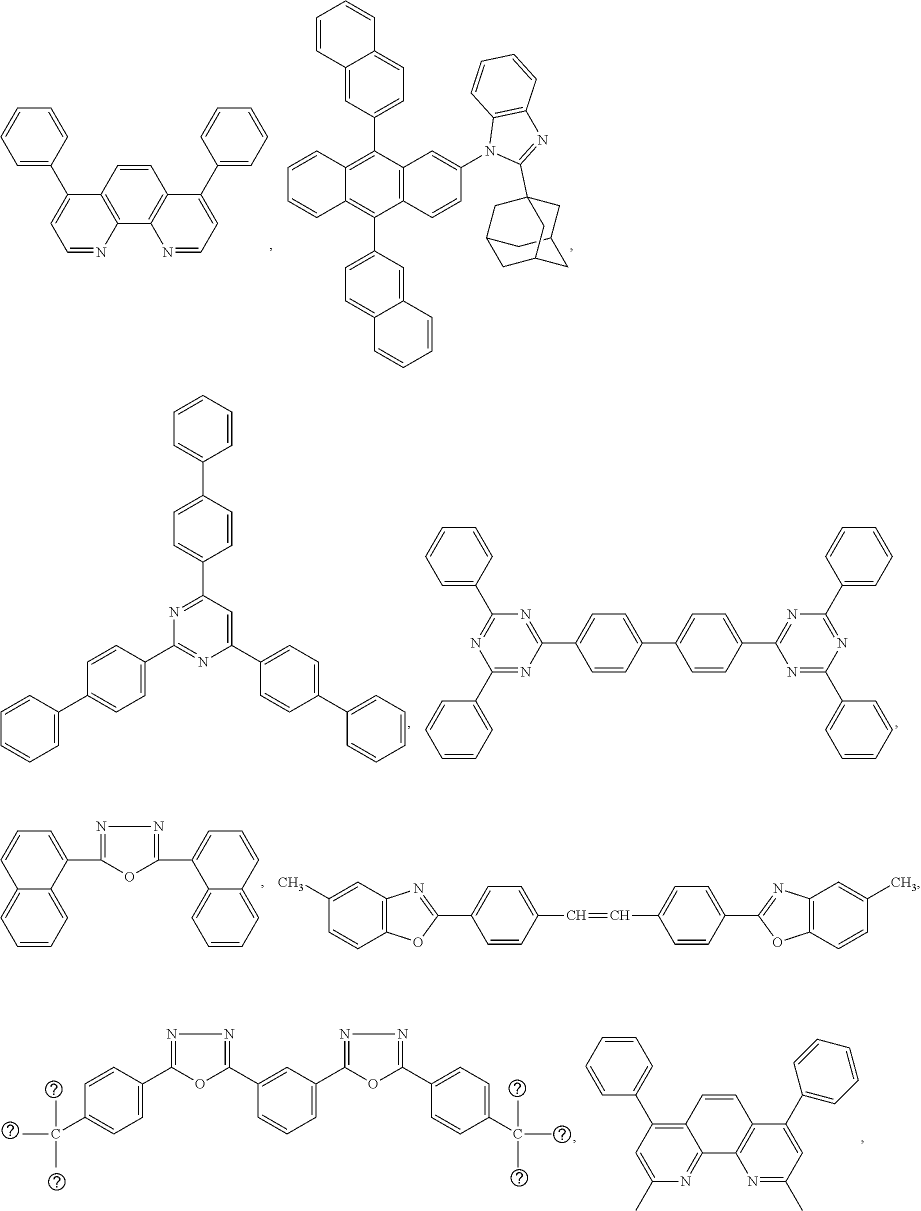

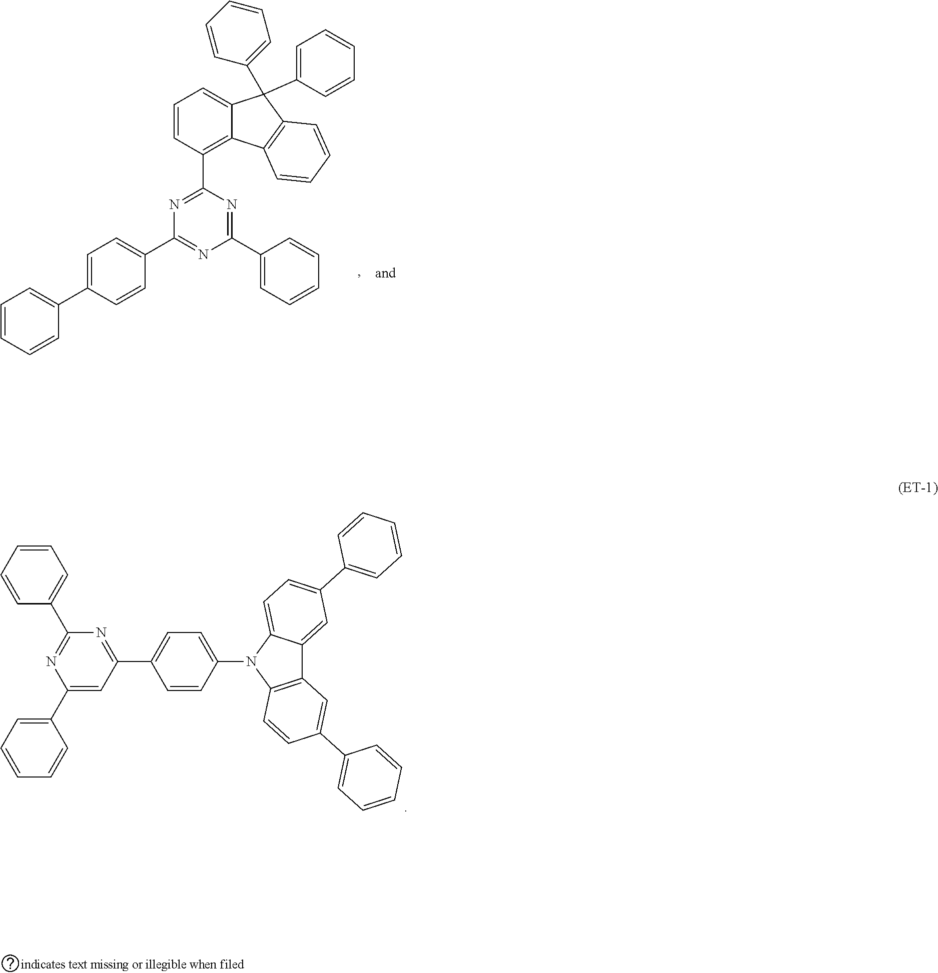 [SMILES: c1ccc(-c2ccc(-c3nc(-c4ccccc4)nc(-c4cccc5c4-c4ccccc4C5(c4ccccc4)c4ccccc4)n3)cc2)cc1 ; c1ccc(-c2ccc3c(c2)c2cc(-c4ccccc4)ccc2n3-c2ccc(-c3cc(-c4ccccc4)nc(-c4ccccc4)n3)cc2)cc1]

In one embodiment of the present disclosure, the electron transport layer 350 may consist of ET-1 and LiQ.

In the present disclosure, the cathode 200 may include a cathode material, which is a material with a small work function that facilitates electron injection into the functional layer. Specific examples of the cathode material include, but are not limited to, metals such as magnesium, calcium, sodium, potassium, titanium, indium, yttrium, lithium, gadolinium, aluminum, silver, tin, and lead, or its alloy; or a plurality of layers of materials such as LiF/Al, Liq/Al, LiO₂/Al, LiF/Ca, LiF/Al, and BaF₂/Ca. Optionally, a metal electrode including magnesium and silver as the cathode is included.

Optionally, the electron injection layer 360 may also be disposed between the cathode 200 and the electron transport layer 350 to enhance the ability to inject electrons into the electron transport layer 350. The electron injection layer 360 may include an inorganic material such as an alkali metal sulfide and an alkali metal halide, or may include a complex of an alkali metal and an organic substance. In one embodiment of the present disclosure, the electron injection layer 360 may include ytterbium (Yb).

A third aspect of the present disclosure provides is an electronic apparatus, including the organic electroluminescent device according to the second aspect of the present disclosure.

According to one embodiment, as shown in FIG. 2, the electronic apparatus provided is an electronic apparatus 400 that includes the organic electroluminescent device described above. The electronic apparatus 400 may be, for example, a display device, a lighting device, an optical communication device, or other type of electronic devices, and may include, for example, but is not limited to, a computer screen, a mobile phone screen, a television, electronic paper, an emergency light, an optical module, and the like.

A synthesis method of the nitrogen-containing compound of the present disclosure is specifically illustrated below with reference to synthesis examples, but the present disclosure is not limited in any way accordingly.

### SYNTHESIS EXAMPLES

Those skilled in the art will recognize that chemical reactions described in the present disclosure may be used to suitably prepare a number of nitrogen-containing compounds of the present disclosure, and that other methods for preparing the compounds of the present disclosure are considered to be within the scope of the present disclosure. For example, the synthesis of those non-exemplified compounds according to the present disclosure can be successfully accomplished by those skilled in the art by modification methods such as appropriately protecting interfering groups, by utilizing other known reagents other than those described in the present disclosure, or by making some conventional modification of reaction conditions. Compounds of which synthesis methods are not mentioned in the present disclosure are all commercially available raw material products.

### Synthesis of Intermediate a1:

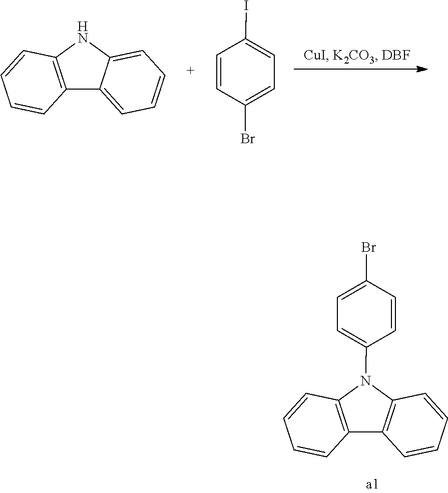 [SMILES: c1ccc2c(c1)[nH]c1ccccc12 ; Brc1ccc(I)cc1 ; Brc1ccc(-n2c3ccccc3c3ccccc32)cc1]

Under nitrogen protection, carbazole (4.0 g; 23.9 mmol), 4-bromoiodobenzene (7.4 g; 26.3 mmol), cuprous iodide (0.9 g; 4.8 mol), anhydrous potassium carbonate (7.3 g; 52.7 mmol), 1,10-phenanthroline (1.7 g; 9.6 mmol), 18-crown-6 (1.3 g; 4.8 mmol) and dimethylformamide (40 mL) were added to a round-bottom flask, heating was performed under stirring to 135° C.-140° C., and a reaction was carried out for 12 h. The reaction was stopped, the reaction solution was cooled to room temperature, deionized water and dichloromethane were added, liquid separation was performed, an organic phase was washed with a large amount of water and dried over anhydrous magnesium sulfate, and a solvent was removed under reduced pressure. The obtained crude product was purified by silica gel column chromatography using a dichloromethane/n-heptane mixed solvent as an eluent to obtain an Intermediate al (5.9 g; yield: 77%) as a white solid.

Referring to the synthesis method of the Intermediate al, intermediates shown in Table 1 below were synthesized by using a Reactant A instead of carbazole and using a Reactant B instead of 4-bromoiodobenzene:

TABLE 1

| Inter- | | | | |
|---|---|---|---|---|
| mediate | | | | Yield |
| No. | Reactant A | Reactant B | Structure | (%) |
| a2 | 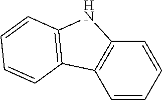 [SMILES: c1ccc2c(c1)[nH]c1ccccc12] | 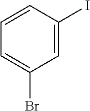 | 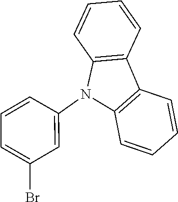 [SMILES: Brc1cccc(-n2c3ccccc3c3ccccc32)c1] | 74 |
| a3 | 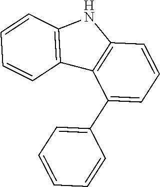 [SMILES: c1ccc(-c2cccc3[nH]c4ccccc4c23)cc1] | 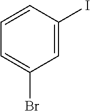 | 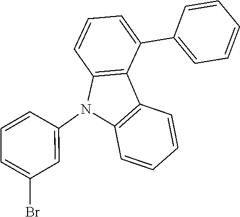 [SMILES: Brc1cccc(-n2c3ccccc3c3c(-c4ccccc4)cccc32)c1] | 84 |
| a4 | 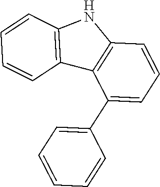 [SMILES: c1ccc(-c2cccc3[nH]c4ccccc4c23)cc1] | 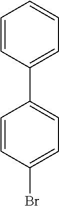 [SMILES: Brc1ccc(-c2ccccc2)cc1] | 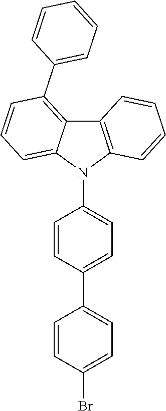 [SMILES: Brc1ccc(-c2ccc(-n3c4ccccc4c4c(-c5ccccc5)cccc43)cc2)cc1] | 74 |
| a5 | 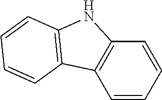 [SMILES: c1ccc2c(c1)[nH]c1ccccc12] | 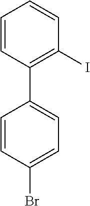 [SMILES: Brc1ccc(-c2ccccc2I)cc1] | 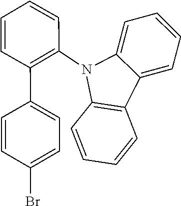 [SMILES: Brc1ccc(-c2ccccc2-n2c3ccccc3c3ccccc32)cc1] | 31 |
| a6 | 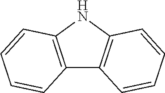 [SMILES: c1ccc2c(c1)[nH]c1ccccc12] | 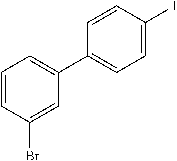 [SMILES: Brc1cccc(-c2ccc(I)cc2)c1] | 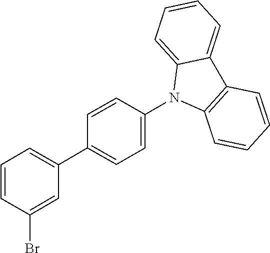 [SMILES: Brc1cccc(-c2ccc(-n3c4ccccc4c4ccccc43)cc2)c1] | 83 |
| a7 | 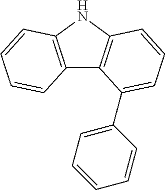 [SMILES: c1ccc(-c2cccc3[nH]c4ccccc4c23)cc1] | 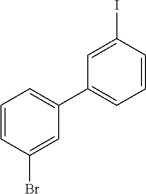 [SMILES: Brc1cccc(-c2cccc(I)c2)c1] | 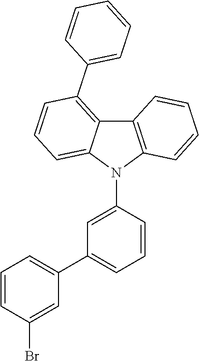 [SMILES: Brc1cccc(-c2cccc(-n3c4ccccc4c4c(-c5ccccc5)cccc43)c2)c1] | 76 |
| a8 | 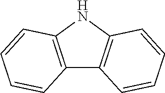 [SMILES: c1ccc2c(c1)[nH]c1ccccc12] | 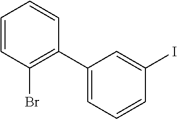 [SMILES: Brc1ccccc1-c1cccc(I)c1] | 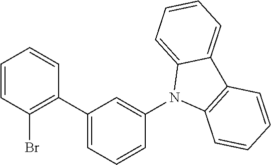 [SMILES: Brc1ccccc1-c1cccc(-n2c3ccccc3c3ccccc32)c1] | 83 |
| a9 | 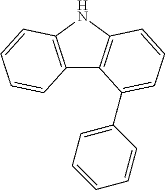 [SMILES: c1ccc(-c2cccc3[nH]c4ccccc4c23)cc1] | 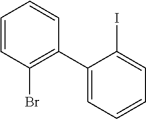 [SMILES: Brc1ccccc1-c1ccccc1I] | 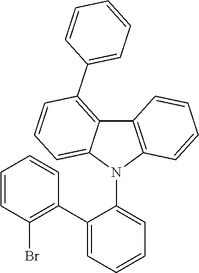 [SMILES: Brc1ccccc1-c1ccccc1-n1c2ccccc2c2c(-c3ccccc3)cccc21] | 39 |
| a10 | 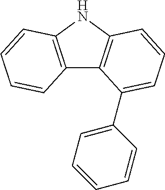 [SMILES: c1ccc(-c2cccc3[nH]c4ccccc4c23)cc1] | 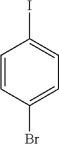 | 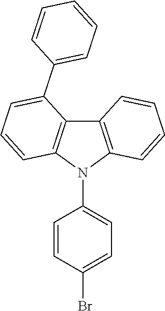 [SMILES: Brc1ccc(-n2c3ccccc3c3c(-c4ccccc4)cccc32)cc1] | 76 |
| a11 | 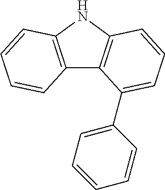 [SMILES: c1ccc(-c2cccc3[nH]c4ccccc4c23)cc1] | 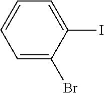 | 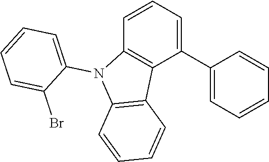 [SMILES: Brc1ccccc1-n1c2ccccc2c2c(-c3ccccc3)cccc21] | 63 |
| a12 | 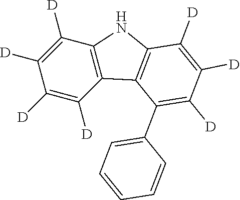 [SMILES: [2H]c1c([2H])c([2H])c2c([nH]c3c([2H])c([2H])c([2H])c(-c4ccccc4)c32)c1[2H]] | 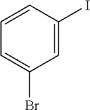 | 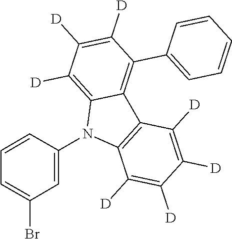 [SMILES: [2H]c1c([2H])c([2H])c2c(c1[2H])c1c(-c3ccccc3)c([2H])c([2H])c([2H])c1n2-c1cccc(Br)c1] | 84 |
| a13 | 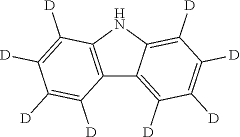 [SMILES: [2H]c1c([2H])c([2H])c2c([nH]c3c([2H])c([2H])c([2H])c([2H])c32)c1[2H]] | 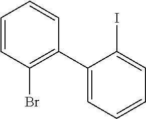 [SMILES: Brc1ccccc1-c1ccccc1I] | 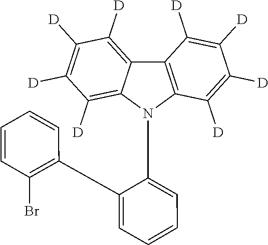 [SMILES: [2H]c1c([2H])c([2H])c2c(c1[2H])c1c([2H])c([2H])c([2H])c([2H])c1n2-c1ccccc1-c1ccccc1Br] | 44 |
| a14 | 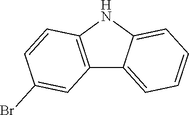 [SMILES: Brc1ccc2[nH]c3ccccc3c2c1] | 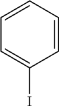 | 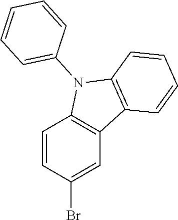 [SMILES: Brc1ccc2c(c1)c1ccccc1n2-c1ccccc1] | 82 |
| a15 | 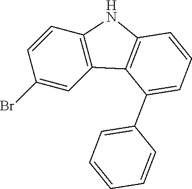 [SMILES: Brc1ccc2[nH]c3cccc(-c4ccccc4)c3c2c1] | 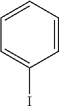 | 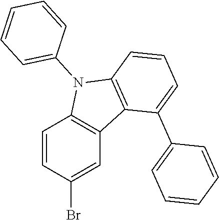 [SMILES: Brc1ccc2c(c1)c1c(-c3ccccc3)cccc1n2-c1ccccc1] | 77 |
| a16 | 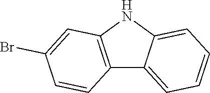 [SMILES: Brc1ccc2c(c1)[nH]c1ccccc12] | 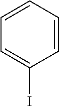 | 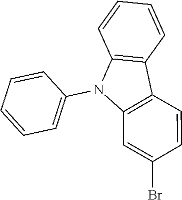 [SMILES: Brc1ccc2c3ccccc3n(-c3ccccc3)c2c1] | 68 |
| a17 | 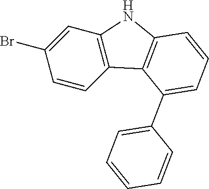 [SMILES: Brc1ccc2c(c1)[nH]c1cccc(-c3ccccc3)c12] | 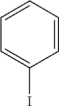 | 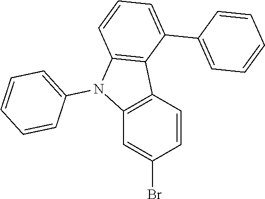 [SMILES: Brc1ccc2c3c(-c4ccccc4)cccc3n(-c3ccccc3)c2c1] | 73 |
| a18 | 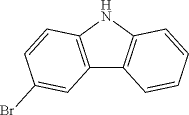 [SMILES: Brc1ccc2[nH]c3ccccc3c2c1] | 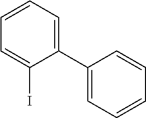 [SMILES: Ic1ccccc1-c1ccccc1] | 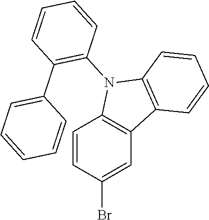 [SMILES: Brc1ccc2c(c1)c1ccccc1n2-c1ccccc1-c1ccccc1] | 39 |
| a19 | 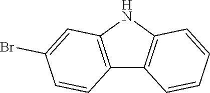 [SMILES: Brc1ccc2c(c1)[nH]c1ccccc12] | 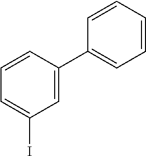 [SMILES: Ic1cccc(-c2ccccc2)c1] | 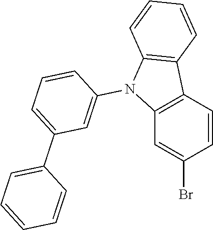 [SMILES: Brc1ccc2c3ccccc3n(-c3cccc(-c4ccccc4)c3)c2c1] | 80 |
| a20 | 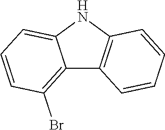 [SMILES: Brc1cccc2[nH]c3ccccc3c12] | 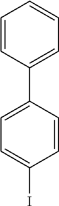 [SMILES: Ic1ccc(-c2ccccc2)cc1] | 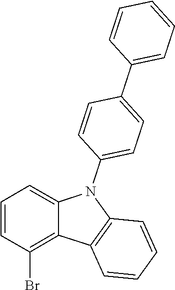 [SMILES: Brc1cccc2c1c1ccccc1n2-c1ccc(-c2ccccc2)cc1] | 81 |
| a21 | 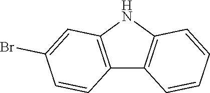 [SMILES: Brc1ccc2c(c1)[nH]c1ccccc12] | 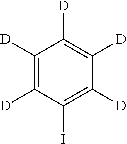 [SMILES: [2H]c1c([2H])c([2H])c(I)c([2H])c1[2H]] | 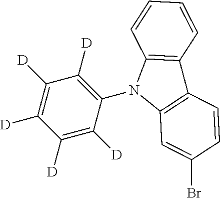 [SMILES: [2H]c1c([2H])c([2H])c(-n2c3ccccc3c3ccc(Br)cc32)c([2H])c1[2H]] | 82 |
| a22 | 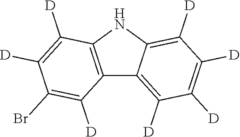 [SMILES: [2H]c1c([2H])c([2H])c2c([nH]c3c([2H])c([2H])c(Br)c([2H])c32)c1[2H]] | 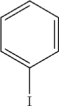 | 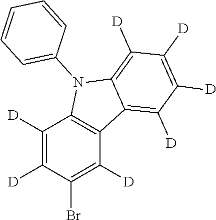 [SMILES: [2H]c1c([2H])c([2H])c2c(c1[2H])c1c([2H])c(Br)c([2H])c([2H])c1n2-c1ccccc1] | 84 |
| a23 | 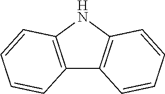 [SMILES: c1ccc2c(c1)[nH]c1ccccc12] | 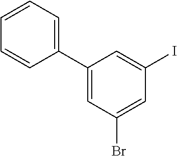 [SMILES: Brc1cc(I)cc(-c2ccccc2)c1] | 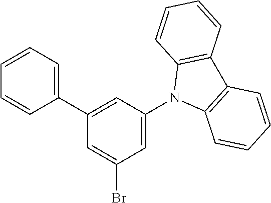 [SMILES: Brc1cc(-c2ccccc2)cc(-n2c3ccccc3c3ccccc32)c1] | 75 |
| a24 | 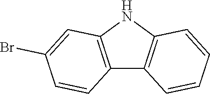 [SMILES: Brc1ccc2c(c1)[nH]c1ccccc12] | 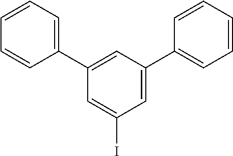 [SMILES: Ic1cc(-c2ccccc2)cc(-c2ccccc2)c1] | 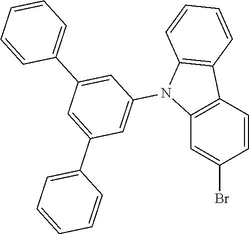 [SMILES: Brc1ccc2c3ccccc3n(-c3cc(-c4ccccc4)cc(-c4ccccc4)c3)c2c1] | 67 |

### Synthesis of Intermediate b1:

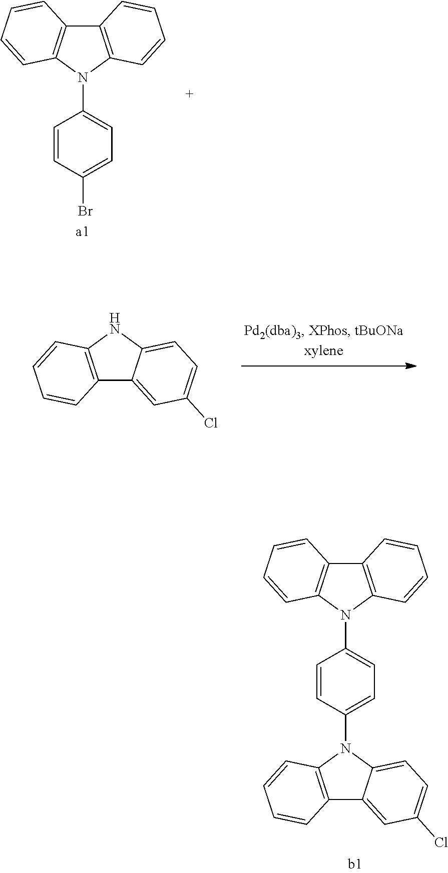 [SMILES: Brc1ccc(-n2c3ccccc3c3ccccc32)cc1 ; Clc1ccc2[nH]c3ccccc3c2c1 ; Clc1ccc2c(c1)c1ccccc1n2-c1ccc(-n2c3ccccc3c3ccccc32)cc1]

Under nitrogen protection, the Intermediate al (5.7 g; 17.8 mmol), 3-chlorocarbazole (3.4 g; 16.9 mol), tris(dibenzylideneacetone) dipalladium (0.2 g; 0.2 mmol), 2-dicyclohexylphosphino-2′,4′,6′-triisopropylbiphenyl (0.2 g; 0.4 mmol), sodium tert-butoxide (2.6 g; 26.6 mmol) and xylene (60 mL) were added to a round-bottom flask, and a reaction was carried out under stirring at 135° C.-140° C. for 8 h. The resulting reaction solution was cooled to room temperature, the reaction solution was washed with water, and then liquid separation was performed, an organic phase was dried over anhydrous magnesium sulfate, and a solvent was removed under reduced pressure to obtain a crude product. The crude product was purified by silica gel column chromatography using a dichloromethane/n-heptane mixed solvent as an eluent, and the obtained sample was purified by recrystallization using a dichloromethane/n-heptane mixed solvent to obtain an Intermediate b1 (5.2 g; yield: 70%) as a white solid.

Referring to the synthesis method of the Intermediate b1, intermediates shown in Table 2 below were synthesized by using a Reactant C instead of the Intermediate al and using a Reactant D instead of 3-chlorocarbazole:

TABLE 2

| Intermediate | | | | Yield |
|---|---|---|---|---|
| No. | Reactant C | Reactant D | Structure | (%) |
| b2 | 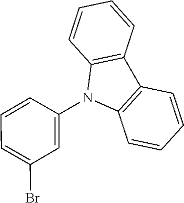 [SMILES: Brc1cccc(-n2c3ccccc3c3ccccc32)c1] | 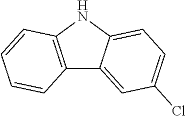 [SMILES: Clc1ccc2[nH]c3ccccc3c2c1] | 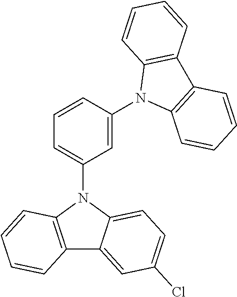 [SMILES: Clc1ccc2c(c1)c1ccccc1n2-c1cccc(-n2c3ccccc3c3ccccc32)c1] | 67 |
| b3 | 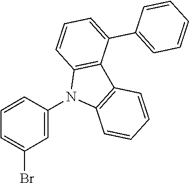 [SMILES: Brc1cccc(-n2c3ccccc3c3c(-c4ccccc4)cccc32)c1] | 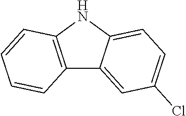 [SMILES: Clc1ccc2[nH]c3ccccc3c2c1] | 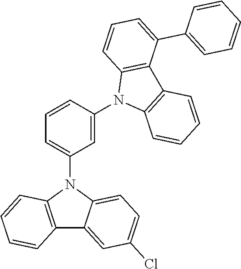 [SMILES: Clc1ccc2c(c1)c1ccccc1n2-c1cccc(-n2c3ccccc3c3c(-c4ccccc4)cccc32)c1] | 77 |
| b4 | 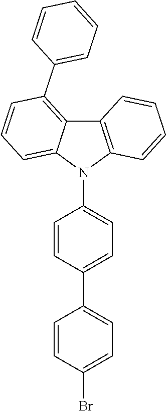 [SMILES: Brc1ccc(-c2ccc(-n3c4ccccc4c4c(-c5ccccc5)cccc43)cc2)cc1] | 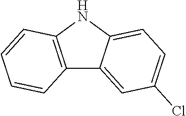 [SMILES: Clc1ccc2[nH]c3ccccc3c2c1] | 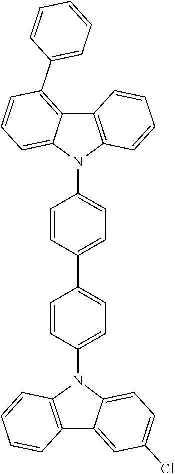 [SMILES: Clc1ccc2c(c1)c1ccccc1n2-c1ccc(-c2ccc(-n3c4ccccc4c4c(-c5ccccc5)cccc43)cc2)cc1] | 71 |
| b5 | 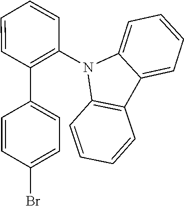 [SMILES: Brc1ccc(-c2ccccc2-n2c3ccccc3c3ccccc32)cc1] | 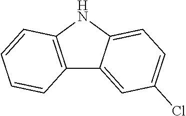 [SMILES: Clc1ccc2[nH]c3ccccc3c2c1] | 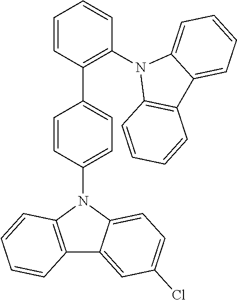 [SMILES: Clc1ccc2c(c1)c1ccccc1n2-c1ccc(-c2ccccc2-n2c3ccccc3c3ccccc32)cc1] | 67 |
| b6 | 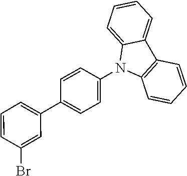 [SMILES: Brc1cccc(-c2ccc(-n3c4ccccc4c4ccccc43)cc2)c1] | 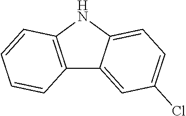 [SMILES: Clc1ccc2[nH]c3ccccc3c2c1] | 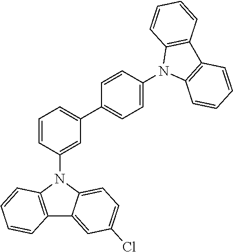 [SMILES: Clc1ccc2c(c1)c1ccccc1n2-c1cccc(-c2ccc(-n3c4ccccc4c4ccccc43)cc2)c1] | 78 |
| b7 | 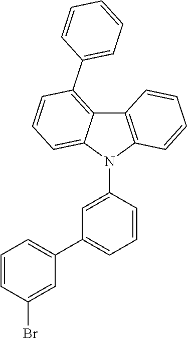 [SMILES: Brc1cccc(-c2cccc(-n3c4ccccc4c4c(-c5ccccc5)cccc43)c2)c1] | 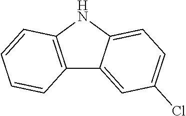 [SMILES: Clc1ccc2[nH]c3ccccc3c2c1] | 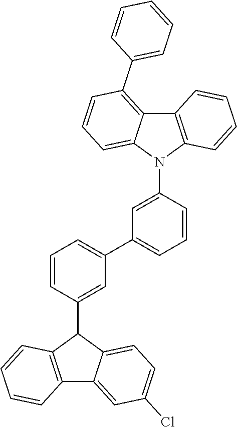 [SMILES: Clc1ccc2c(c1)-c1ccccc1C2c1cccc(-c2cccc(-n3c4ccccc4c4c(-c5ccccc5)cccc43)c2)c1] | 71 |
| b8 | 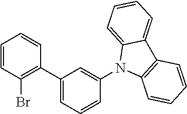 [SMILES: Brc1ccccc1-c1cccc(-n2c3ccccc3c3ccccc32)c1] | 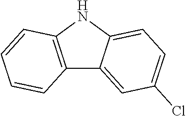 [SMILES: Clc1ccc2[nH]c3ccccc3c2c1] | 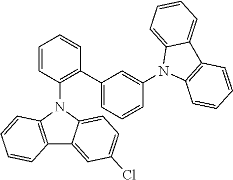 [SMILES: Clc1ccc2c(c1)c1ccccc1n2-c1ccccc1-c1cccc(-n2c3ccccc3c3ccccc32)c1] | 39 |
| b9 | 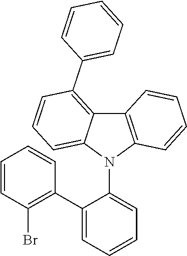 [SMILES: Brc1ccccc1-c1ccccc1-n1c2ccccc2c2c(-c3ccccc3)cccc21] | 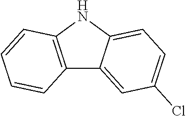 [SMILES: Clc1ccc2[nH]c3ccccc3c2c1] | 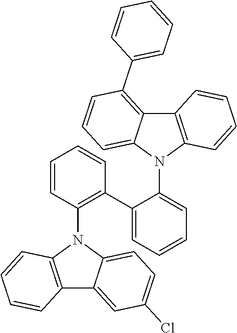 [SMILES: Clc1ccc2c(c1)c1ccccc1n2-c1ccccc1-c1ccccc1-n1c2ccccc2c2c(-c3ccccc3)cccc21] | 30 |
| b10 | 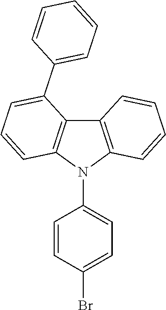 [SMILES: Brc1ccc(-n2c3ccccc3c3c(-c4ccccc4)cccc32)cc1] | 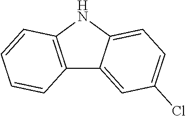 [SMILES: Clc1ccc2[nH]c3ccccc3c2c1] | 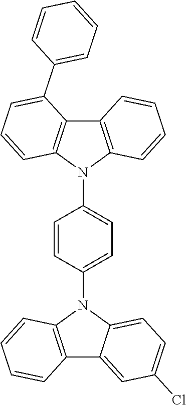 [SMILES: Clc1ccc2c(c1)c1ccccc1n2-c1ccc(-n2c3ccccc3c3c(-c4ccccc4)cccc32)cc1] | 76 |
| b11 | 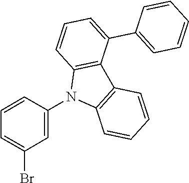 [SMILES: Brc1cccc(-n2c3ccccc3c3c(-c4ccccc4)cccc32)c1] | 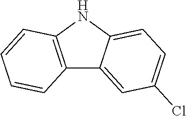 [SMILES: Clc1ccc2[nH]c3ccccc3c2c1] | 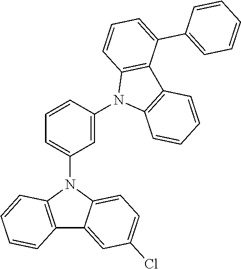 [SMILES: Clc1ccc2c(c1)c1ccccc1n2-c1cccc(-n2c3ccccc3c3c(-c4ccccc4)cccc32)c1] | 70 |
| b12 | 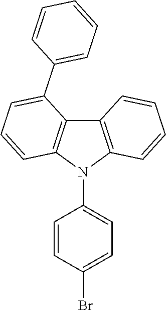 [SMILES: Brc1ccc(-n2c3ccccc3c3c(-c4ccccc4)cccc32)cc1] | 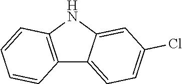 [SMILES: Clc1ccc2c(c1)[nH]c1ccccc12] | 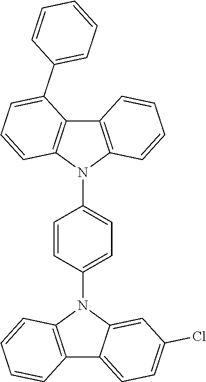 [SMILES: Clc1ccc2c3ccccc3n(-c3ccc(-n4c5ccccc5c5c(-c6ccccc6)cccc54)cc3)c2c1] | 76 |
| b13 | 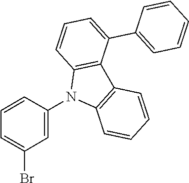 [SMILES: Brc1cccc(-n2c3ccccc3c3c(-c4ccccc4)cccc32)c1] | 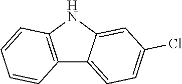 [SMILES: Clc1ccc2c(c1)[nH]c1ccccc12] | 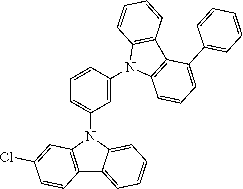 [SMILES: Clc1ccc2c3ccccc3n(-c3cccc(-n4c5ccccc5c5c(-c6ccccc6)cccc54)c3)c2c1] | 71 |
| b14 | 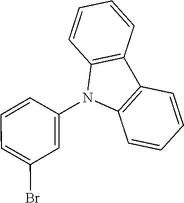 [SMILES: Brc1cccc(-n2c3ccccc3c3ccccc32)c1] | 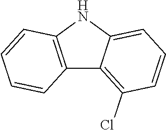 [SMILES: Clc1cccc2[nH]c3ccccc3c12] | 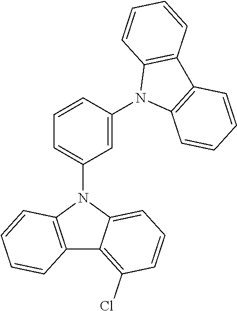 [SMILES: Clc1cccc2c1c1ccccc1n2-c1cccc(-n2c3ccccc3c3ccccc32)c1] | 63 |
| b15 | 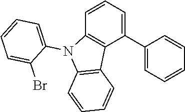 [SMILES: Brc1ccccc1-n1c2ccccc2c2c(-c3ccccc3)cccc21] | 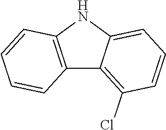 [SMILES: Clc1cccc2[nH]c3ccccc3c12] | 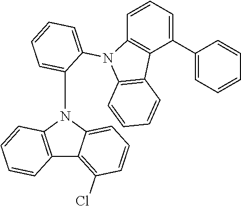 [SMILES: Clc1cccc2c1c1ccccc1n2-c1ccccc1-n1c2ccccc2c2c(-c3ccccc3)cccc21] | 42 |
| b16 | 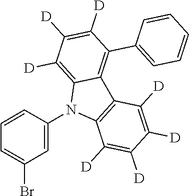 [SMILES: [2H]c1c([2H])c([2H])c2c(c1[2H])c1c(-c3ccccc3)c([2H])c([2H])c([2H])c1n2-c1cccc(Br)c1] | 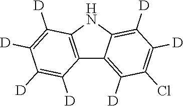 [SMILES: [2H]c1c([2H])c([2H])c2c([nH]c3c([2H])c([2H])c(Cl)c([2H])c32)c1[2H]] | 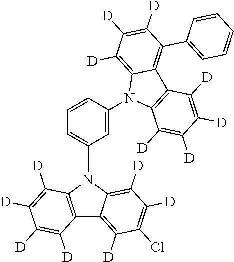 [SMILES: [2H]c1c([2H])c([2H])c2c(c1[2H])c1c([2H])c(Cl)c([2H])c([2H])c1n2-c1cccc(-n2c3c([2H])c([2H])c([2H])c([2H])c3c3c(-c4ccccc4)c([2H])c([2H])c([2H])c32)c1] | 64 |
| b17 | 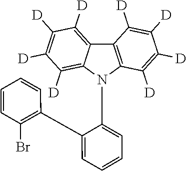 [SMILES: [2H]c1c([2H])c([2H])c2c(c1[2H])c1c([2H])c([2H])c([2H])c([2H])c1n2-c1ccccc1-c1ccccc1Br] | 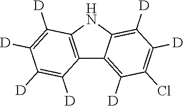 [SMILES: [2H]c1c([2H])c([2H])c2c([nH]c3c([2H])c([2H])c(Cl)c([2H])c32)c1[2H]] | 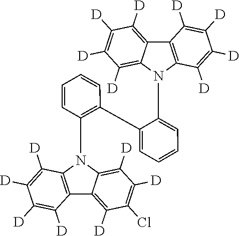 [SMILES: [2H]c1c([2H])c([2H])c2c(c1[2H])c1c([2H])c([2H])c([2H])c([2H])c1n2-c1ccccc1-c1ccccc1-n1c2c([2H])c([2H])c([2H])c([2H])c2c2c([2H])c(Cl)c([2H])c([2H])c21] | 37 |
| b18 | 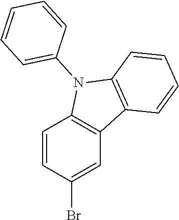 [SMILES: Brc1ccc2c(c1)c1ccccc1n2-c1ccccc1] | 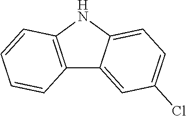 [SMILES: Clc1ccc2[nH]c3ccccc3c2c1] | 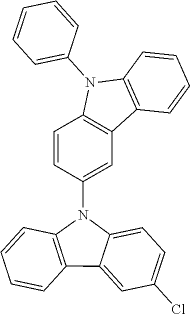 [SMILES: Clc1ccc2c(c1)c1ccccc1n2-c1ccc2c(c1)c1ccccc1n2-c1ccccc1] | 66 |
| b19 | 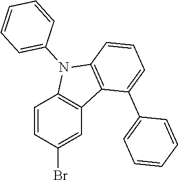 [SMILES: Brc1ccc2c(c1)c1c(-c3ccccc3)cccc1n2-c1ccccc1] | 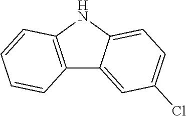 [SMILES: Clc1ccc2[nH]c3ccccc3c2c1] | 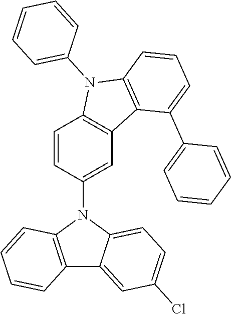 [SMILES: Clc1ccc2c(c1)c1ccccc1n2-c1ccc2c(c1)c1c(-c3ccccc3)cccc1n2-c1ccccc1] | 52 |
| b20 | 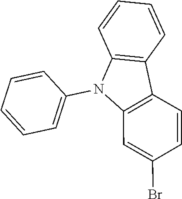 [SMILES: Brc1ccc2c3ccccc3n(-c3ccccc3)c2c1] | 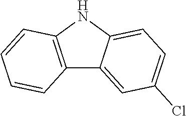 [SMILES: Clc1ccc2[nH]c3ccccc3c2c1] | 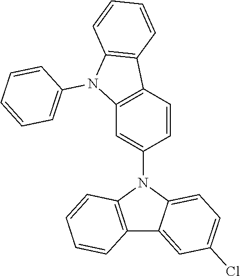 [SMILES: Clc1ccc2c(c1)c1ccccc1n2-c1ccc2c3ccccc3n(-c3ccccc3)c2c1] | 63 |
| b21 | 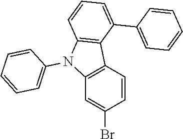 [SMILES: Brc1ccc2c3c(-c4ccccc4)cccc3n(-c3ccccc3)c2c1] | 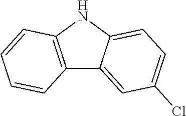 [SMILES: Clc1ccc2[nH]c3ccccc3c2c1] | 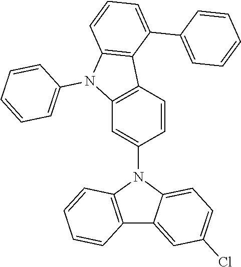 [SMILES: Clc1ccc2c(c1)c1ccccc1n2-c1ccc2c3c(-c4ccccc4)cccc3n(-c3ccccc3)c2c1] | 76 |
| b22 | 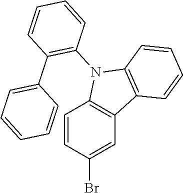 [SMILES: Brc1ccc2c(c1)c1ccccc1n2-c1ccccc1-c1ccccc1] | 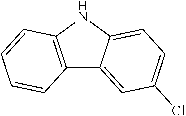 [SMILES: Clc1ccc2[nH]c3ccccc3c2c1] | 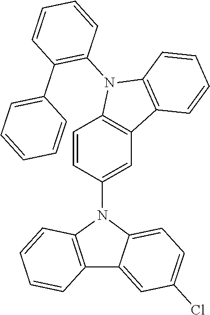 [SMILES: Clc1ccc2c(c1)c1ccccc1n2-c1ccc2c(c1)c1ccccc1n2-c1ccccc1-c1ccccc1] | 69 |
| b23 | 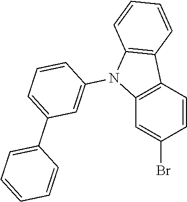 [SMILES: Brc1ccc2c3ccccc3n(-c3cccc(-c4ccccc4)c3)c2c1] | 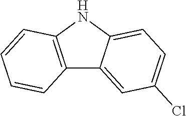 [SMILES: Clc1ccc2[nH]c3ccccc3c2c1] | 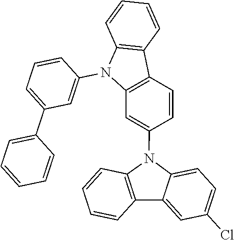 [SMILES: Clc1ccc2c(c1)c1ccccc1n2-c1ccc2c3ccccc3n(-c3cccc(-c4ccccc4)c3)c2c1] | 75 |
| b24 | 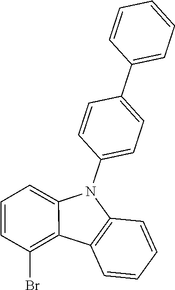 [SMILES: Brc1cccc2c1c1ccccc1n2-c1ccc(-c2ccccc2)cc1] | 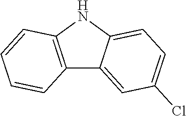 [SMILES: Clc1ccc2[nH]c3ccccc3c2c1] | 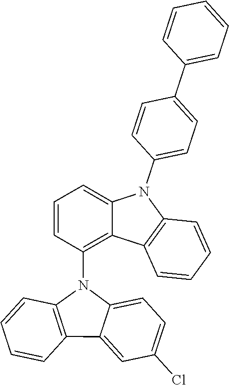 [SMILES: Clc1ccc2c(c1)c1ccccc1n2-c1cccc2c1c1ccccc1n2-c1ccc(-c2ccccc2)cc1] | 41 |
| b25 | 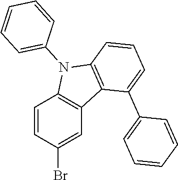 [SMILES: Brc1ccc2c(c1)c1c(-c3ccccc3)cccc1n2-c1ccccc1] | 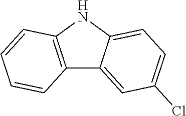 [SMILES: Clc1ccc2[nH]c3ccccc3c2c1] | 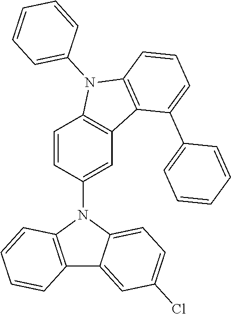 [SMILES: Clc1ccc2c(c1)c1ccccc1n2-c1ccc2c(c1)c1c(-c3ccccc3)cccc1n2-c1ccccc1] | 56 |
| b26 | 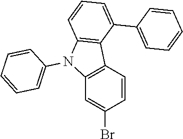 [SMILES: Brc1ccc2c3c(-c4ccccc4)cccc3n(-c3ccccc3)c2c1] | 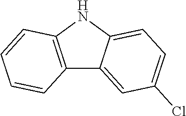 [SMILES: Clc1ccc2[nH]c3ccccc3c2c1] | 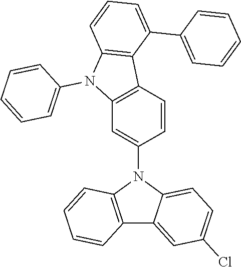 [SMILES: Clc1ccc2c(c1)c1ccccc1n2-c1ccc2c3c(-c4ccccc4)cccc3n(-c3ccccc3)c2c1] | 71 |
| b27 | 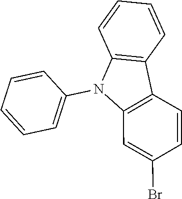 [SMILES: Brc1ccc2c3ccccc3n(-c3ccccc3)c2c1] | 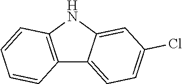 [SMILES: Clc1ccc2c(c1)[nH]c1ccccc12] | 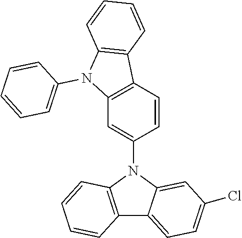 [SMILES: Clc1ccc2c3ccccc3n(-c3ccc4c5ccccc5n(-c5ccccc5)c4c3)c2c1] | 80 |
| b28 | 195 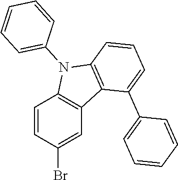 [SMILES: Brc1ccc2c(c1)c1c(-c3ccccc3)cccc1n2-c1ccccc1] | 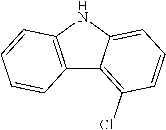 [SMILES: Clc1cccc2[nH]c3ccccc3c12] | 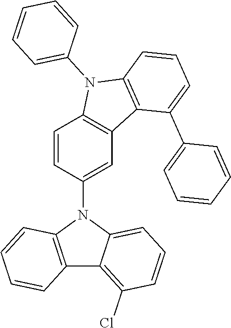 [SMILES: Clc1cccc2c1c1ccccc1n2-c1ccc2c(c1)c1c(-c3ccccc3)cccc1n2-c1ccccc1] | 58 |
| b29 | 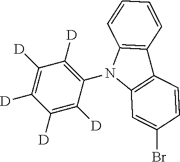 [SMILES: [2H]c1c([2H])c([2H])c(-n2c3ccccc3c3ccc(Br)cc32)c([2H])c1[2H]] | 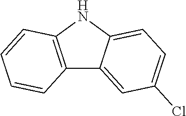 [SMILES: Clc1ccc2[nH]c3ccccc3c2c1] | 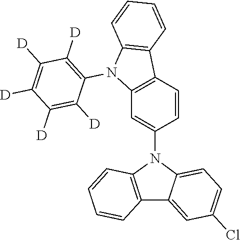 [SMILES: [2H]c1c([2H])c([2H])c(-n2c3ccccc3c3ccc(-n4c5ccccc5c5cc(Cl)ccc54)cc32)c([2H])c1[2H]] | 68 |
| b30 | 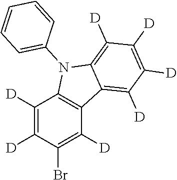 [SMILES: [2H]c1c([2H])c([2H])c2c(c1[2H])c1c([2H])c(Br)c([2H])c([2H])c1n2-c1ccccc1] | 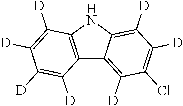 [SMILES: [2H]c1c([2H])c([2H])c2c([nH]c3c([2H])c([2H])c(Cl)c([2H])c32)c1[2H]] | 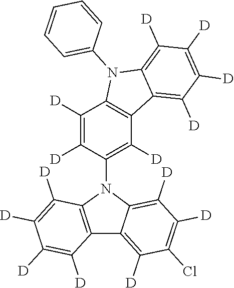 [SMILES: [2H]c1c([2H])c([2H])c2c(c1[2H])c1c([2H])c(-n3c4c([2H])c([2H])c([2H])c([2H])c4c4c([2H])c(Cl)c([2H])c([2H])c43)c([2H])c([2H])c1n2-c1ccccc1] | 80 |
| b31 | 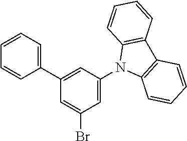 [SMILES: Brc1cc(-c2ccccc2)cc(-n2c3ccccc3c3ccccc32)c1] | 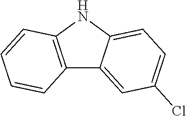 [SMILES: Clc1ccc2[nH]c3ccccc3c2c1] | 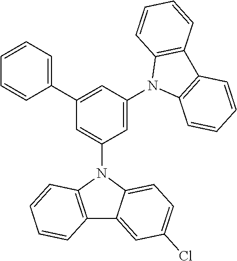 [SMILES: Clc1ccc2c(c1)c1ccccc1n2-c1cc(-c2ccccc2)cc(-n2c3ccccc3c3ccccc32)c1] | 65 |
| b32 | 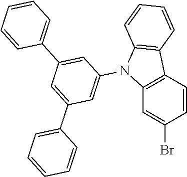 [SMILES: Brc1ccc2c3ccccc3n(-c3cc(-c4ccccc4)cc(-c4ccccc4)c3)c2c1] | 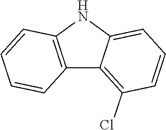 [SMILES: Clc1cccc2[nH]c3ccccc3c12] | 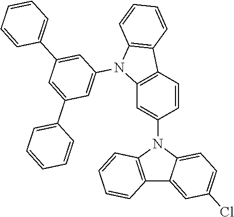 [SMILES: Clc1ccc2c(c1)c1ccccc1n2-c1ccc2c3ccccc3n(-c3cc(-c4ccccc4)cc(-c4ccccc4)c3)c2c1] | 70 |

### Synthesis of Compound A1:

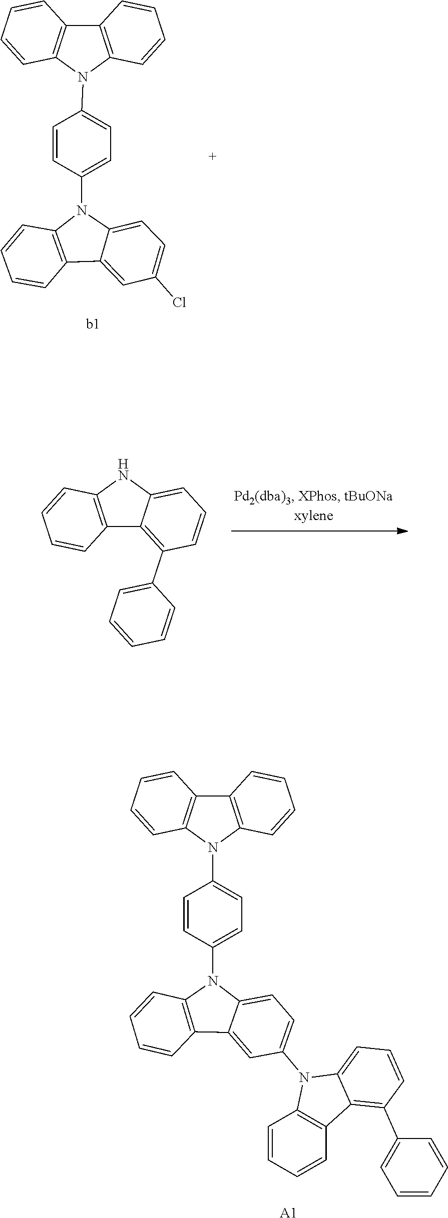

Under nitrogen protection, the Intermediate b1 (5.1 g; 11.5 mmol), 4-phenylcarbazole (2.9 g; 11.8 mmol), tris(dibenzylideneacetone) dipalladium (0.1 g; 0.1 mmol), 2-dicyclohexylphosphino-2′,4′,6′-triisopropylbiphenyl (0.1 g; 0.2 mmol), sodium tert-butoxide (1.7 g; 17.3 mmol) and xylene (50 mL) were added to a round-bottom flask, and a reaction was carried out under stirring at 135° C. to 140° C. for 24 h. The resulting reaction solution was cooled to room temperature, the reaction solution was washed with water, and then liquid separation was performed, an organic phase was dried over anhydrous magnesium sulfate, and a solvent was removed under reduced pressure to obtain a crude product. The crude product was purified by silica gel column chromatography using a mixed solvent of toluene/n-heptane as an eluent, and the obtained sample was purified by recrystallization using a mixed solvent of toluene/n-heptane to obtain a Compound A1 (5.3 g; yield: 71%) as a white solid.

Referring to the synthesis method of the Compound A1, compounds shown in Table 3 below were synthesized by using a Reactant E instead of the Intermediate b1, and using a Reactant F instead of 4-phenylcarbazole:

TABLE 3

| Compound | | | | |
|---|---|---|---|---|
| No. | Reactant E | Reactant F | Structure | Yield (%) |
| A3 | 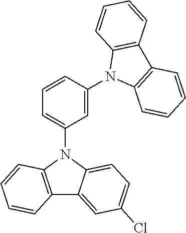 [SMILES: Clc1ccc2c(c1)c1ccccc1n2-c1cccc(-n2c3ccccc3c3ccccc32)c1] | 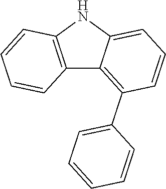 [SMILES: c1ccc(-c2cccc3[nH]c4ccccc4c23)cc1] | 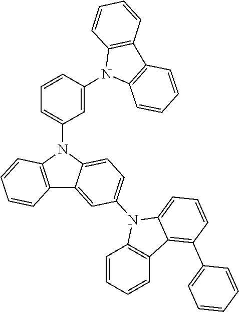 [SMILES: c1ccc(-c2cccc3c2c2ccccc2n3-c2ccc3c(c2)c2ccccc2n3-c2cccc(-n3c4ccccc4c4ccccc43)c2)cc1] | 76 |
| A4 | 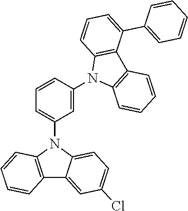 [SMILES: Clc1ccc2c(c1)c1ccccc1n2-c1cccc(-n2c3ccccc3c3c(-c4ccccc4)cccc32)c1] | 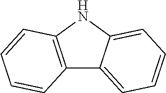 [SMILES: c1ccc2c(c1)[nH]c1ccccc12] | 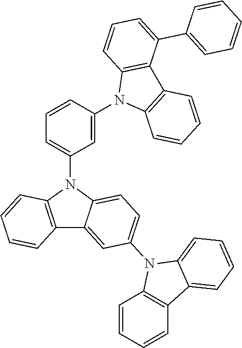 [SMILES: c1ccc(-c2cccc3c2c2ccccc2n3-c2cccc(-n3c4ccccc4c4cc(-n5c6ccccc6c6ccccc65)ccc43)c2)cc1] | 63 |
| A8 | 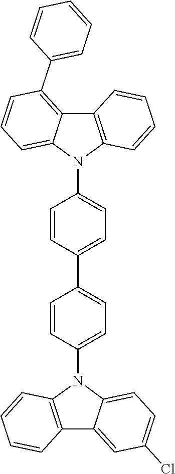 [SMILES: Clc1ccc2c(c1)c1ccccc1n2-c1ccc(-c2ccc(-n3c4ccccc4c4c(-c5ccccc5)cccc43)cc2)cc1] | 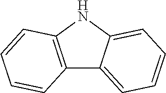 [SMILES: c1ccc2c(c1)[nH]c1ccccc12] | 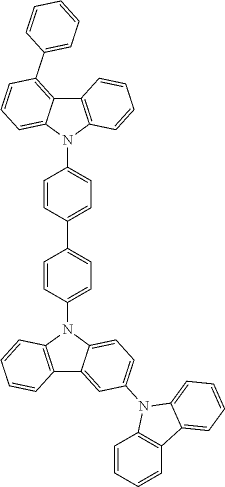 [SMILES: c1ccc(-c2cccc3c2c2ccccc2n3-c2ccc(-c3ccc(-n4c5ccccc5c5cc(-n6c7ccccc7c7ccccc76)ccc54)cc3)cc2)cc1] | 65 |
| A11 | 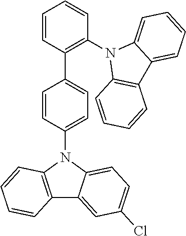 [SMILES: Clc1ccc2c(c1)c1ccccc1n2-c1ccc(-c2ccccc2-n2c3ccccc3c3ccccc32)cc1] | 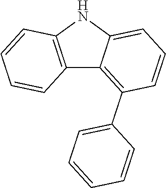 [SMILES: c1ccc(-c2cccc3[nH]c4ccccc4c23)cc1] | 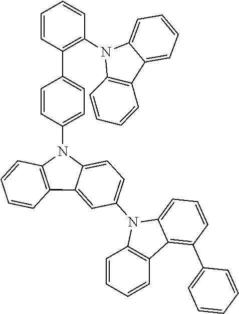 [SMILES: c1ccc(-c2cccc3c2c2ccccc2n3-c2ccc3c(c2)c2ccccc2n3-c2ccc(-c3ccccc3-n3c4ccccc4c4ccccc43)cc2)cc1] | 53 |
| A13 | 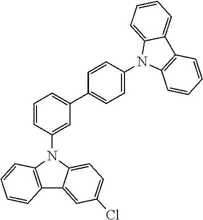 [SMILES: Clc1ccc2c(c1)c1ccccc1n2-c1cccc(-c2ccc(-n3c4ccccc4c4ccccc43)cc2)c1] | 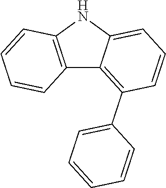 [SMILES: c1ccc(-c2cccc3[nH]c4ccccc4c23)cc1] | 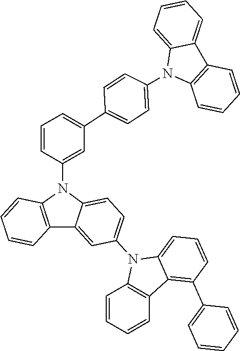 [SMILES: c1ccc(-c2cccc3c2c2ccccc2n3-c2ccc3c(c2)c2ccccc2n3-c2cccc(-c3ccc(-n4c5ccccc5c5ccccc54)cc3)c2)cc1] | 68 |
| A16 | 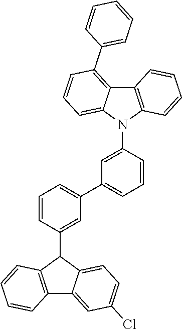 [SMILES: Clc1ccc2c(c1)-c1ccccc1C2c1cccc(-c2cccc(-n3c4ccccc4c4c(-c5ccccc5)cccc43)c2)c1] | 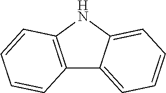 [SMILES: c1ccc2c(c1)[nH]c1ccccc12] | 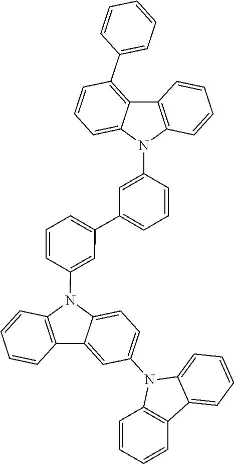 [SMILES: c1ccc(-c2cccc3c2c2ccccc2n3-c2cccc(-c3cccc(-n4c5ccccc5c5cc(-n6c7ccccc7c7ccccc76)ccc54)c3)c2)cc1] | 55 |
| A21 | 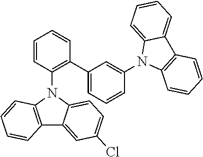 [SMILES: Clc1ccc2c(c1)c1ccccc1n2-c1ccccc1-c1cccc(-n2c3ccccc3c3ccccc32)c1] | 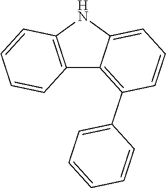 [SMILES: c1ccc(-c2cccc3[nH]c4ccccc4c23)cc1] | 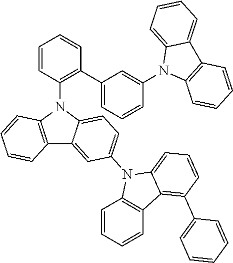 [SMILES: c1ccc(-c2cccc3c2c2ccccc2n3-c2ccc3c(c2)c2ccccc2n3-c2ccccc2-c2cccc(-n3c4ccccc4c4ccccc43)c2)cc1] | 60 |
| A24 | 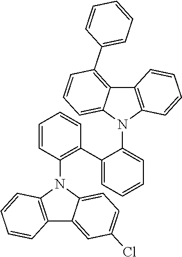 [SMILES: Clc1ccc2c(c1)c1ccccc1n2-c1ccccc1-c1ccccc1-n1c2ccccc2c2c(-c3ccccc3)cccc21] | 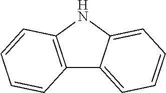 [SMILES: c1ccc2c(c1)[nH]c1ccccc12] | 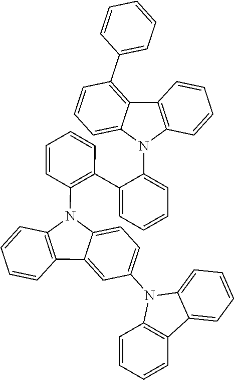 [SMILES: c1ccc(-c2cccc3c2c2ccccc2n3-c2ccccc2-c2ccccc2-n2c3ccccc3c3cc(-n4c5ccccc5c5ccccc54)ccc32)cc1] | 49 |
| A25 | 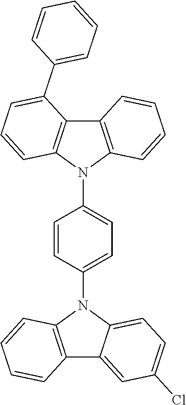 [SMILES: Clc1ccc2c(c1)c1ccccc1n2-c1ccc(-n2c3ccccc3c3c(-c4ccccc4)cccc32)cc1] | 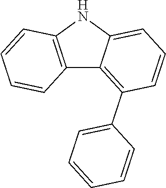 [SMILES: c1ccc(-c2cccc3[nH]c4ccccc4c23)cc1] | 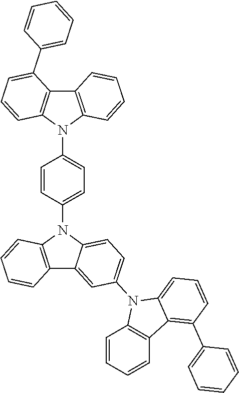 [SMILES: c1ccc(-c2cccc3c2c2ccccc2n3-c2ccc(-n3c4ccccc4c4cc(-n5c6ccccc6c6c(-c7ccccc7)cccc65)ccc43)cc2)cc1] | 70 |
| A26 | 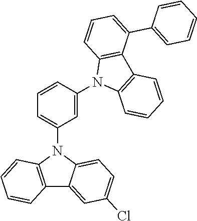 [SMILES: Clc1ccc2c(c1)c1ccccc1n2-c1cccc(-n2c3ccccc3c3c(-c4ccccc4)cccc32)c1] | 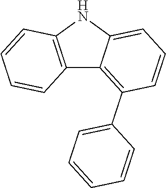 [SMILES: c1ccc(-c2cccc3[nH]c4ccccc4c23)cc1] | 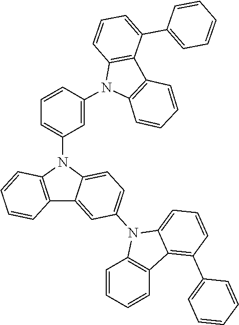 [SMILES: c1ccc(-c2cccc3c2c2ccccc2n3-c2cccc(-n3c4ccccc4c4cc(-n5c6ccccc6c6c(-c7ccccc7)cccc65)ccc43)c2)cc1] | 68 |
| A29 | 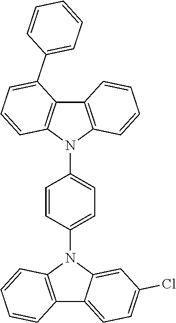 [SMILES: Clc1ccc2c3ccccc3n(-c3ccc(-n4c5ccccc5c5c(-c6ccccc6)cccc54)cc3)c2c1] | 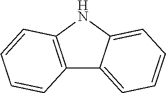 [SMILES: c1ccc2c(c1)[nH]c1ccccc12] | 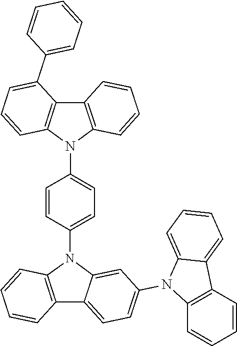 [SMILES: c1ccc(-c2cccc3c2c2ccccc2n3-c2ccc(-n3c4ccccc4c4ccc(-n5c6ccccc6c6ccccc65)cc43)cc2)cc1] | 65 |
| A31 | 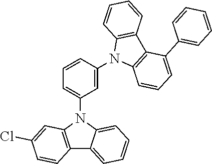 [SMILES: Clc1ccc2c3ccccc3n(-c3cccc(-n4c5ccccc5c5c(-c6ccccc6)cccc54)c3)c2c1] | 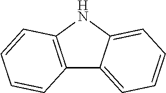 [SMILES: c1ccc2c(c1)[nH]c1ccccc12] | 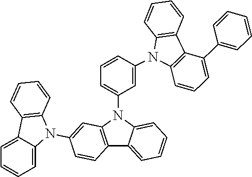 [SMILES: c1ccc(-c2cccc3c2c2ccccc2n3-c2cccc(-n3c4ccccc4c4ccc(-n5c6ccccc6c6ccccc65)cc43)c2)cc1] | 75 |
| A36 | 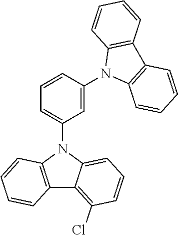 [SMILES: Clc1cccc2c1c1ccccc1n2-c1cccc(-n2c3ccccc3c3ccccc32)c1] | 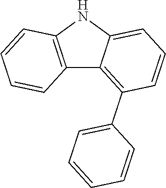 [SMILES: c1ccc(-c2cccc3[nH]c4ccccc4c23)cc1] | 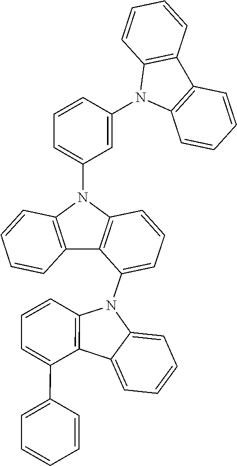 [SMILES: c1ccc(-c2cccc3c2c2ccccc2n3-c2cccc3c2c2ccccc2n3-c2cccc(-n3c4ccccc4c4ccccc43)c2)cc1] | 43 |
| A39 | 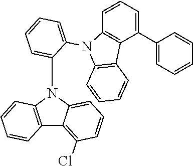 [SMILES: Clc1cccc2c1c1ccccc1n2-c1ccccc1-n1c2ccccc2c2c(-c3ccccc3)cccc21] | 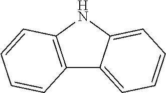 [SMILES: c1ccc2c(c1)[nH]c1ccccc12] | 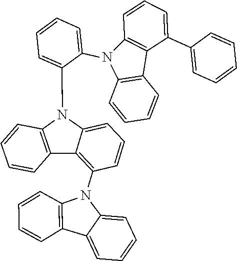 [SMILES: c1ccc(-c2cccc3c2c2ccccc2n3-c2ccccc2-n2c3ccccc3c3c(-n4c5ccccc5c5ccccc54)cccc32)cc1] | 36 |
| A47 | 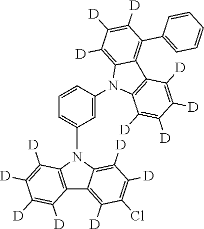 [SMILES: [2H]c1c([2H])c([2H])c2c(c1[2H])c1c([2H])c(Cl)c([2H])c([2H])c1n2-c1cccc(-n2c3c([2H])c([2H])c([2H])c([2H])c3c3c(-c4ccccc4)c([2H])c([2H])c([2H])c32)c1] | 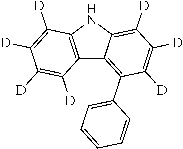 [SMILES: [2H]c1c([2H])c([2H])c2c([nH]c3c([2H])c([2H])c([2H])c(-c4ccccc4)c32)c1[2H]] | 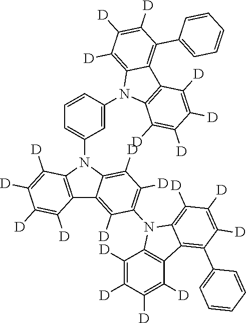 [SMILES: [2H]c1c([2H])c([2H])c2c(c1[2H])c1c([2H])c(-n3c4c([2H])c([2H])c([2H])c([2H])c4c4c(-c5ccccc5)c([2H])c([2H])c([2H])c43)c([2H])c([2H])c1n2-c1cccc(-n2c3c([2H])c([2H])c([2H])c([2H])c3c3c(-c4ccccc4)c([2H])c([2H])c([2H])c32)c1] | 72 |
| A48 | 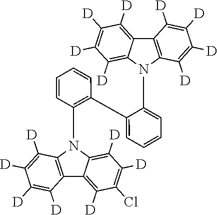 [SMILES: [2H]c1c([2H])c([2H])c2c(c1[2H])c1c([2H])c([2H])c([2H])c([2H])c1n2-c1ccccc1-c1ccccc1-n1c2c([2H])c([2H])c([2H])c([2H])c2c2c([2H])c(Cl)c([2H])c([2H])c21] | 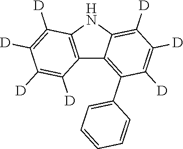 [SMILES: [2H]c1c([2H])c([2H])c2c([nH]c3c([2H])c([2H])c([2H])c(-c4ccccc4)c32)c1[2H]] | 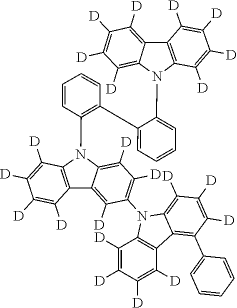 [SMILES: [2H]c1c([2H])c([2H])c2c(c1[2H])c1c([2H])c([2H])c([2H])c([2H])c1n2-c1ccccc1-c1ccccc1-n1c2c([2H])c([2H])c([2H])c([2H])c2c2c([2H])c(-n3c4c([2H])c([2H])c([2H])c([2H])c4c4c(-c5ccccc5)c([2H])c([2H])c([2H])c43)c([2H])c([2H])c21] | 52 |
| B1 | 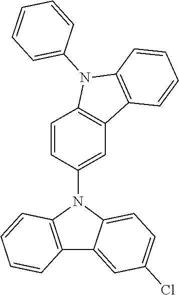 [SMILES: Clc1ccc2c(c1)c1ccccc1n2-c1ccc2c(c1)c1ccccc1n2-c1ccccc1] | 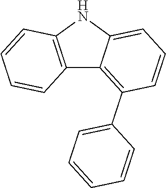 [SMILES: c1ccc(-c2cccc3[nH]c4ccccc4c23)cc1] | 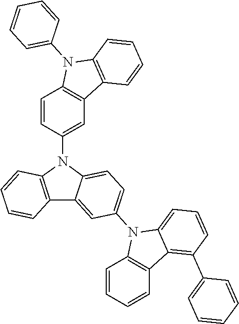 [SMILES: c1ccc(-c2cccc3c2c2ccccc2n3-c2ccc3c(c2)c2ccccc2n3-c2ccc3c(c2)c2ccccc2n3-c2ccccc2)cc1] | 77 |
| B2 | 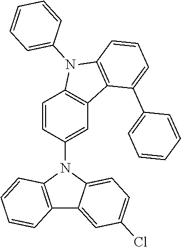 [SMILES: Clc1ccc2c(c1)c1ccccc1n2-c1ccc2c(c1)c1c(-c3ccccc3)cccc1n2-c1ccccc1] | 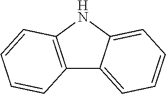 [SMILES: c1ccc2c(c1)[nH]c1ccccc12] | 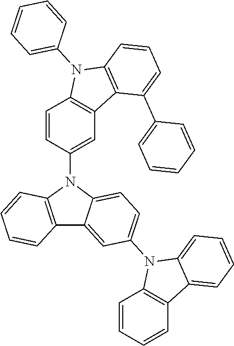 [SMILES: c1ccc(-c2cccc3c2c2cc(-n4c5ccccc5c5cc(-n6c7ccccc7c7ccccc76)ccc54)ccc2n3-c2ccccc2)cc1] | 65 |
| B4 | 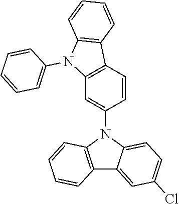 [SMILES: Clc1ccc2c(c1)c1ccccc1n2-c1ccc2c3ccccc3n(-c3ccccc3)c2c1] | 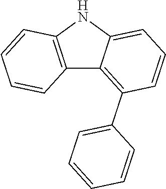 [SMILES: c1ccc(-c2cccc3[nH]c4ccccc4c23)cc1] | 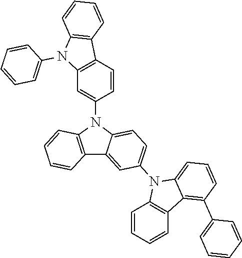 [SMILES: c1ccc(-c2cccc3c2c2ccccc2n3-c2ccc3c(c2)c2ccccc2n3-c2ccc3c4ccccc4n(-c4ccccc4)c3c2)cc1] | 59 |
| B5 | 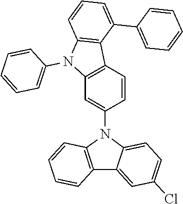 [SMILES: Clc1ccc2c(c1)c1ccccc1n2-c1ccc2c3c(-c4ccccc4)cccc3n(-c3ccccc3)c2c1] | 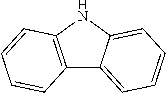 [SMILES: c1ccc2c(c1)[nH]c1ccccc12] | 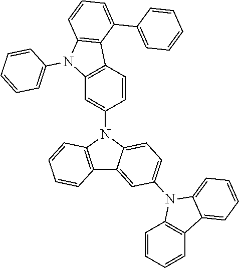 [SMILES: c1ccc(-c2cccc3c2c2ccc(-n4c5ccccc5c5cc(-n6c7ccccc7c7ccccc76)ccc54)cc2n3-c2ccccc2)cc1] | 74 |
| B18 | 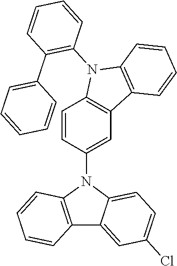 [SMILES: Clc1ccc2c(c1)c1ccccc1n2-c1ccc2c(c1)c1ccccc1n2-c1ccccc1-c1ccccc1] | 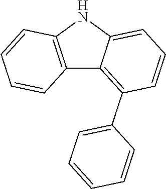 [SMILES: c1ccc(-c2cccc3[nH]c4ccccc4c23)cc1] | 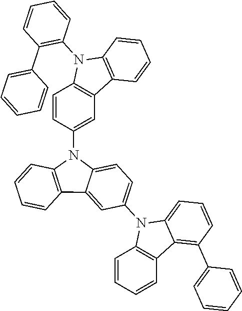 [SMILES: c1ccc(-c2ccccc2-n2c3ccccc3c3cc(-n4c5ccccc5c5cc(-n6c7ccccc7c7c(-c8ccccc8)cccc76)ccc54)ccc32)cc1] | 54 |
| B24 | 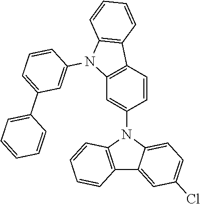 [SMILES: Clc1ccc2c(c1)c1ccccc1n2-c1ccc2c3ccccc3n(-c3cccc(-c4ccccc4)c3)c2c1] | 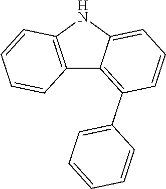 [SMILES: c1ccc(-c2cccc3[nH]c4ccccc4c23)cc1] | 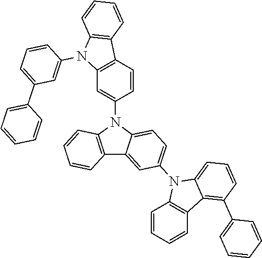 [SMILES: c1ccc(-c2cccc(-n3c4ccccc4c4ccc(-n5c6ccccc6c6cc(-n7c8ccccc8c8c(-c9ccccc9)cccc87)ccc65)cc43)c2)cc1] | 75 |
| B30 | 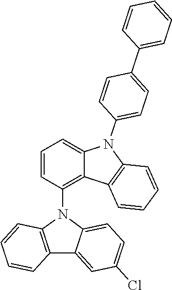 [SMILES: Clc1ccc2c(c1)c1ccccc1n2-c1cccc2c1c1ccccc1n2-c1ccc(-c2ccccc2)cc1] | 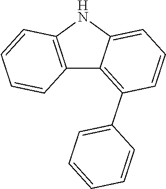 [SMILES: c1ccc(-c2cccc3[nH]c4ccccc4c23)cc1] | 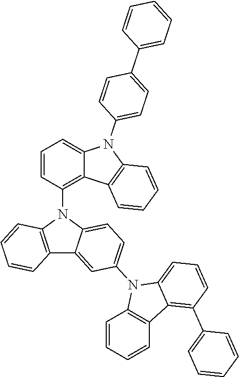 [SMILES: c1ccc(-c2ccc(-n3c4ccccc4c4c(-n5c6ccccc6c6cc(-n7c8ccccc8c8c(-c9ccccc9)cccc87)ccc65)cccc43)cc2)cc1] | 62 |
| B45 | 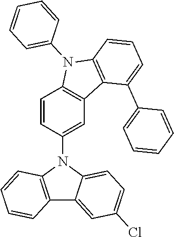 [SMILES: Clc1ccc2c(c1)c1ccccc1n2-c1ccc2c(c1)c1c(-c3ccccc3)cccc1n2-c1ccccc1] | 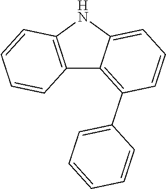 [SMILES: c1ccc(-c2cccc3[nH]c4ccccc4c23)cc1] | 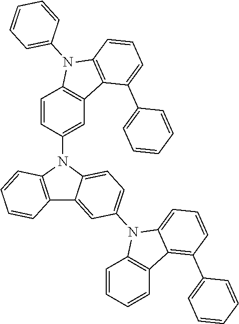 [SMILES: c1ccc(-c2cccc3c2c2ccccc2n3-c2ccc3c(c2)c2ccccc2n3-c2ccc3c(c2)c2c(-c4ccccc4)cccc2n3-c2ccccc2)cc1] | 61 |
| B46 | 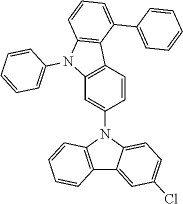 [SMILES: Clc1ccc2c(c1)c1ccccc1n2-c1ccc2c3c(-c4ccccc4)cccc3n(-c3ccccc3)c2c1] | 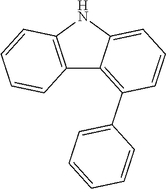 [SMILES: c1ccc(-c2cccc3[nH]c4ccccc4c23)cc1] | 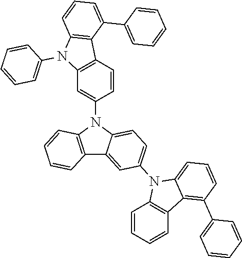 [SMILES: c1ccc(-c2cccc3c2c2ccccc2n3-c2ccc3c(c2)c2ccccc2n3-c2ccc3c4c(-c5ccccc5)cccc4n(-c4ccccc4)c3c2)cc1] | 64 |
| B50 | 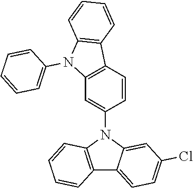 [SMILES: Clc1ccc2c3ccccc3n(-c3ccc4c5ccccc5n(-c5ccccc5)c4c3)c2c1] | 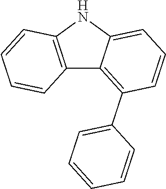 [SMILES: c1ccc(-c2cccc3[nH]c4ccccc4c23)cc1] | 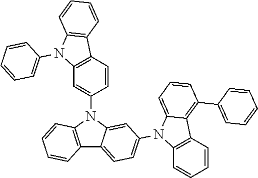 [SMILES: c1ccc(-c2cccc3c2c2ccccc2n3-c2ccc3c4ccccc4n(-c4ccc5c6ccccc6n(-c6ccccc6)c5c4)c3c2)cc1] | 68 |
| B53 | 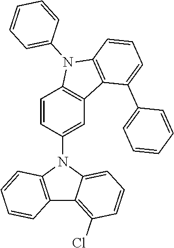 [SMILES: Clc1cccc2c1c1ccccc1n2-c1ccc2c(c1)c1c(-c3ccccc3)cccc1n2-c1ccccc1] | 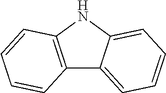 [SMILES: c1ccc2c(c1)[nH]c1ccccc12] | 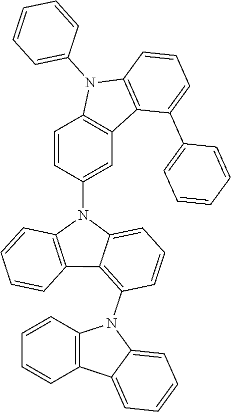 [SMILES: c1ccc(-c2cccc3c2c2cc(-n4c5ccccc5c5c(-n6c7ccccc7c7ccccc76)cccc54)ccc2n3-c2ccccc2)cc1] | 40 |
| B60 | 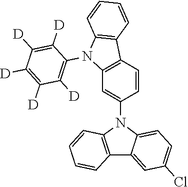 [SMILES: [2H]c1c([2H])c([2H])c(-n2c3ccccc3c3ccc(-n4c5ccccc5c5cc(Cl)ccc54)cc32)c([2H])c1[2H]] | 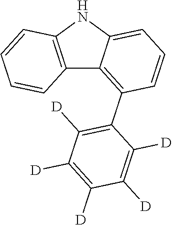 [SMILES: [2H]c1c([2H])c([2H])c(-c2cccc3[nH]c4ccccc4c23)c([2H])c1[2H]] | 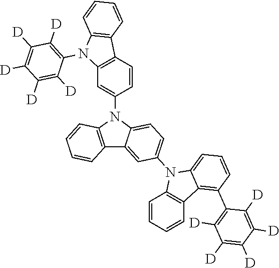 [SMILES: [2H]c1c([2H])c([2H])c(-c2cccc3c2c2ccccc2n3-c2ccc3c(c2)c2ccccc2n3-c2ccc3c4ccccc4n(-c4c([2H])c([2H])c([2H])c([2H])c4[2H])c3c2)c([2H])c1[2H]] | 74 |
| B62 | 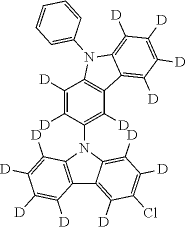 [SMILES: [2H]c1c([2H])c([2H])c2c(c1[2H])c1c([2H])c(-n3c4c([2H])c([2H])c([2H])c([2H])c4c4c([2H])c(Cl)c([2H])c([2H])c43)c([2H])c([2H])c1n2-c1ccccc1] | 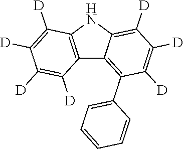 [SMILES: [2H]c1c([2H])c([2H])c2c([nH]c3c([2H])c([2H])c([2H])c(-c4ccccc4)c32)c1[2H]] | 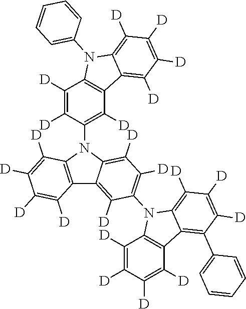 [SMILES: [2H]c1c([2H])c([2H])c2c(c1[2H])c1c([2H])c(-n3c4c([2H])c([2H])c([2H])c([2H])c4c4c([2H])c(-n5c6c([2H])c([2H])c([2H])c([2H])c6c6c(-c7ccccc7)c([2H])c([2H])c([2H])c65)c([2H])c([2H])c43)c([2H])c([2H])c1n2-c1ccccc1] | 60 |
| A51 | 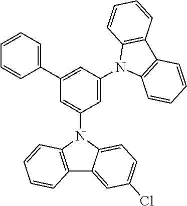 [SMILES: Clc1ccc2c(c1)c1ccccc1n2-c1cc(-c2ccccc2)cc(-n2c3ccccc3c3ccccc32)c1] | 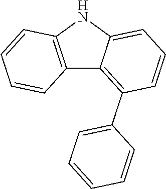 [SMILES: c1ccc(-c2cccc3[nH]c4ccccc4c23)cc1] | 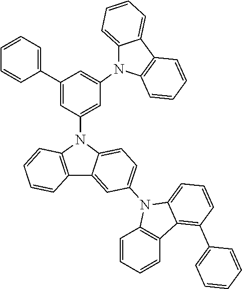 [SMILES: c1ccc(-c2cc(-n3c4ccccc4c4ccccc43)cc(-n3c4ccccc4c4cc(-n5c6ccccc6c6c(-c7ccccc7)cccc65)ccc43)c2)cc1] | 57 |
| B63 | 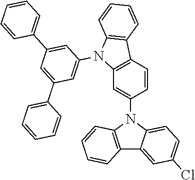 [SMILES: Clc1ccc2c(c1)c1ccccc1n2-c1ccc2c3ccccc3n(-c3cc(-c4ccccc4)cc(-c4ccccc4)c3)c2c1] | 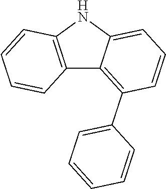 [SMILES: c1ccc(-c2cccc3[nH]c4ccccc4c23)cc1] | 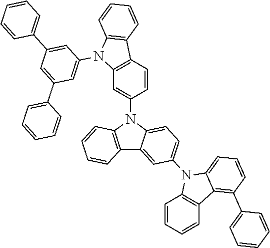 [SMILES: c1ccc(-c2cc(-c3ccccc3)cc(-n3c4ccccc4c4ccc(-n5c6ccccc6c6cc(-n7c8ccccc8c8c(-c9ccccc9)cccc87)ccc65)cc43)c2)cc1] | 63 |

Mass spectrum data for some compounds are shown in Table 4 below.

TABLE 4

| Compound | Mass spectrum data |
|---|---|
| Compound A1 | m/z = 650.3 (M + H)⁺ |
| Compound A3 | m/z = 650.3 (M + H)⁺ |
| Compound A4 | m/z = 650.3 (M + H)⁺ |
| Compound A8 | m/z = 726.3 (M + H)⁺ |
| Compound A11 | m/z = 726.3 (M + H)⁺ |
| Compound A13 | m/z = 726.3 (M + H)⁺ |
| Compound A16 | m/z = 726.3 (M + H)⁺ |
| Compound A21 | m/z = 726.3 (M + H)⁺ |
| Compound A24 | m/z = 726.3 (M + H)⁺ |
| Compound A25 | m/z = 726.3 (M + H)⁺ |
| Compound A26 | m/z = 726.3 (M + H)⁺ |
| Compound A29 | m/z = 650.3 (M + H)⁺ |
| Compound A31 | m/z = 650.3 (M + H)⁺ |
| Compound A36 | m/z = 650.3 (M + H)⁺ |
| Compound A39 | m/z = 650.3 (M + H)⁺ |
| Compound A51 | m/z = 726.3 (M + H)⁺ |
| Compound A47 | m/z = 747.4 (M + H)⁺ |
| Compound A48 | m/z = 748.4 (M + H)⁺ |
| Compound B1 | m/z = 650.3 (M + H)⁺ |
| Compound B2 | m/z = 650.3 (M + H)⁺ |
| Compound B4 | m/z = 650.3 (M + H)⁺ |
| Compound B5 | m/z = 650.3 (M + H)⁺ |
| Compound B18 | m/z = 726.3 (M + H)⁺ |
| Compound B24 | m/z = 726.3 (M + H)⁺ |
| Compound B30 | m/z = 726.3 (M + H)⁺ |
| Compound B45 | m/z = 726.3 (M + H)⁺ |
| Compound B46 | m/z = 726.3 (M + H)⁺ |
| Compound B50 | m/z = 650.3 (M + H)⁺ |
| Compound B53 | m/z = 650.3 (M + H)⁺ |
| Compound B60 | m/z = 660.3 (M + H)⁺ |
| Compound B62 | m/z = 671.4 (M + H)⁺ |
| Compound B63 | m/z = 802.3 (M + H)⁺ |

NMR data of some compounds are shown in Table 5 below

TABLE 5

| Compound | NMR data |
|---|---|
| Compound A1 | ¹H-NMR (CD₂Cl₂, 400 MHz): 8.26 (s, 1H), 8.20 (d, 2H), 8.12 (d, 1H), |
| | 7.82-7.84 (m, 4H), 7.60-7.53 (m, 6H), 7.50-7.38 (m, 11H), 7.36-7.29 |
| | (m, 4H), 7.19-7.15 (m, 2H). |
| Compound B1 | ¹H-NMR (CD₂Cl₂, 400 MHz): 8.34 (s, 1H), 8.28 (s, 1H), 8.23 (d, 1H), |
| | 8.15 (d, 1H), 7.68-7.62 (m, 5H), 7.59-7.48 (m, 9H), 7.45-7.31 (m, 10H), |
| | 7.29 (t, 1H), 7.19-7.16 (m, 2H). |

### Manufacture of Organic Electroluminescent Device

### Example 1: Manufacture of Blue Organic Electroluminescent Device

Devices were Manufactured by Following Process

Surface treatment was performed with UV, ozone and O₂:N₂ plasma on an ITO/Ag/ITO experimental substrate with a thickness of 100 Å/1000 Å/100 Å to increase the work function of an anode, and the surface of the experimental substrate can be cleaned with an organic solvent to remove impurities and oil from the surface of the experimental substrate.

A compound HT-27 and PD were co-evaporated on the experimental substrate at an evaporation rate ratio of 98%:2% to form a hole injection layer with a thickness of 100 Å, and then a compound HT-27 was evaporated on the hole injection layer to form a hole transport layer with a thickness of 1065 Å.

A compound HT-29 was evaporated on the hole transport layer to form an electron blocking layer with a thickness of 50 Å.

A compound A1, a compound BH—N, and BD-01 were co-evaporated on the electron blocking layer at an evaporation rate ratio of 70%:30%:10% to form an organic light-emitting layer with a thickness of 350 Å.

A compound ET-1 and LiQ were co-evaporated on the organic light-emitting layer at an evaporation rate ratio of 50%:50% to form an electron transport layer with a thickness of 350 Å.

Yb was evaporated on the electron transport layer to form an electron injection layer with a thickness of 15 Å; and then magnesium (Mg) and silver (Ag) were co-evaporated on the electron injection layer at an evaporation rate ratio of 10%:90% to form a cathode with a thickness of 130 Å.

A compound CP-1 was evaporated on the cathode to form an organic capping layer with a thickness of 680 Å, thus completing the manufacture of a blue organic electroluminescent device.

### Examples 2 to 32

An organic electroluminescent device was manufactured by the same method as that in Example 1 except that Compound X in Table 7 were used instead of the Compound A1 in Example 1 when the organic light-emitting layer was manufactured.

### Comparative Examples 1-4

An organic electroluminescent device was manufactured by the same method as that in Example 1 except that a Compound 1, a Compound 2, a Compound 3, or a Compound 4 in Table 6 was used instead of the Compound A1 in Example 1 when the organic light-emitting layer was manufactured.

When the devices in the Examples and Comparative examples were manufactured, structures of compounds used are listed in Table 6 below:

TABLE 6

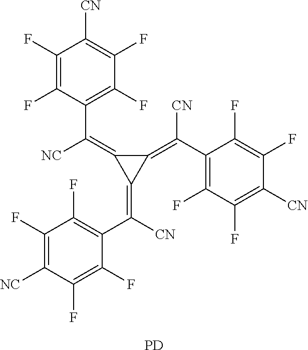 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; "PD"]

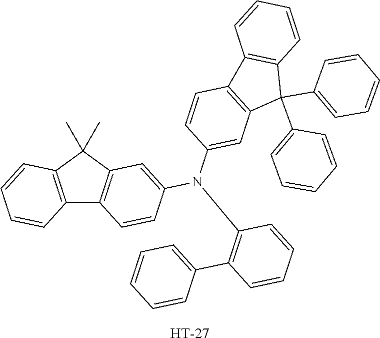

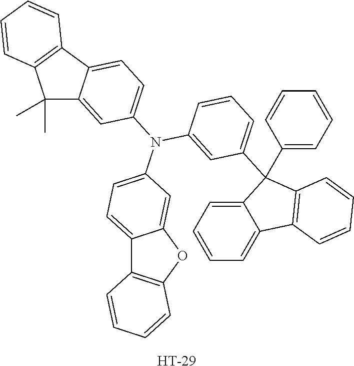

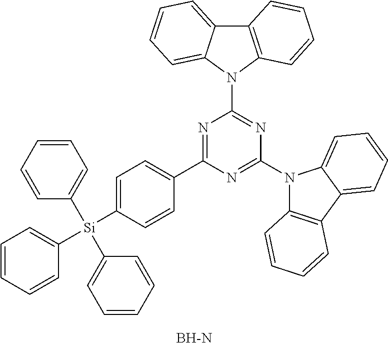

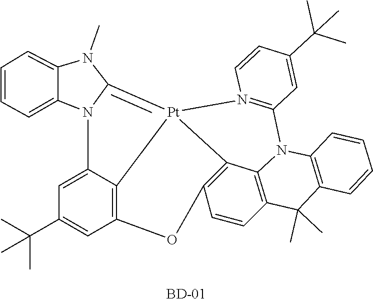

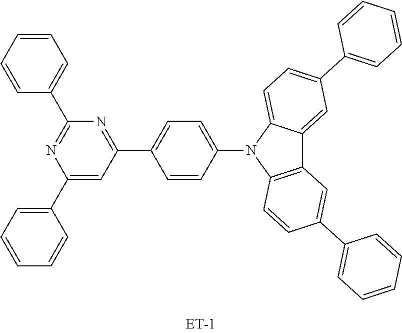 [SMILES: c1ccc(-c2ccc3c(c2)c2cc(-c4ccccc4)ccc2n3-c2ccc(-c3cc(-c4ccccc4)nc(-c4ccccc4)n3)cc2)cc1]

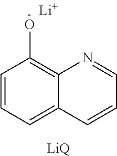

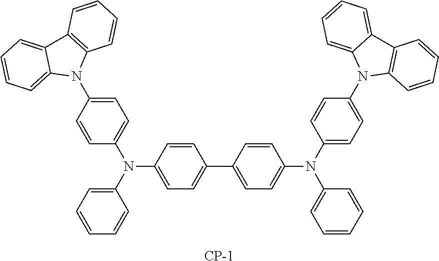 [SMILES: c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1 ; "CP-1"]

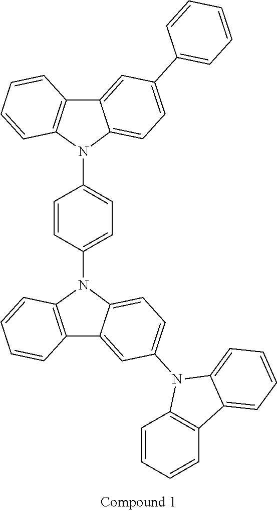 [SMILES: c1ccc(-c2ccc3c(c2)c2ccccc2n3-c2ccc(-n3c4ccccc4c4cc(-n5c6ccccc6c6ccccc65)ccc43)cc2)cc1]

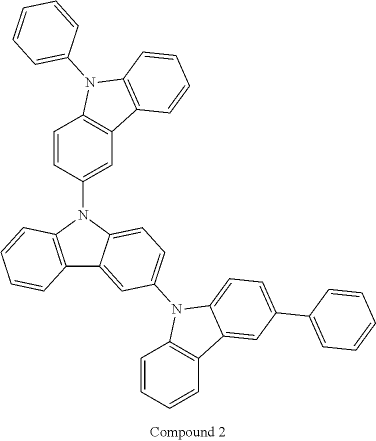 [SMILES: c1ccc(-c2ccc3c(c2)c2ccccc2n3-c2ccc3c(c2)c2ccccc2n3-c2ccc3c(c2)c2ccccc2n3-c2ccccc2)cc1]

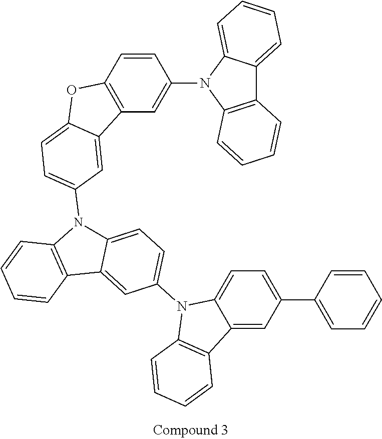 [SMILES: c1ccc(-c2ccc3c(c2)c2ccccc2n3-c2ccc3c(c2)c2ccccc2n3-c2ccc3oc4ccc(-n5c6ccccc6c6ccccc65)cc4c3c2)cc1]

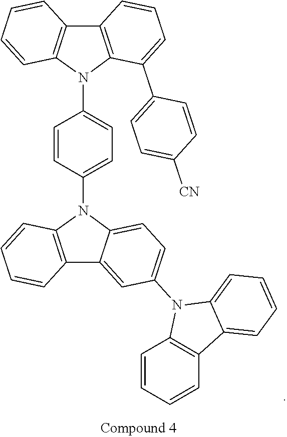 [SMILES: N#Cc1ccc(-c2cccc3c4ccccc4n(-c4ccc(-n5c6ccccc6c6cc(-n7c8ccccc8c8ccccc87)ccc65)cc4)c23)cc1]

The blue organic electroluminescent devices manufactured in Examples 1 to 32 and Comparative examples 1 to 4 were subjected to performance tests, and specifically, the current-voltage-brightness (IVL) and T90 device service life were tested at a brightness of 1000 nit, and the test results are shown in Table 7 below:

TABLE 7

| | | Operating | | Chroma- | Service |
|---|---|---|---|---|---|
| | | voltage | Current | ticity | life |
| Example | | Volt | efficiency | coordinate | T90 |
| No. | Compound X | (V) | (Cd/A) | CIEx, y | (hrs) |
| Example 1 | Compound A1 | 3.93 | 16.10 | 0.14, 0.05 | 162 |
| Example 2 | Compound A3 | 3.95 | 16.38 | 0.14, 0.05 | 168 |
| Example 3 | Compound A4 | 3.96 | 15.57 | 0.14, 0.05 | 168 |
| Example 4 | Compound A8 | 4.04 | 15.41 | 0.14, 0.05 | 158 |
| Example 5 | Compound A11 | 3.96 | 16.40 | 0.14, 0.05 | 165 |
| Example 6 | Compound A13 | 4.05 | 15.31 | 0.14, 0.05 | 162 |
| Example 7 | Compound A16 | 3.96 | 15.90 | 0.14, 0.05 | 164 |
| Example 8 | Compound A21 | 4.00 | 15.30 | 0.14, 0.05 | 168 |
| Example 9 | Compound A24 | 3.97 | 15.11 | 0.14, 0.05 | 161 |
| Example 10 | Compound A25 | 3.97 | 15.12 | 0.14, 0.05 | 163 |
| Example 11 | Compound A26 | 3.96 | 15.99 | 0.14, 0.05 | 169 |
| Example 12 | Compound A29 | 3.94 | 15.88 | 0.14, 0.05 | 161 |
| Example 13 | Compound A31 | 4.03 | 16.00 | 0.14, 0.05 | 167 |
| Example 14 | Compound A36 | 3.99 | 15.58 | 0.14, 0.05 | 161 |
| Example 15 | Compound A39 | 3.93 | 15.62 | 0.14, 0.05 | 158 |
| Example 16 | Compound A47 | 3.97 | 16.41 | 0.14, 0.05 | 177 |
| Example 17 | Compound A48 | 3.96 | 16.37 | 0.14, 0.05 | 176 |
| Example 18 | Compound B1 | 3.93 | 16.20 | 0.14, 0.05 | 143 |
| Example 19 | Compound B2 | 3.95 | 15.39 | 0.14, 0.05 | 139 |
| Example 20 | Compound B4 | 3.94 | 16.43 | 0.14, 0.05 | 145 |
| Example 21 | Compound B5 | 3.97 | 16.42 | 0.14, 0.05 | 138 |
| Example 22 | Compound B18 | 3.98 | 15.60 | 0.14, 0.05 | 140 |
| Example 23 | Compound B24 | 3.91 | 15.42 | 0.14, 0.05 | 142 |
| Example 24 | Compound B30 | 3.96 | 15.40 | 0.14, 0.05 | 137 |
| Example 25 | Compound B45 | 4.01 | 15.80 | 0.14, 0.05 | 141 |
| Example 26 | Compound B46 | 4.00 | 16.31 | 0.14, 0.05 | 142 |
| Example 27 | Compound B50 | 3.92 | 15.10 | 0.14, 0.05 | 141 |
| Example 28 | Compound B53 | 3.91 | 15.50 | 0.14, 0.05 | 143 |
| Example 29 | Compound B60 | 3.98 | 16.30 | 0.14, 0.05 | 144 |
| Example 30 | Compound B62 | 4.05 | 16.03 | 0.14, 0.05 | 149 |
| Example 31 | Compound B63 | 3.99 | 15.70 | 0.14, 0.05 | 140 |
| Example 32 | Compound A51 | 4.02 | 15.20 | 0.14, 0.05 | 157 |
| Comparative | Compound 1 | 4.06 | 13.30 | 0.14, 0.05 | 102 |
| example 1 | | | | | |
| Comparative | Compound 2 | 4.08 | 11.60 | 0.14, 0.05 | 118 |
| example 2 | | | | | |
| Comparative | Compound 3 | 4.14 | 12.90 | 0.14, 0.05 | 109 |
| example 3 | | | | | |
| Comparative | Compound 4 | 4.23 | 13.10 | 0.14, 0.05 | 97 |
| example 4 | | | | | |

As can be seen from the above Table 7, when the compound of the present disclosure is used as a host material of the organic light-emitting layer of the blue organic electroluminescent device, the performance of the device can be greatly improved. In particular, the efficiency of the organic electroluminescent devices in Examples 1 to 32 is improved by at least 13.5% and the service life T90 of the organic electroluminescent devices in Examples 1 to 32 is improved by at least 16.1% compared with the organic electroluminescent devices in Comparative examples 1 to 4.

Some embodiments of the present disclosure are described in detail above with reference to the drawings, however, the present disclosure is not limited to the specific details in the above embodiments, in the technical concept range of the present disclosure, the technical solution of the present disclosure can be subjected to various simple variations, and these simple variations all belong to the protection range of the present disclosure.

1. A nitrogen-containing compound, having a structure represented by a Formula 1: 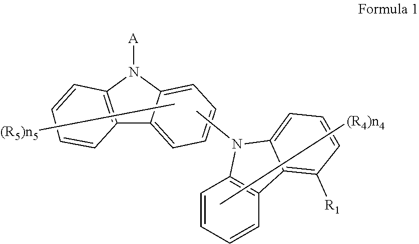
wherein R₁ is selected from a hydrogen, a deuterium, an aryl with 6 to 12 carbon atoms or a deuteroaryl with 6 to 12 carbon atoms;
A is selected from groups represented by a Formula 1-1; 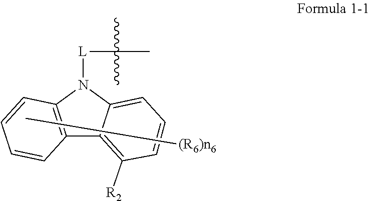
R₂ is selected from a hydrogen, a deuterium, an aryl with 6 to 12 carbon atoms or a deuteroaryl with 6 to 12 carbon atoms, and at least one of R₁ and R₂ is selected from an aryl with 6 to 12 carbon atoms or a deuteroaryl with 6 to 12 carbon atoms;
or, A is selected from groups represented by a Formula 1-2; 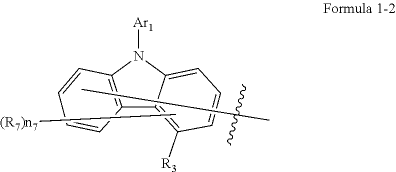
R₃ is selected from a hydrogen, a deuterium, an aryl with 6 to 12 carbon atoms or a deuteroaryl with 6 to 12 carbon atoms, and at least one of R₁ and R₃ is selected from an aryl with 6 to 12 carbon atoms or a deuteroaryl with 6 to 12 carbon atoms;
each R₄, each R₅, each R₆ and each R₇ are the same or different, and are respectively and independently selected from a deuterium, an aryl with 6 to 12 carbon atoms or a deuteroaryl with 6 to 12 carbon atoms;
n₄ is the number of R₄, and is selected from 0, 1, 2, 3, 4, 5, 6 or 7, and in the case where n₄ is greater than 1, each R₄ is the same or different;
n₅ is the number of R₅, and is selected from 0, 1, 2, 3, 4, 5, 6 or 7, and in the case where n₅ is greater than 1, each R₅ is the same or different;
no is the number of R₆, and is selected from 0, 1, 2, 3, 4, 5, 6 or 7, and in the case where no is greater than 1, each R₆ is the same or different;
n₇ is the number of R₇, and is selected from 0, 1, 2, 3, 4, 5 or 6, and in the case where n₇ is greater than 1, each R₇ is the same or different;
L is selected from a substituted or unsubstituted arylene with 6 to 12 carbon atoms;
substituent(s) of L are the same or different, and are respectively and independently selected from a deuterium, a phenyl or a pentadeuterophenyl;
Ar₁ is selected from a substituted or unsubstituted aryl with 6 to 30 carbon atoms; and
substituent(s) of Ar₁ are the same or different, and are respectively and independently selected from a deuterium, a halogen group, a cyano, an alkyl with 1 to 10 carbon atoms, a cycloalkyl with 3 to 10 carbon atoms, an aryl with 6 to 12 carbon atoms, a deuteroaryl with 6 to 12 carbon atoms, or a haloaryl with 6 to 12 carbon atoms.

2. The nitrogen-containing compound according to claim 1, wherein the nitrogen-containing compound is selected from compounds represented by a Formula C or a Formula B: 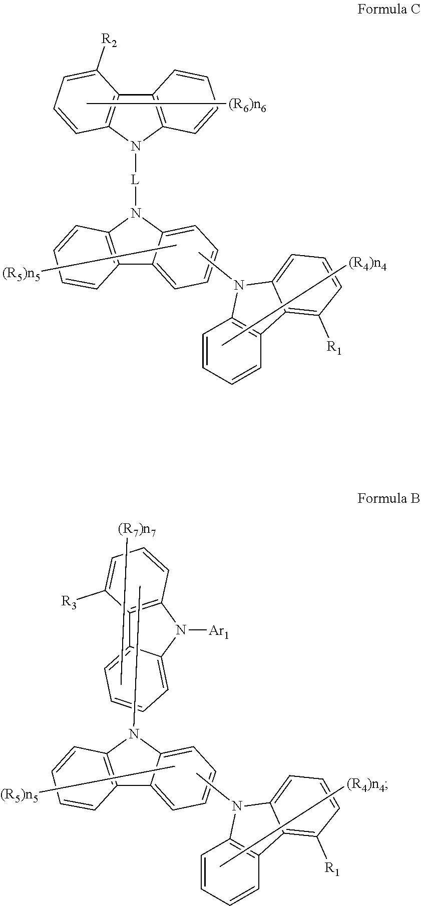
in the formula C, R₁ and R₂ are the same or different, and are respectively and independently selected from a hydrogen, a deuterium, an aryl with 6 to 12 carbon atoms or a deuteroaryl with 6 to 12 carbon atoms, and at least one of R₁ and R₂ is an aryl with 6 to 12 carbon atoms or a deuteroaryl with 6 to 12 carbon atoms; and
in the formula B, R₁ and R₃ are the same or different, and are respectively and independently selected from a hydrogen, a deuterium, an aryl with 6 to 12 carbon atoms or a deuteroaryl with 6 to 12 carbon atoms, and at least one of R₁ and R₃ is an aryl with 6 to 12 carbon atoms or a deuteroaryl with 6 to 12 carbon atoms.

3. The nitrogen-containing compound according to claim 1, wherein L is selected from a substituted or unsubstituted phenylene or a substituted or unsubstituted biphenylene.

4. The nitrogen-containing compound according to claim 1, wherein L is selected from the group consisting of:

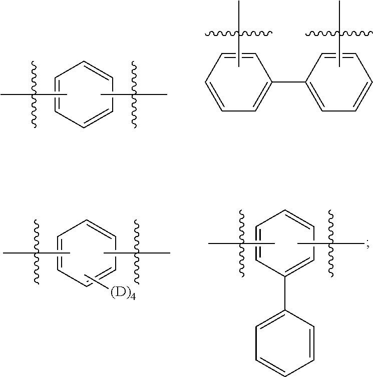

5. The nitrogen-containing compound according to claim 1, wherein Ar₁ is selected from a substituted or unsubstituted phenyl, a substituted or unsubstituted biphenyl, a substituted or unsubstituted phenanthryl, a substituted or unsubstituted naphthyl, a substituted or unsubstituted terphenyl, or a substituted or unsubstituted fluorenyl.

6. The nitrogen-containing compound according to claim 1, wherein Ar₁ is selected from the group consisting of:

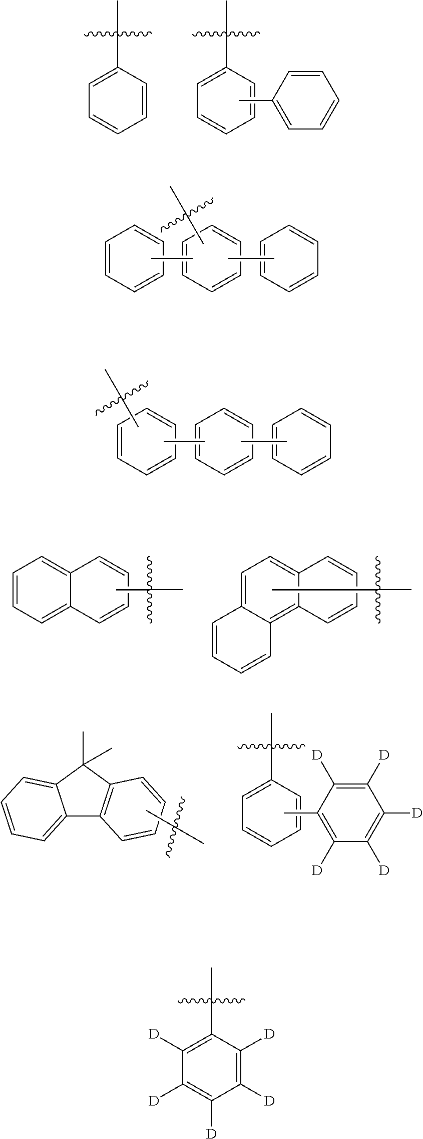

7. The nitrogen-containing compound according to claim 1, wherein R₁ and R₂ are the same or different, and are respectively and independently selected from a hydrogen, a deuterium, a phenyl or a pentadeuterophenyl, and at least one of R₁ and R₂ is a phenyl or a pentadeuterophenyl.

8. The nitrogen-containing compound according to claim 1, wherein R₁ and R₃ are the same or different, and are respectively and independently selected from a hydrogen, a deuterium, a phenyl or a pentadeuterophenyl, and at least one of R₁ and R₃ is a phenyl or a pentadeuterophenyl.

9. The nitrogen-containing compound according to claim 1, wherein the nitrogen-containing compound is selected from the group consisting of the following compounds:

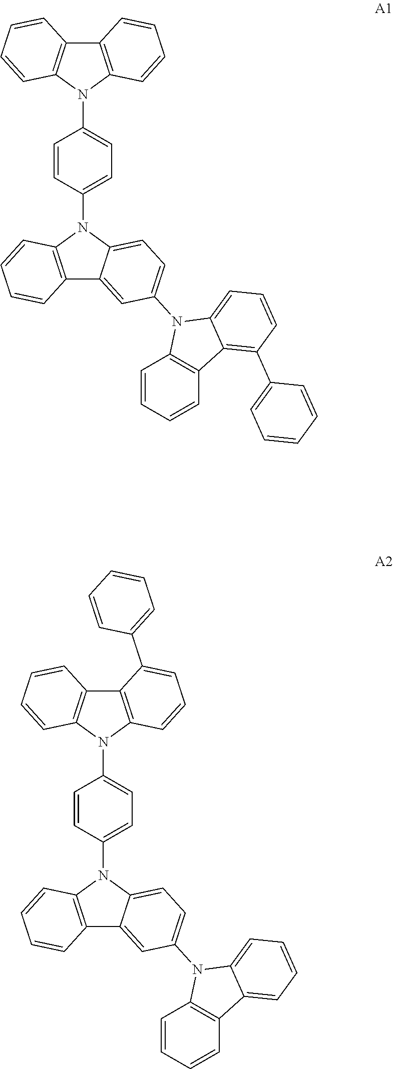 [SMILES: c1ccc(-c2cccc3c2c2ccccc2n3-c2ccc3c(c2)c2ccccc2n3-c2ccc(-n3c4ccccc4c4ccccc43)cc2)cc1 ; c1ccc(-c2cccc3c2c2ccccc2n3-c2ccc(-n3c4ccccc4c4cc(-n5c6ccccc6c6ccccc65)ccc43)cc2)cc1]

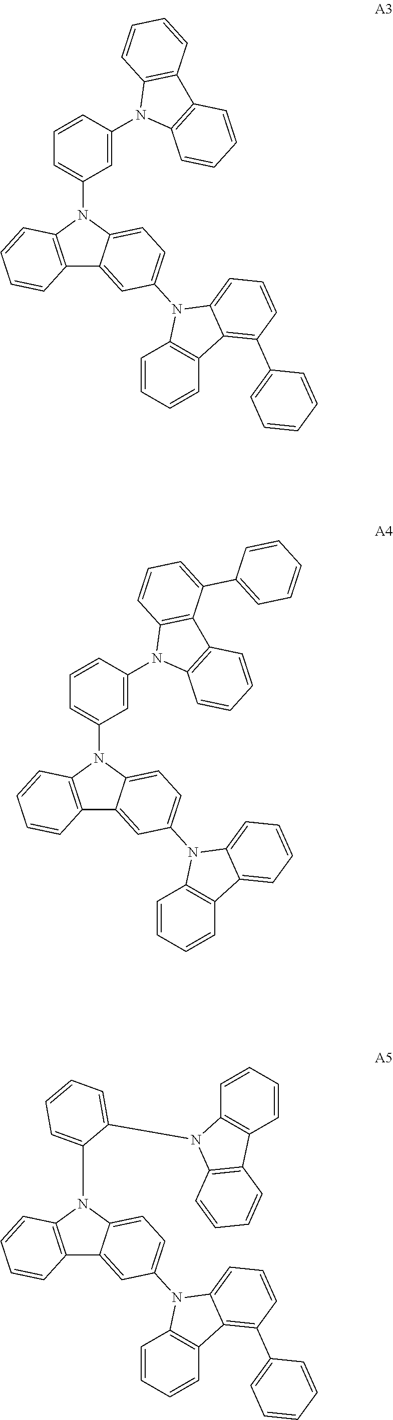 [SMILES: c1ccc(-c2cccc3c2c2ccccc2n3-c2ccc3c(c2)c2ccccc2n3-c2cccc(-n3c4ccccc4c4ccccc43)c2)cc1 ; c1ccc(-c2cccc3c2c2ccccc2n3-c2cccc(-n3c4ccccc4c4cc(-n5c6ccccc6c6ccccc65)ccc43)c2)cc1 ; c1ccc(-c2cccc3c2c2ccccc2n3-c2ccc3c(c2)c2ccccc2n3-c2ccccc2-n2c3ccccc3c3ccccc32)cc1]

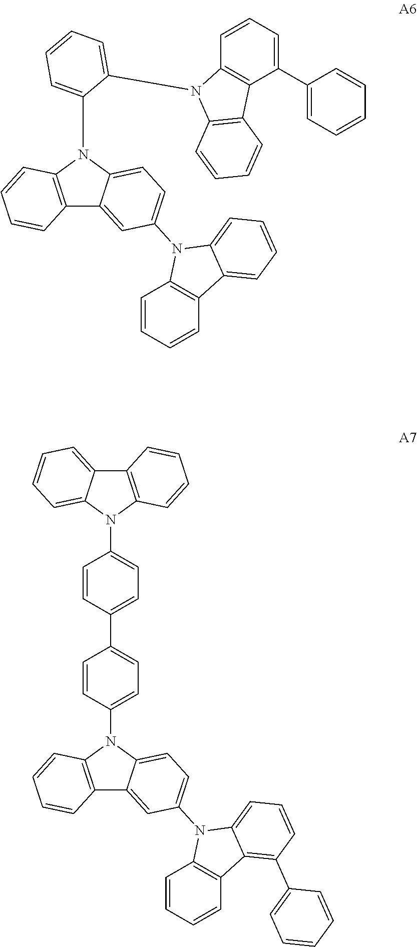 [SMILES: c1ccc(-c2cccc3c2c2ccccc2n3-c2ccccc2-n2c3ccccc3c3cc(-n4c5ccccc5c5ccccc54)ccc32)cc1 ; c1ccc(-c2cccc3c2c2ccccc2n3-c2ccc3c(c2)c2ccccc2n3-c2ccc(-c3ccc(-n4c5ccccc5c5ccccc54)cc3)cc2)cc1]

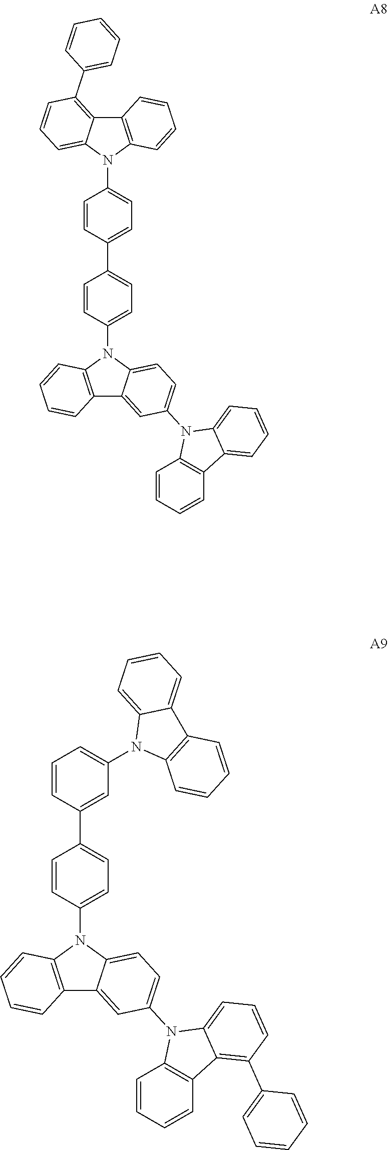 [SMILES: c1ccc(-c2cccc3c2c2ccccc2n3-c2ccc(-c3ccc(-n4c5ccccc5c5cc(-n6c7ccccc7c7ccccc76)ccc54)cc3)cc2)cc1 ; c1ccc(-c2cccc3c2c2ccccc2n3-c2ccc3c(c2)c2ccccc2n3-c2ccc(-c3cccc(-n4c5ccccc5c5ccccc54)c3)cc2)cc1]

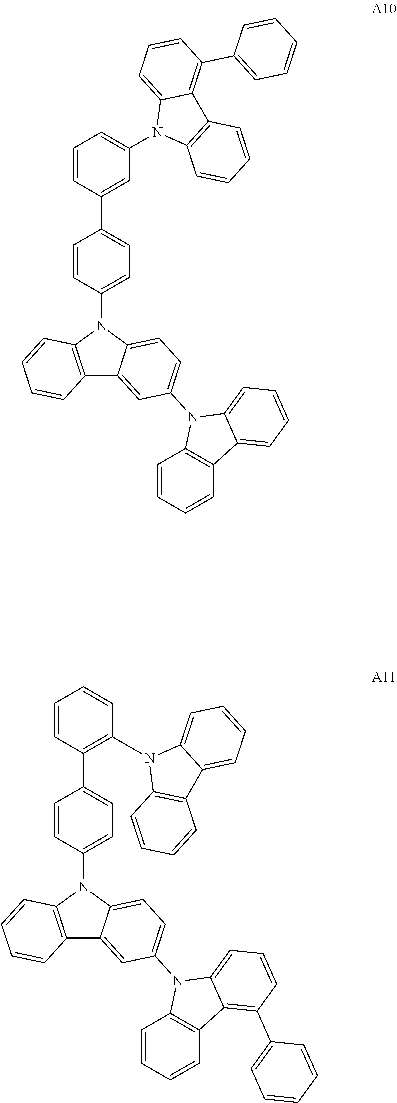 [SMILES: c1ccc(-c2cccc3c2c2ccccc2n3-c2cccc(-c3ccc(-n4c5ccccc5c5cc(-n6c7ccccc7c7ccccc76)ccc54)cc3)c2)cc1 ; c1ccc(-c2cccc3c2c2ccccc2n3-c2ccc3c(c2)c2ccccc2n3-c2ccc(-c3ccccc3-n3c4ccccc4c4ccccc43)cc2)cc1]

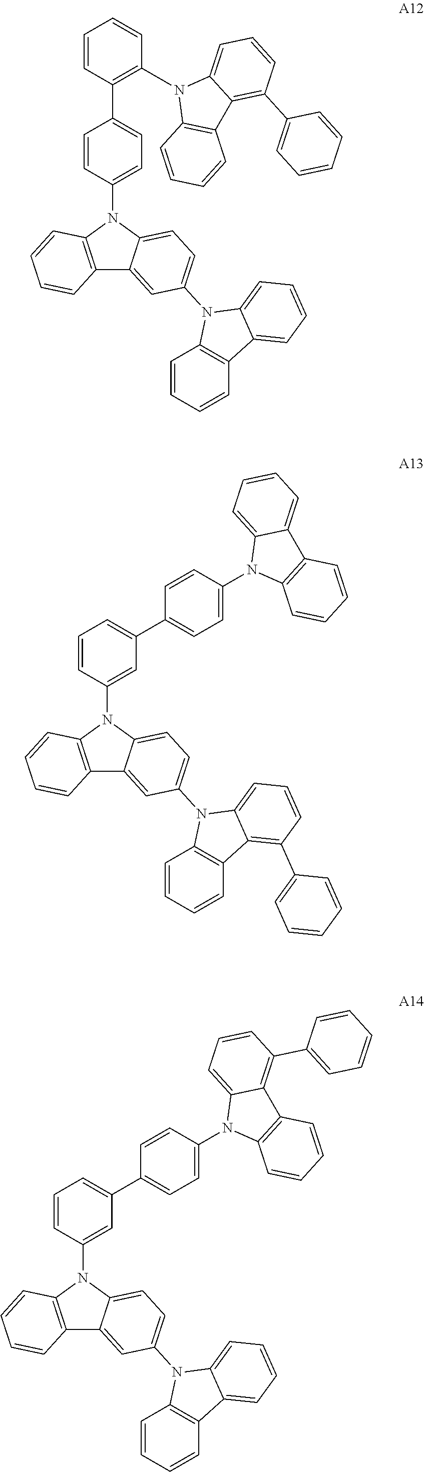 [SMILES: c1ccc(-c2cccc3c2c2ccccc2n3-c2ccccc2-c2ccc(-n3c4ccccc4c4cc(-n5c6ccccc6c6ccccc65)ccc43)cc2)cc1 ; c1ccc(-c2cccc3c2c2ccccc2n3-c2ccc3c(c2)c2ccccc2n3-c2cccc(-c3ccc(-n4c5ccccc5c5ccccc54)cc3)c2)cc1 ; c1ccc(-c2cccc3c2c2ccccc2n3-c2ccc(-c3cccc(-n4c5ccccc5c5cc(-n6c7ccccc7c7ccccc76)ccc54)c3)cc2)cc1]

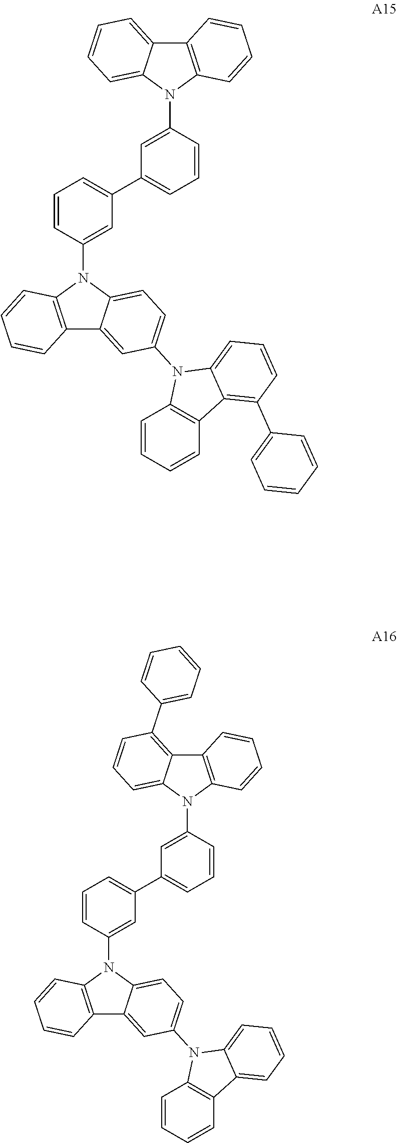 [SMILES: c1ccc(-c2cccc3c2c2ccccc2n3-c2ccc3c(c2)c2ccccc2n3-c2cccc(-c3cccc(-n4c5ccccc5c5ccccc54)c3)c2)cc1 ; c1ccc(-c2cccc3c2c2ccccc2n3-c2cccc(-c3cccc(-n4c5ccccc5c5cc(-n6c7ccccc7c7ccccc76)ccc54)c3)c2)cc1]

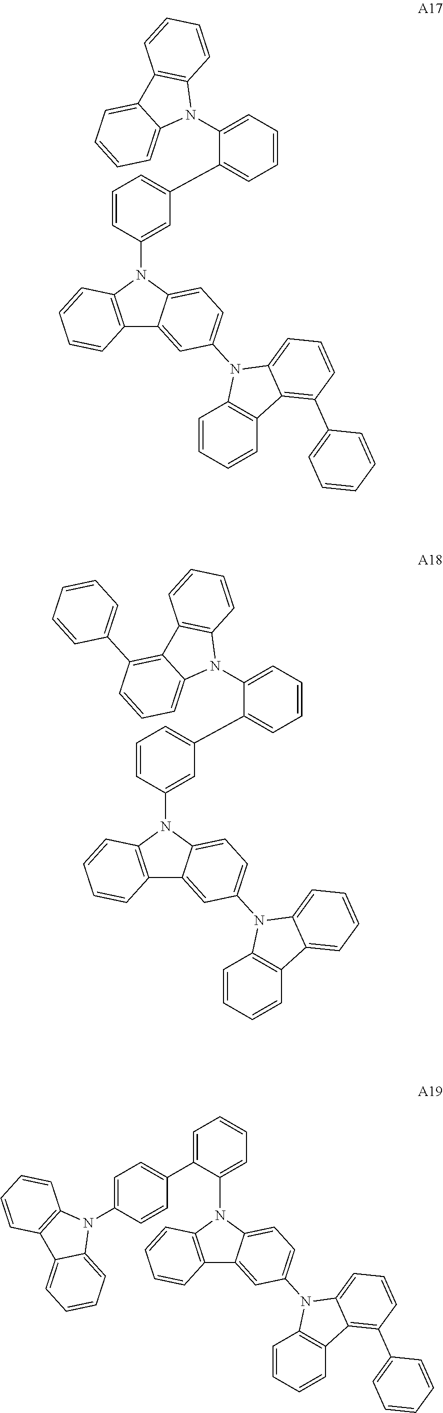 [SMILES: c1ccc(-c2cccc3c2c2ccccc2n3-c2ccc3c(c2)c2ccccc2n3-c2cccc(-c3ccccc3-n3c4ccccc4c4ccccc43)c2)cc1 ; c1ccc(-c2cccc3c2c2ccccc2n3-c2ccccc2-c2cccc(-n3c4ccccc4c4cc(-n5c6ccccc6c6ccccc65)ccc43)c2)cc1 ; c1ccc(-c2cccc3c2c2ccccc2n3-c2ccc3c(c2)c2ccccc2n3-c2ccccc2-c2ccc(-n3c4ccccc4c4ccccc43)cc2)cc1]

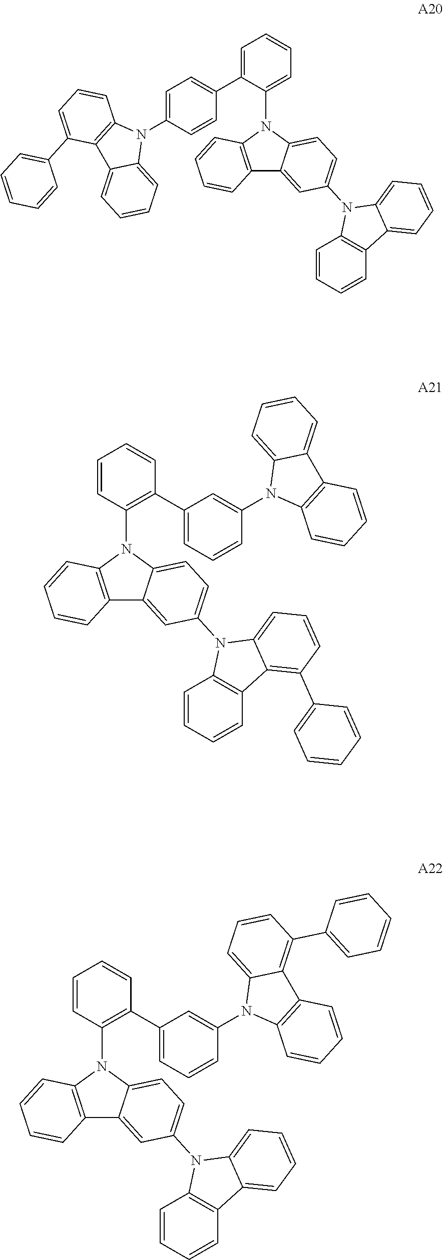 [SMILES: c1ccc(-c2cccc3c2c2ccccc2n3-c2ccc(-c3ccccc3-n3c4ccccc4c4cc(-n5c6ccccc6c6ccccc65)ccc43)cc2)cc1 ; c1ccc(-c2cccc3c2c2ccccc2n3-c2ccc3c(c2)c2ccccc2n3-c2ccccc2-c2cccc(-n3c4ccccc4c4ccccc43)c2)cc1 ; c1ccc(-c2cccc3c2c2ccccc2n3-c2cccc(-c3ccccc3-n3c4ccccc4c4cc(-n5c6ccccc6c6ccccc65)ccc43)c2)cc1]

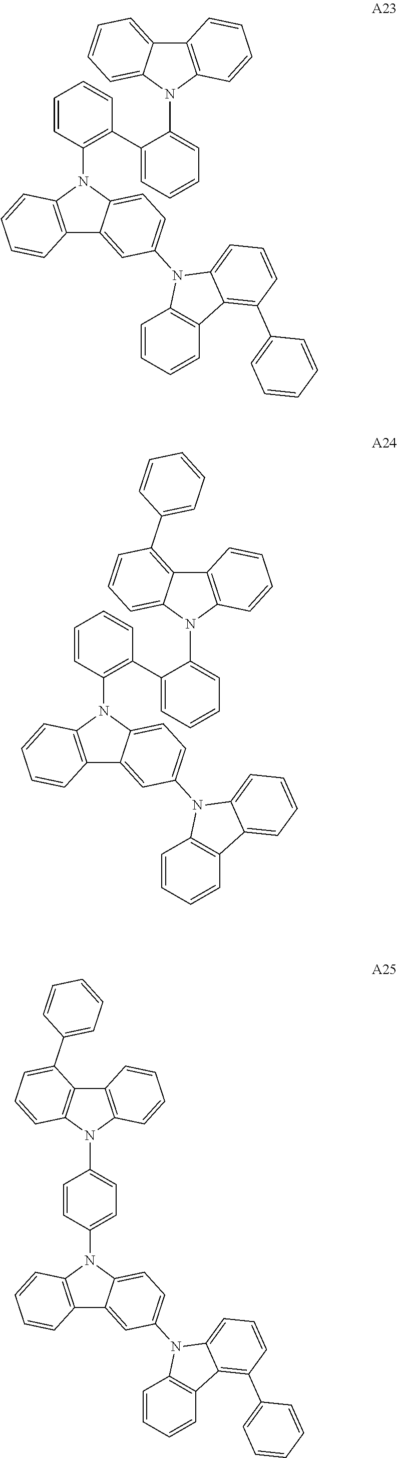 [SMILES: c1ccc(-c2cccc3c2c2ccccc2n3-c2ccc3c(c2)c2ccccc2n3-c2ccccc2-c2ccccc2-n2c3ccccc3c3ccccc32)cc1 ; c1ccc(-c2cccc3c2c2ccccc2n3-c2ccccc2-c2ccccc2-n2c3ccccc3c3cc(-n4c5ccccc5c5ccccc54)ccc32)cc1 ; c1ccc(-c2cccc3c2c2ccccc2n3-c2ccc(-n3c4ccccc4c4cc(-n5c6ccccc6c6c(-c7ccccc7)cccc65)ccc43)cc2)cc1]

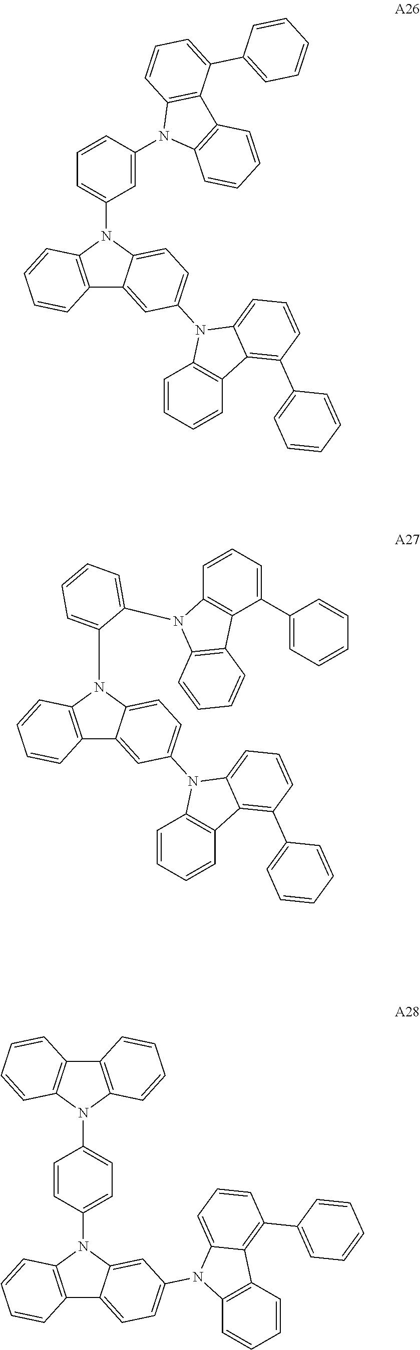 [SMILES: c1ccc(-c2cccc3c2c2ccccc2n3-c2cccc(-n3c4ccccc4c4cc(-n5c6ccccc6c6c(-c7ccccc7)cccc65)ccc43)c2)cc1 ; c1ccc(-c2cccc3c2c2ccccc2n3-c2ccc3c(c2)c2ccccc2n3-c2ccccc2-n2c3ccccc3c3c(-c4ccccc4)cccc32)cc1 ; c1ccc(-c2cccc3c2c2ccccc2n3-c2ccc3c4ccccc4n(-c4ccc(-n5c6ccccc6c6ccccc65)cc4)c3c2)cc1]

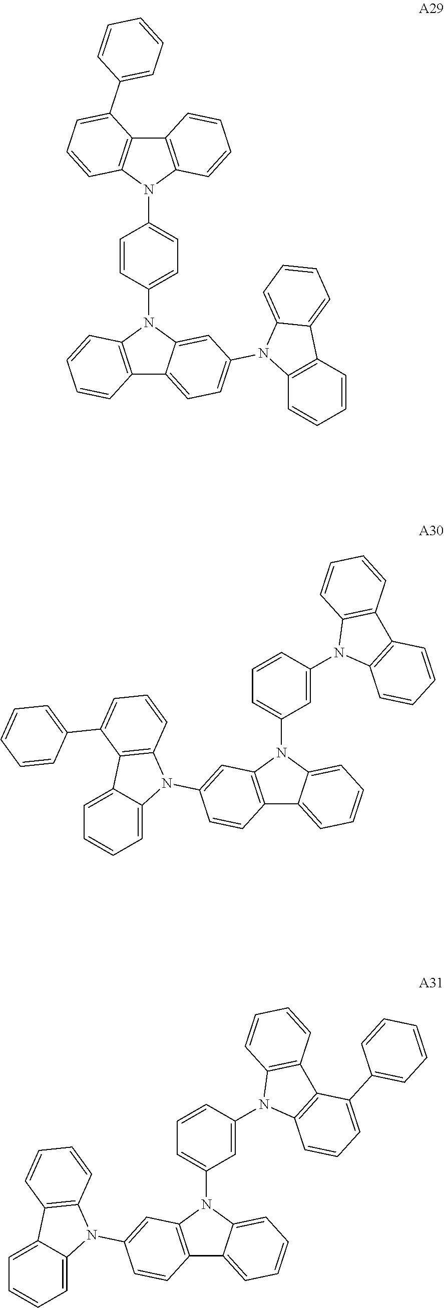 [SMILES: c1ccc(-c2cccc3c2c2ccccc2n3-c2ccc(-n3c4ccccc4c4ccc(-n5c6ccccc6c6ccccc65)cc43)cc2)cc1 ; c1ccc(-c2cccc3c2c2ccccc2n3-c2ccc3c4ccccc4n(-c4cccc(-n5c6ccccc6c6ccccc65)c4)c3c2)cc1 ; c1ccc(-c2cccc3c2c2ccccc2n3-c2cccc(-n3c4ccccc4c4ccc(-n5c6ccccc6c6ccccc65)cc43)c2)cc1]

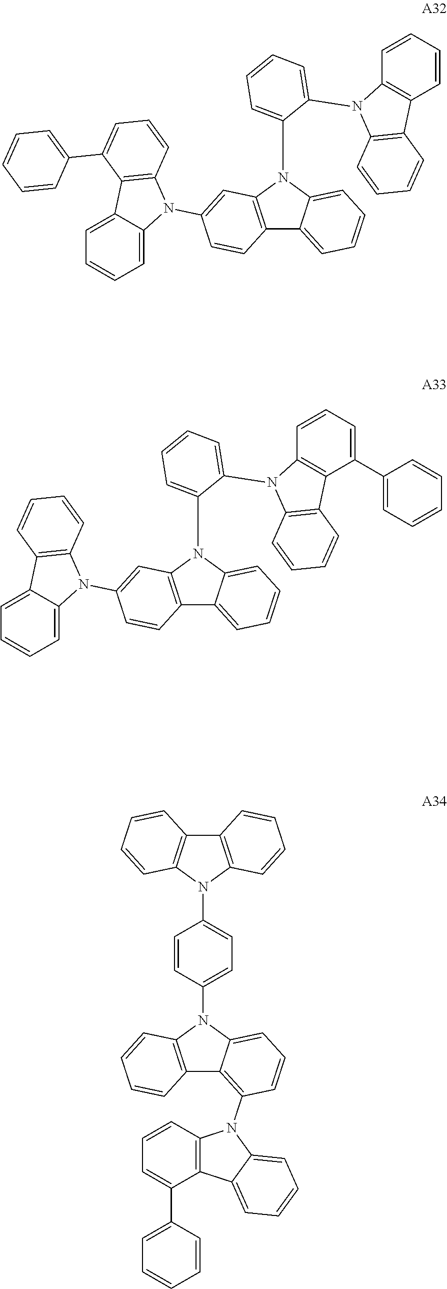 [SMILES: c1ccc(-c2cccc3c2c2ccccc2n3-c2ccc3c4ccccc4n(-c4ccccc4-n4c5ccccc5c5ccccc54)c3c2)cc1 ; c1ccc(-c2cccc3c2c2ccccc2n3-c2ccccc2-n2c3ccccc3c3ccc(-n4c5ccccc5c5ccccc54)cc32)cc1 ; c1ccc(-c2cccc3c2c2ccccc2n3-c2cccc3c2c2ccccc2n3-c2ccc(-n3c4ccccc4c4ccccc43)cc2)cc1]

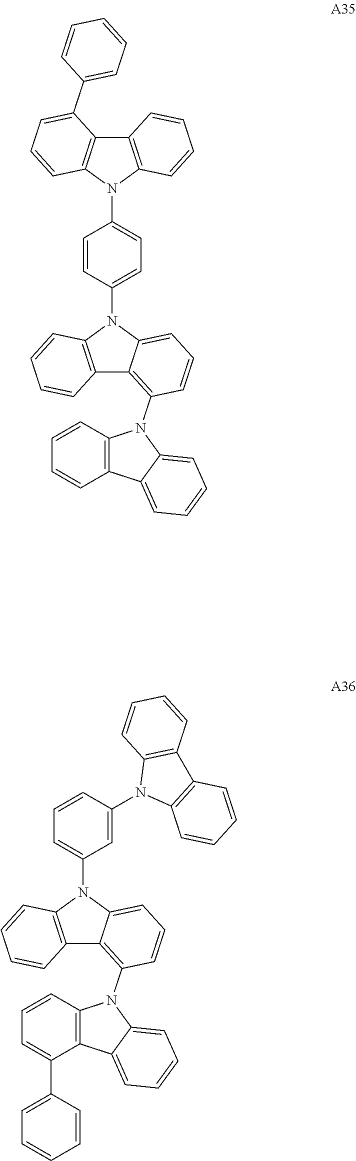 [SMILES: c1ccc(-c2cccc3c2c2ccccc2n3-c2ccc(-n3c4ccccc4c4c(-n5c6ccccc6c6ccccc65)cccc43)cc2)cc1 ; c1ccc(-c2cccc3c2c2ccccc2n3-c2cccc3c2c2ccccc2n3-c2cccc(-n3c4ccccc4c4ccccc43)c2)cc1]

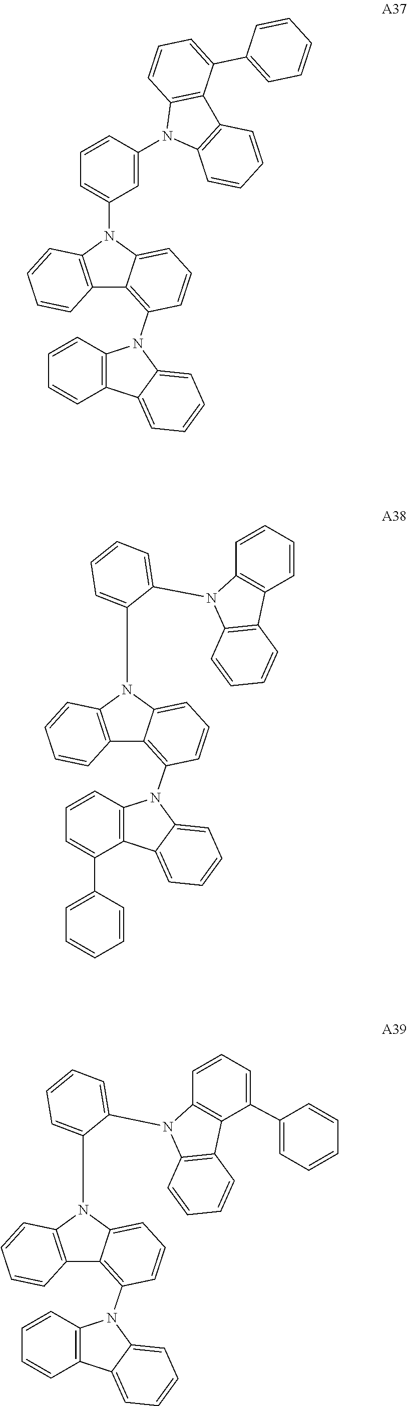 [SMILES: c1ccc(-c2cccc3c2c2ccccc2n3-c2cccc(-n3c4ccccc4c4c(-n5c6ccccc6c6ccccc65)cccc43)c2)cc1 ; c1ccc(-c2cccc3c2c2ccccc2n3-c2cccc3c2c2ccccc2n3-c2ccccc2-n2c3ccccc3c3ccccc32)cc1 ; c1ccc(-c2cccc3c2c2ccccc2n3-c2ccccc2-n2c3ccccc3c3c(-n4c5ccccc5c5ccccc54)cccc32)cc1]

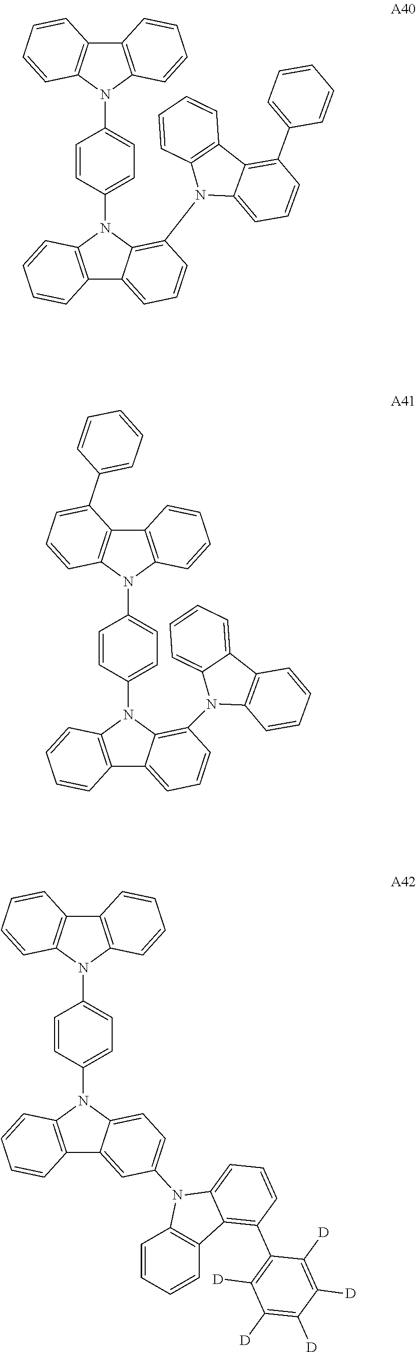 [SMILES: c1ccc(-c2cccc3c2c2ccccc2n3-c2cccc3c4ccccc4n(-c4ccc(-n5c6ccccc6c6ccccc65)cc4)c23)cc1 ; c1ccc(-c2cccc3c2c2ccccc2n3-c2ccc(-n3c4ccccc4c4cccc(-n5c6ccccc6c6ccccc65)c43)cc2)cc1 ; [2H]c1c([2H])c([2H])c(-c2cccc3c2c2ccccc2n3-c2ccc3c(c2)c2ccccc2n3-c2ccc(-n3c4ccccc4c4ccccc43)cc2)c([2H])c1[2H]]

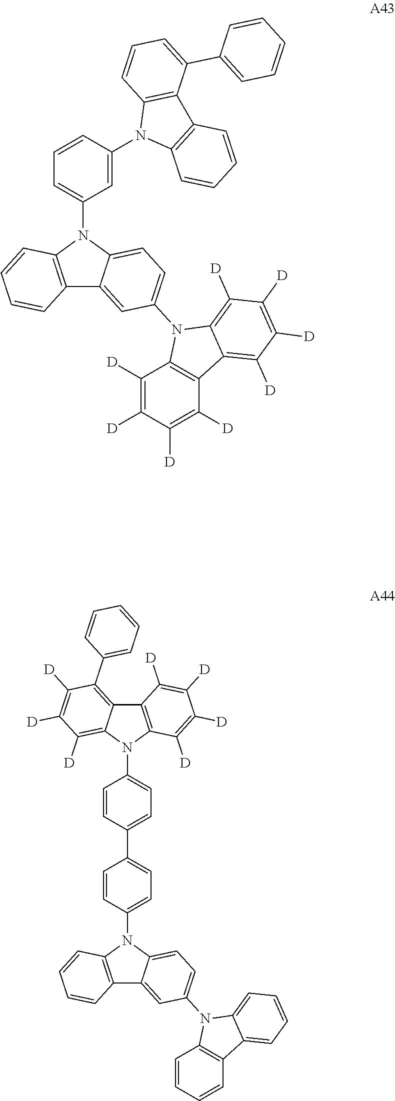 [SMILES: [2H]c1c([2H])c([2H])c2c(c1[2H])c1c([2H])c([2H])c([2H])c([2H])c1n2-c1ccc2c(c1)c1ccccc1n2-c1cccc(-n2c3ccccc3c3c(-c4ccccc4)cccc32)c1 ; [2H]c1c([2H])c([2H])c2c(c1[2H])c1c(-c3ccccc3)c([2H])c([2H])c([2H])c1n2-c1ccc(-c2ccc(-n3c4ccccc4c4cc(-n5c6ccccc6c6ccccc65)ccc43)cc2)cc1]

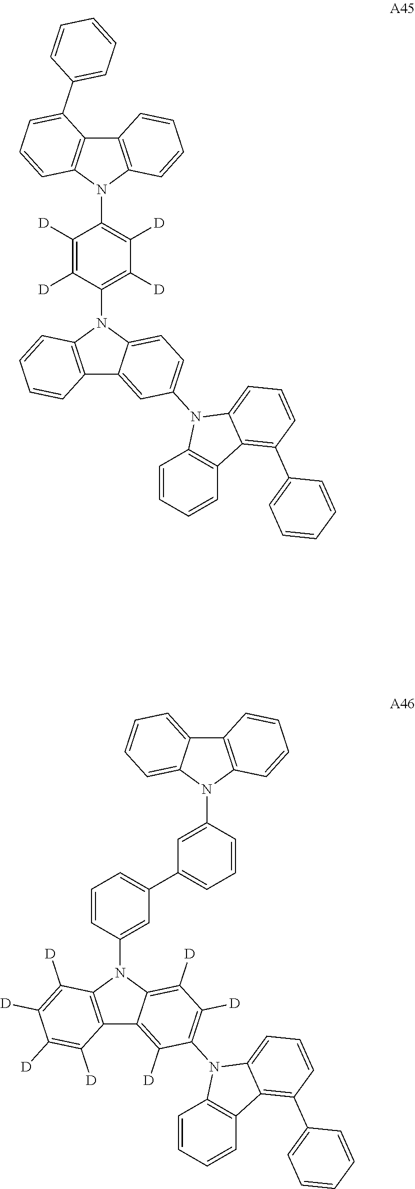 [SMILES: [2H]c1c([2H])c(-n2c3ccccc3c3c(-c4ccccc4)cccc32)c([2H])c([2H])c1-n1c2ccccc2c2cc(-n3c4ccccc4c4c(-c5ccccc5)cccc43)ccc21 ; [2H]c1c([2H])c([2H])c2c(c1[2H])c1c([2H])c(-n3c4ccccc4c4c(-c5ccccc5)cccc43)c([2H])c([2H])c1n2-c1cccc(-c2cccc(-n3c4ccccc4c4ccccc43)c2)c1]

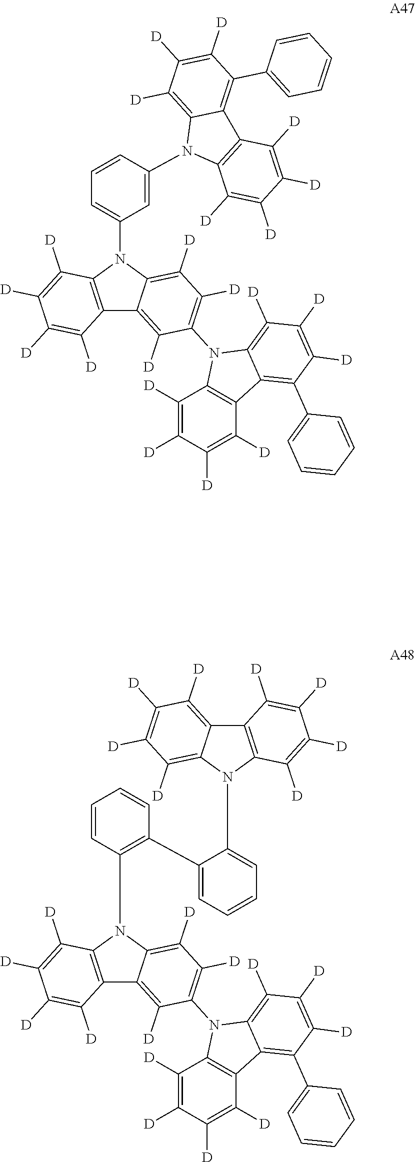 [SMILES: [2H]c1c([2H])c([2H])c2c(c1[2H])c1c([2H])c(-n3c4c([2H])c([2H])c([2H])c([2H])c4c4c(-c5ccccc5)c([2H])c([2H])c([2H])c43)c([2H])c([2H])c1n2-c1cccc(-n2c3c([2H])c([2H])c([2H])c([2H])c3c3c(-c4ccccc4)c([2H])c([2H])c([2H])c32)c1 ; [2H]c1c([2H])c([2H])c2c(c1[2H])c1c([2H])c([2H])c([2H])c([2H])c1n2-c1ccccc1-c1ccccc1-n1c2c([2H])c([2H])c([2H])c([2H])c2c2c([2H])c(-n3c4c([2H])c([2H])c([2H])c([2H])c4c4c(-c5ccccc5)c([2H])c([2H])c([2H])c43)c([2H])c([2H])c21]

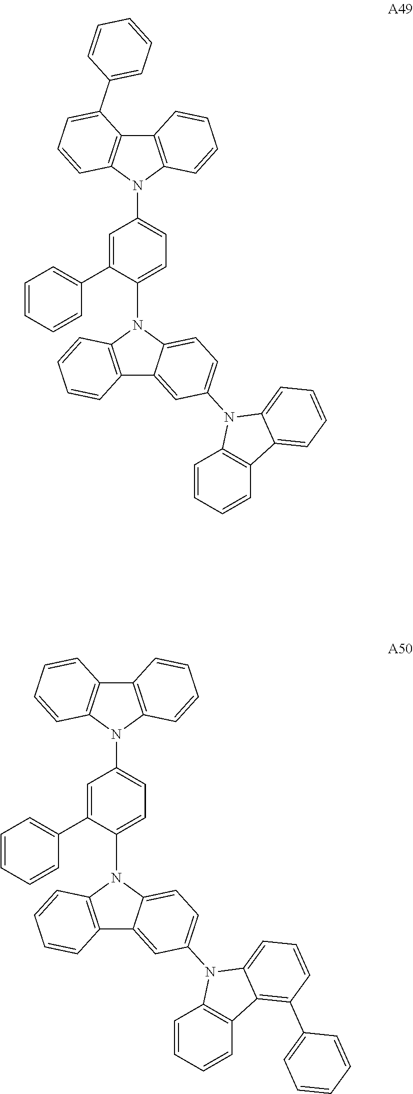 [SMILES: c1ccc(-c2cc(-n3c4ccccc4c4c(-c5ccccc5)cccc43)ccc2-n2c3ccccc3c3cc(-n4c5ccccc5c5ccccc54)ccc32)cc1 ; c1ccc(-c2cc(-n3c4ccccc4c4ccccc43)ccc2-n2c3ccccc3c3cc(-n4c5ccccc5c5c(-c6ccccc6)cccc54)ccc32)cc1]

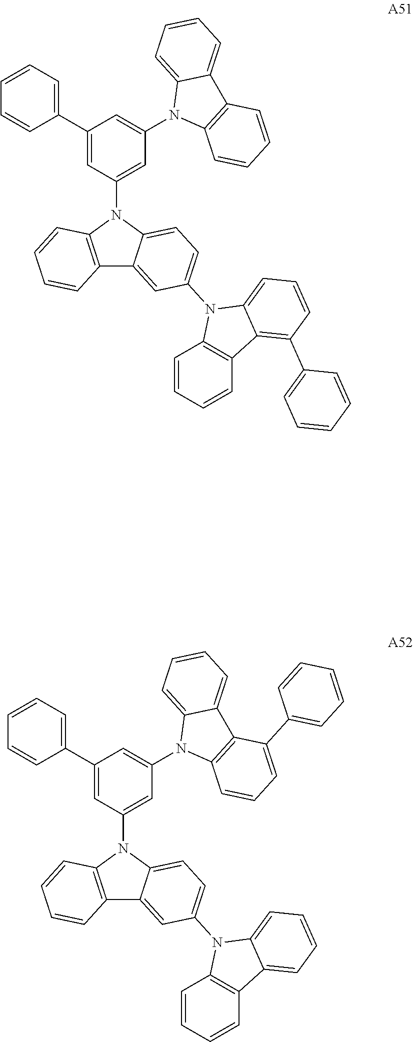 [SMILES: c1ccc(-c2cc(-n3c4ccccc4c4ccccc43)cc(-n3c4ccccc4c4cc(-n5c6ccccc6c6c(-c7ccccc7)cccc65)ccc43)c2)cc1 ; c1ccc(-c2cc(-n3c4ccccc4c4cc(-n5c6ccccc6c6ccccc65)ccc43)cc(-n3c4ccccc4c4c(-c5ccccc5)cccc43)c2)cc1]

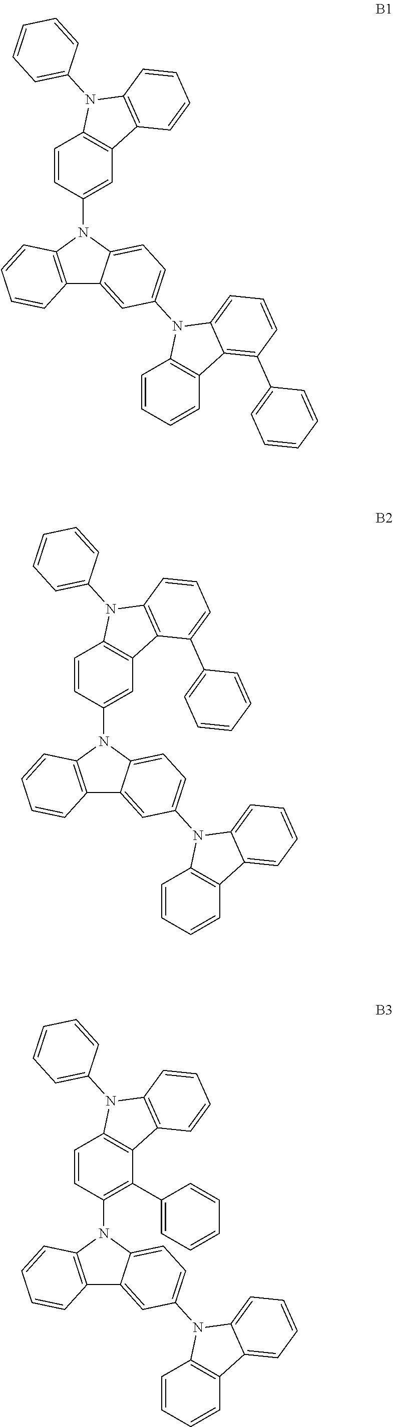 [SMILES: c1ccc(-c2cccc3c2c2ccccc2n3-c2ccc3c(c2)c2ccccc2n3-c2ccc3c(c2)c2ccccc2n3-c2ccccc2)cc1 ; c1ccc(-c2cccc3c2c2cc(-n4c5ccccc5c5cc(-n6c7ccccc7c7ccccc76)ccc54)ccc2n3-c2ccccc2)cc1 ; c1ccc(-c2c(-n3c4ccccc4c4cc(-n5c6ccccc6c6ccccc65)ccc43)ccc3c2c2ccccc2n3-c2ccccc2)cc1]

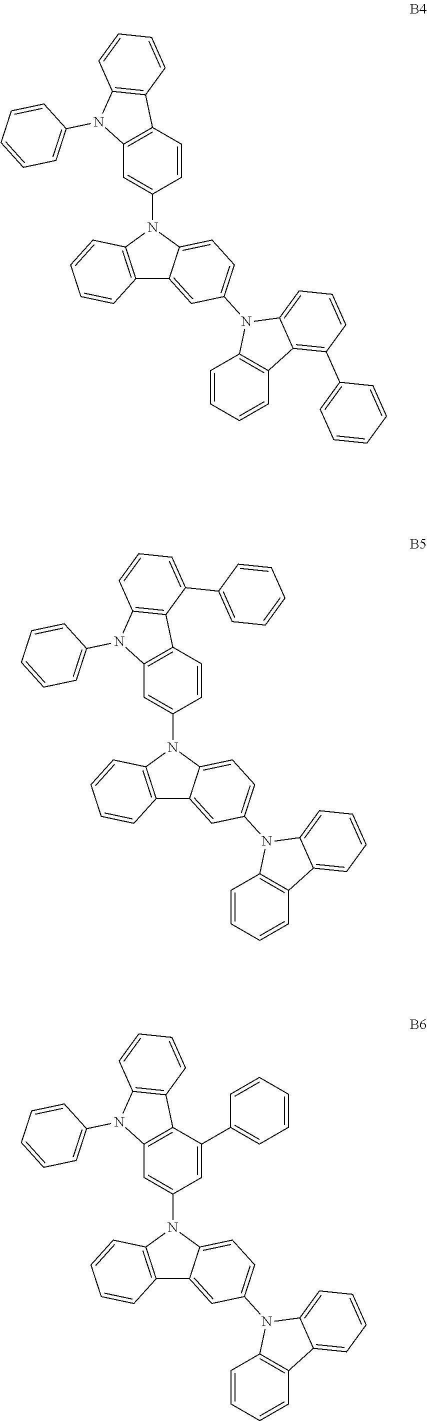 [SMILES: c1ccc(-c2cccc3c2c2ccccc2n3-c2ccc3c(c2)c2ccccc2n3-c2ccc3c4ccccc4n(-c4ccccc4)c3c2)cc1 ; c1ccc(-c2cccc3c2c2ccc(-n4c5ccccc5c5cc(-n6c7ccccc7c7ccccc76)ccc54)cc2n3-c2ccccc2)cc1 ; c1ccc(-c2cc(-n3c4ccccc4c4cc(-n5c6ccccc6c6ccccc65)ccc43)cc3c2c2ccccc2n3-c2ccccc2)cc1]

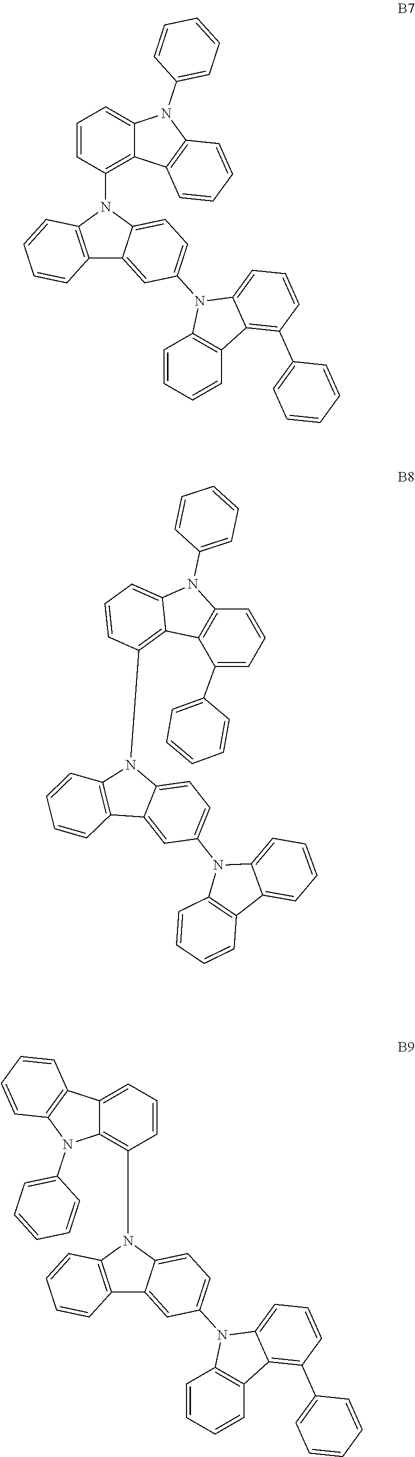 [SMILES: c1ccc(-c2cccc3c2c2ccccc2n3-c2ccc3c(c2)c2ccccc2n3-c2cccc3c2c2ccccc2n3-c2ccccc2)cc1 ; c1ccc(-c2cccc3c2c2c(-n4c5ccccc5c5cc(-n6c7ccccc7c7ccccc76)ccc54)cccc2n3-c2ccccc2)cc1 ; c1ccc(-c2cccc3c2c2ccccc2n3-c2ccc3c(c2)c2ccccc2n3-c2cccc3c4ccccc4n(-c4ccccc4)c23)cc1]

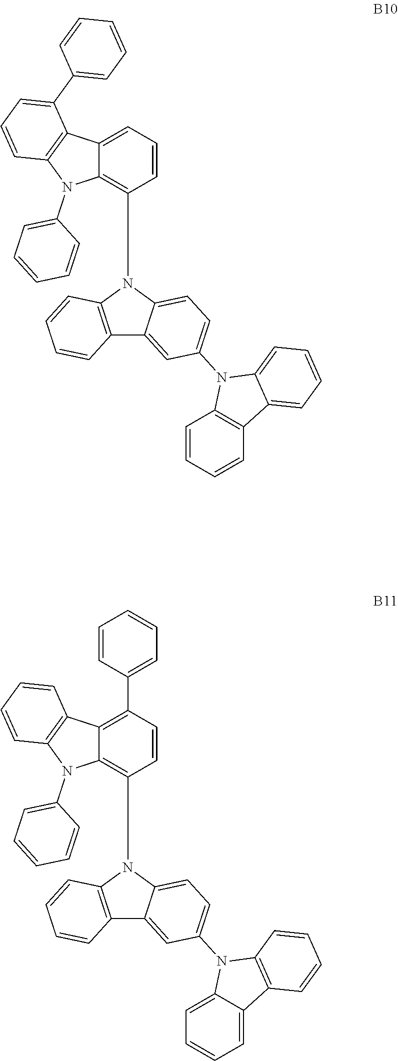 [SMILES: c1ccc(-c2cccc3c2c2cccc(-n4c5ccccc5c5cc(-n6c7ccccc7c7ccccc76)ccc54)c2n3-c2ccccc2)cc1 ; c1ccc(-c2ccc(-n3c4ccccc4c4cc(-n5c6ccccc6c6ccccc65)ccc43)c3c2c2ccccc2n3-c2ccccc2)cc1]

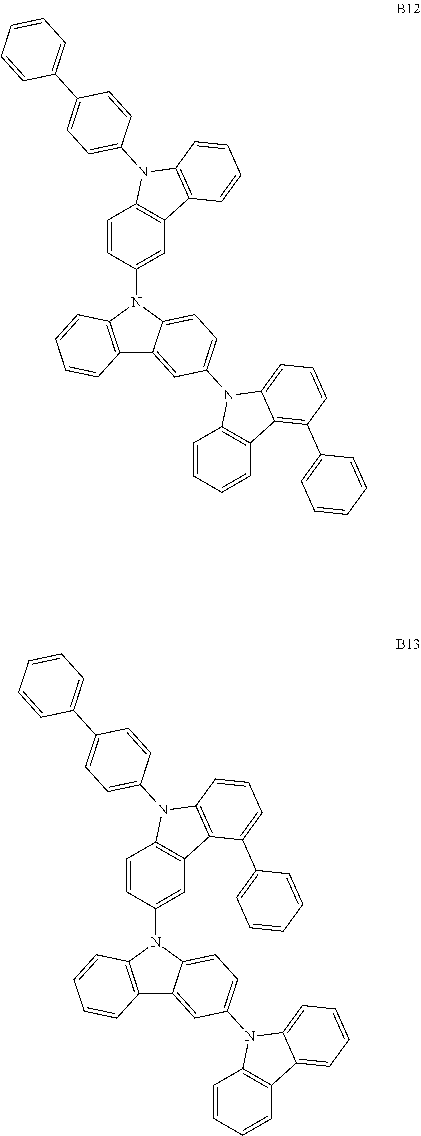 [SMILES: c1ccc(-c2ccc(-n3c4ccccc4c4cc(-n5c6ccccc6c6cc(-n7c8ccccc8c8c(-c9ccccc9)cccc87)ccc65)ccc43)cc2)cc1 ; c1ccc(-c2ccc(-n3c4ccc(-n5c6ccccc6c6cc(-n7c8ccccc8c8ccccc87)ccc65)cc4c4c(-c5ccccc5)cccc43)cc2)cc1]

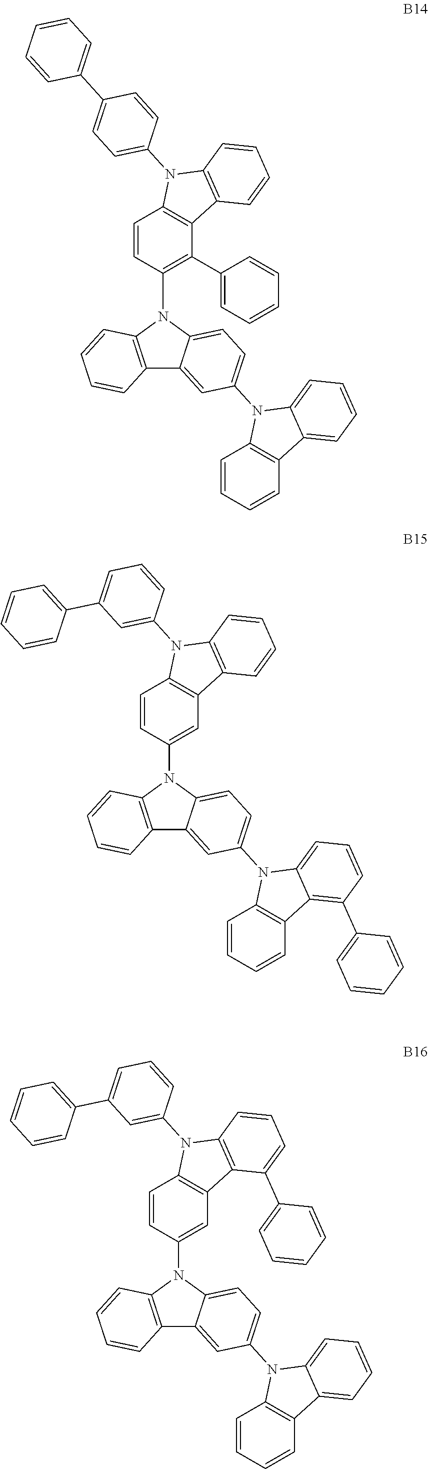 [SMILES: c1ccc(-c2ccc(-n3c4ccccc4c4c(-c5ccccc5)c(-n5c6ccccc6c6cc(-n7c8ccccc8c8ccccc87)ccc65)ccc43)cc2)cc1 ; c1ccc(-c2cccc(-n3c4ccccc4c4cc(-n5c6ccccc6c6cc(-n7c8ccccc8c8c(-c9ccccc9)cccc87)ccc65)ccc43)c2)cc1 ; c1ccc(-c2cccc(-n3c4ccc(-n5c6ccccc6c6cc(-n7c8ccccc8c8ccccc87)ccc65)cc4c4c(-c5ccccc5)cccc43)c2)cc1]

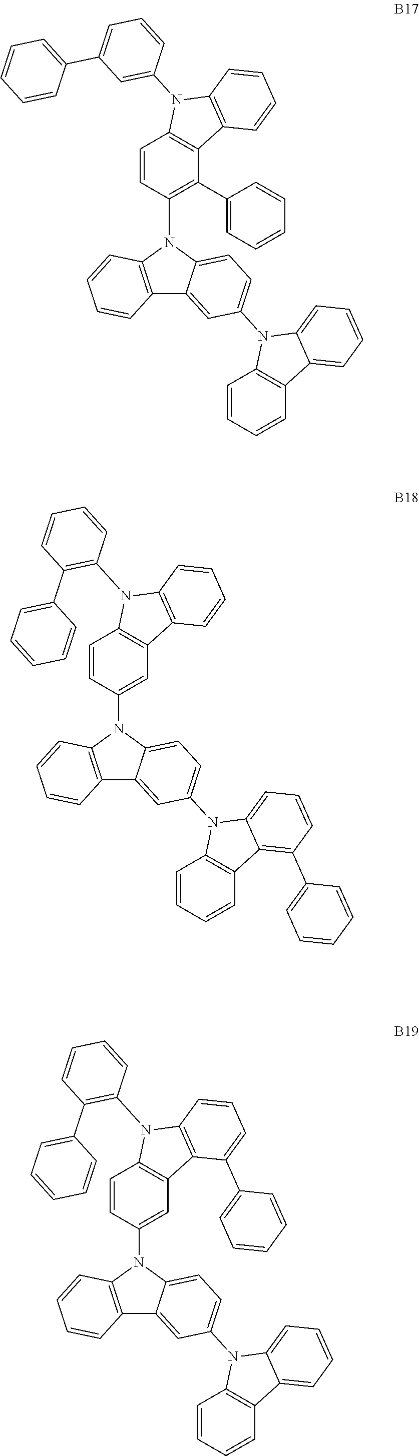 [SMILES: c1ccc(-c2cccc(-n3c4ccccc4c4c(-c5ccccc5)c(-n5c6ccccc6c6cc(-n7c8ccccc8c8ccccc87)ccc65)ccc43)c2)cc1 ; c1ccc(-c2ccccc2-n2c3ccccc3c3cc(-n4c5ccccc5c5cc(-n6c7ccccc7c7c(-c8ccccc8)cccc76)ccc54)ccc32)cc1 ; c1ccc(-c2ccccc2-n2c3ccc(-n4c5ccccc5c5cc(-n6c7ccccc7c7ccccc76)ccc54)cc3c3c(-c4ccccc4)cccc32)cc1]

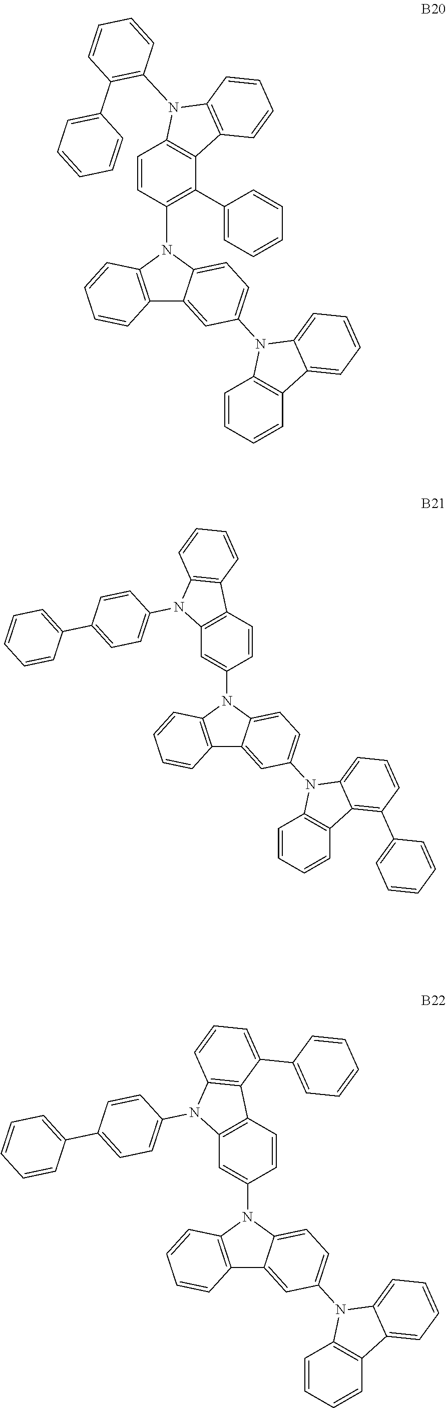 [SMILES: c1ccc(-c2ccccc2-n2c3ccccc3c3c(-c4ccccc4)c(-n4c5ccccc5c5cc(-n6c7ccccc7c7ccccc76)ccc54)ccc32)cc1 ; c1ccc(-c2ccc(-n3c4ccccc4c4ccc(-n5c6ccccc6c6cc(-n7c8ccccc8c8c(-c9ccccc9)cccc87)ccc65)cc43)cc2)cc1 ; c1ccc(-c2ccc(-n3c4cc(-n5c6ccccc6c6cc(-n7c8ccccc8c8ccccc87)ccc65)ccc4c4c(-c5ccccc5)cccc43)cc2)cc1]

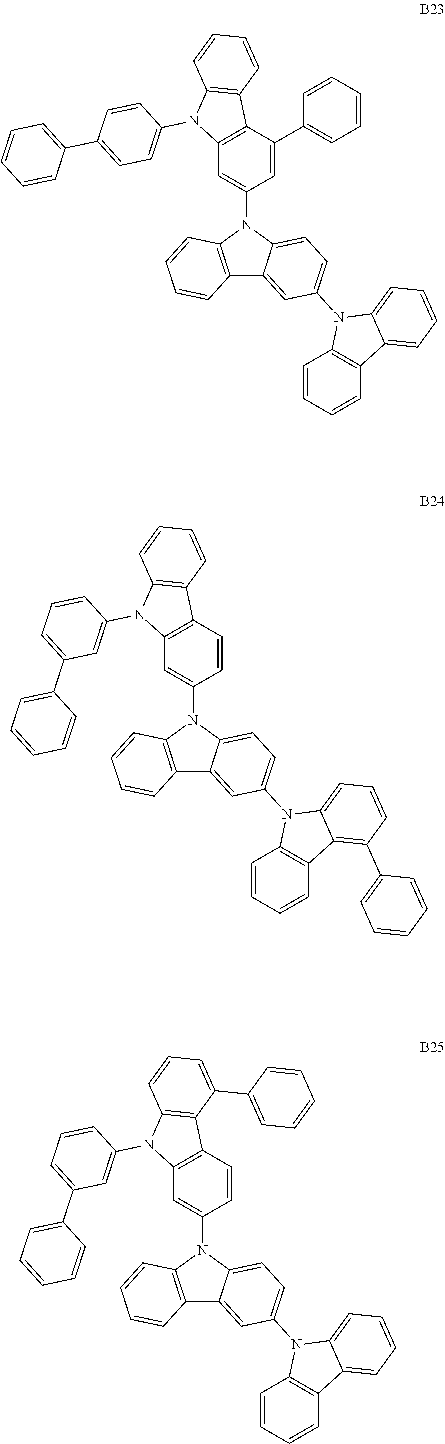 [SMILES: c1ccc(-c2ccc(-n3c4ccccc4c4c(-c5ccccc5)cc(-n5c6ccccc6c6cc(-n7c8ccccc8c8ccccc87)ccc65)cc43)cc2)cc1 ; c1ccc(-c2cccc(-n3c4ccccc4c4ccc(-n5c6ccccc6c6cc(-n7c8ccccc8c8c(-c9ccccc9)cccc87)ccc65)cc43)c2)cc1 ; c1ccc(-c2cccc(-n3c4cc(-n5c6ccccc6c6cc(-n7c8ccccc8c8ccccc87)ccc65)ccc4c4c(-c5ccccc5)cccc43)c2)cc1]

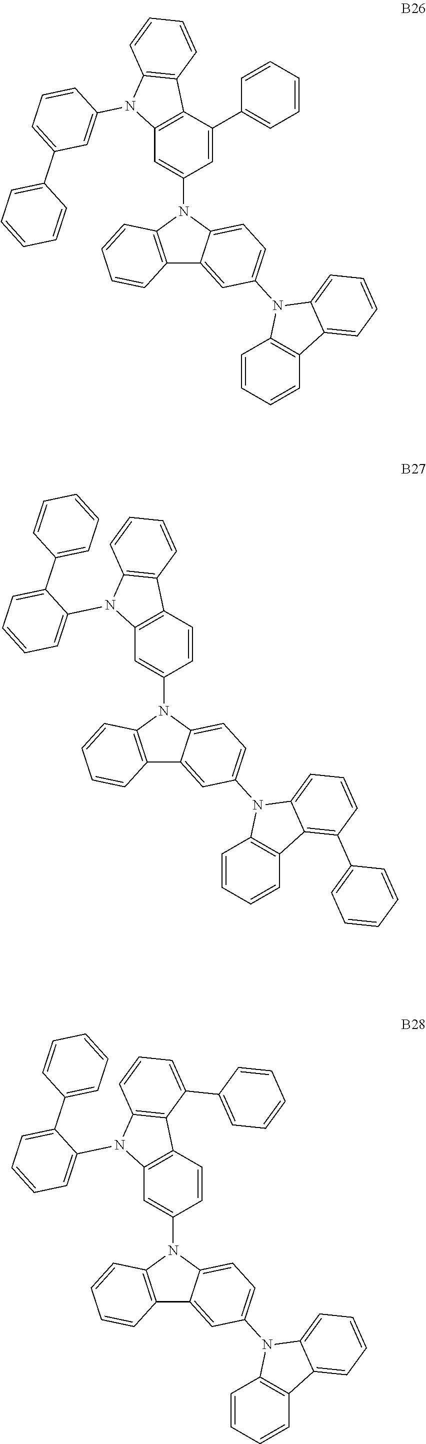 [SMILES: c1ccc(-c2cccc(-n3c4ccccc4c4c(-c5ccccc5)cc(-n5c6ccccc6c6cc(-n7c8ccccc8c8ccccc87)ccc65)cc43)c2)cc1 ; c1ccc(-c2ccccc2-n2c3ccccc3c3ccc(-n4c5ccccc5c5cc(-n6c7ccccc7c7c(-c8ccccc8)cccc76)ccc54)cc32)cc1 ; c1ccc(-c2ccccc2-n2c3cc(-n4c5ccccc5c5cc(-n6c7ccccc7c7ccccc76)ccc54)ccc3c3c(-c4ccccc4)cccc32)cc1]

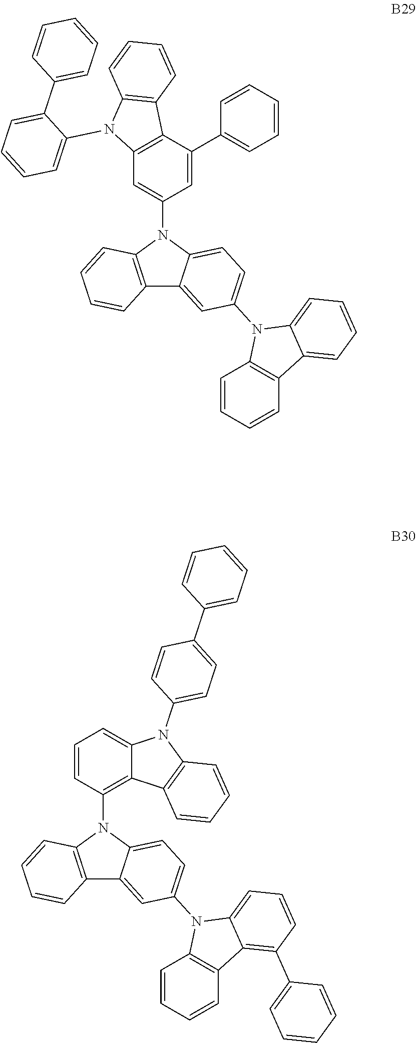 [SMILES: c1ccc(-c2ccccc2-n2c3ccccc3c3c(-c4ccccc4)cc(-n4c5ccccc5c5cc(-n6c7ccccc7c7ccccc76)ccc54)cc32)cc1 ; c1ccc(-c2ccc(-n3c4ccccc4c4c(-n5c6ccccc6c6cc(-n7c8ccccc8c8c(-c9ccccc9)cccc87)ccc65)cccc43)cc2)cc1]

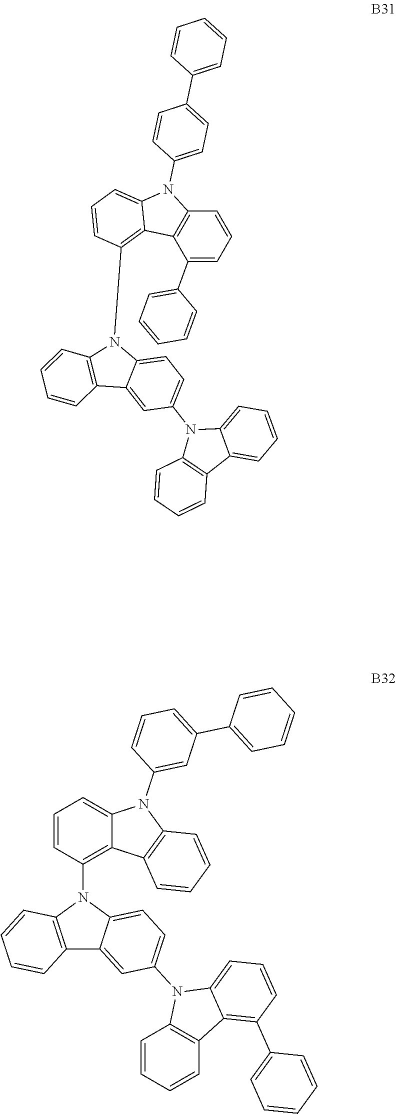 [SMILES: c1ccc(-c2ccc(-n3c4cccc(-c5ccccc5)c4c4c(-n5c6ccccc6c6cc(-n7c8ccccc8c8ccccc87)ccc65)cccc43)cc2)cc1 ; c1ccc(-c2cccc(-n3c4ccccc4c4c(-n5c6ccccc6c6cc(-n7c8ccccc8c8c(-c9ccccc9)cccc87)ccc65)cccc43)c2)cc1]

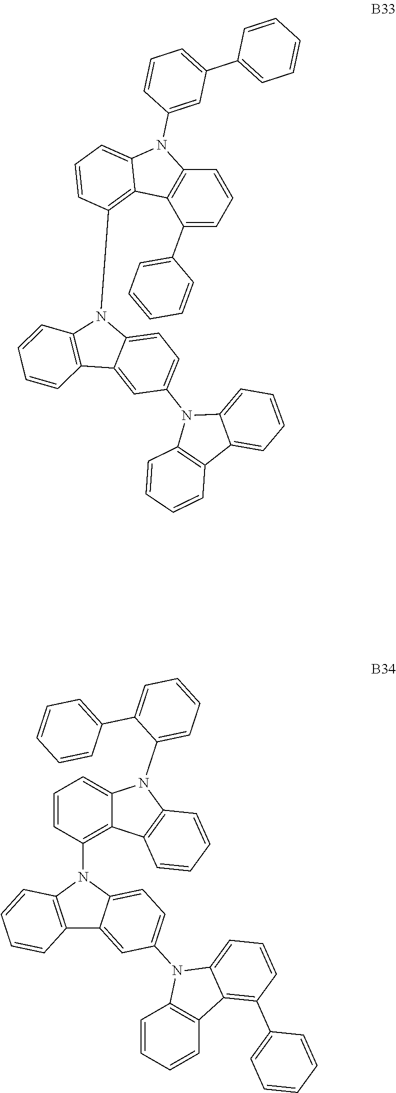 [SMILES: c1ccc(-c2cccc(-n3c4cccc(-c5ccccc5)c4c4c(-n5c6ccccc6c6cc(-n7c8ccccc8c8ccccc87)ccc65)cccc43)c2)cc1 ; c1ccc(-c2ccccc2-n2c3ccccc3c3c(-n4c5ccccc5c5cc(-n6c7ccccc7c7c(-c8ccccc8)cccc76)ccc54)cccc32)cc1]

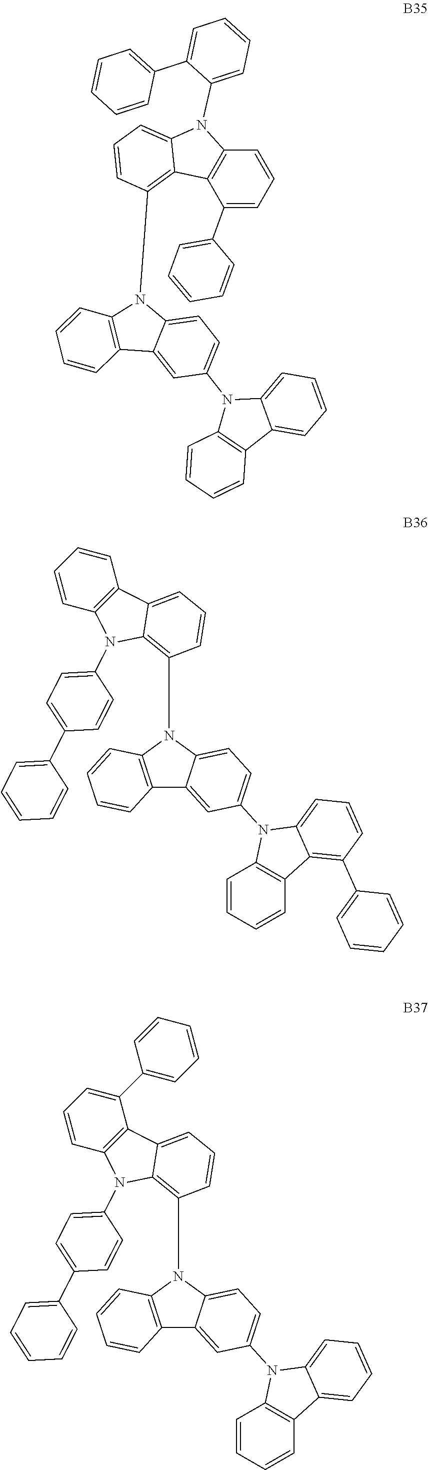 [SMILES: c1ccc(-c2ccccc2-n2c3cccc(-c4ccccc4)c3c3c(-n4c5ccccc5c5cc(-n6c7ccccc7c7ccccc76)ccc54)cccc32)cc1 ; c1ccc(-c2ccc(-n3c4ccccc4c4cccc(-n5c6ccccc6c6cc(-n7c8ccccc8c8c(-c9ccccc9)cccc87)ccc65)c43)cc2)cc1 ; c1ccc(-c2ccc(-n3c4cccc(-c5ccccc5)c4c4cccc(-n5c6ccccc6c6cc(-n7c8ccccc8c8ccccc87)ccc65)c43)cc2)cc1]

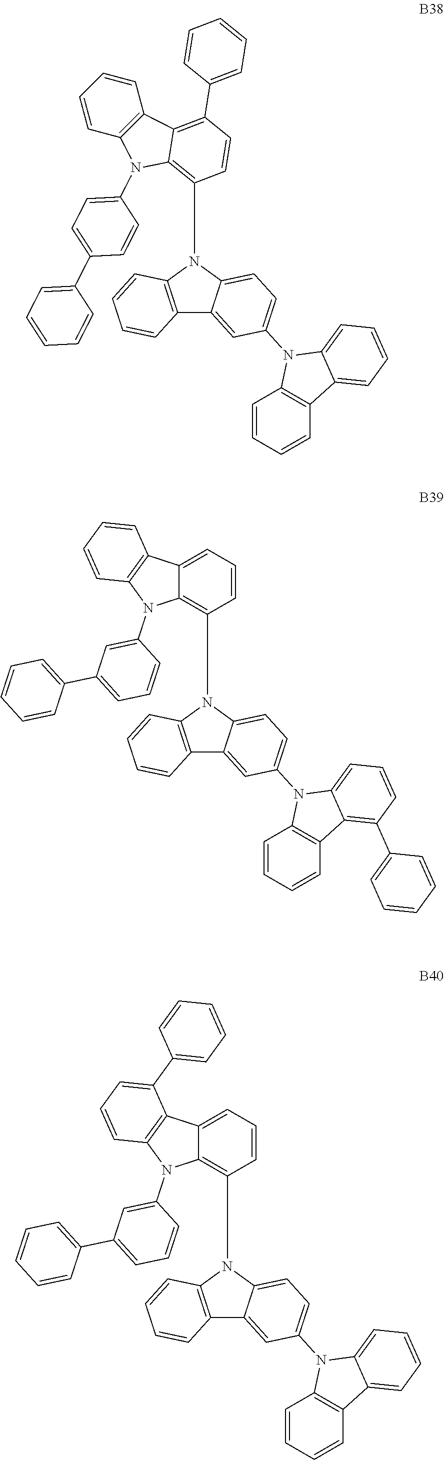 [SMILES: c1ccc(-c2ccc(-n3c4ccccc4c4c(-c5ccccc5)ccc(-n5c6ccccc6c6cc(-n7c8ccccc8c8ccccc87)ccc65)c43)cc2)cc1 ; c1ccc(-c2cccc(-n3c4ccccc4c4cccc(-n5c6ccccc6c6cc(-n7c8ccccc8c8c(-c9ccccc9)cccc87)ccc65)c43)c2)cc1 ; c1ccc(-c2cccc(-n3c4cccc(-c5ccccc5)c4c4cccc(-n5c6ccccc6c6cc(-n7c8ccccc8c8ccccc87)ccc65)c43)c2)cc1]

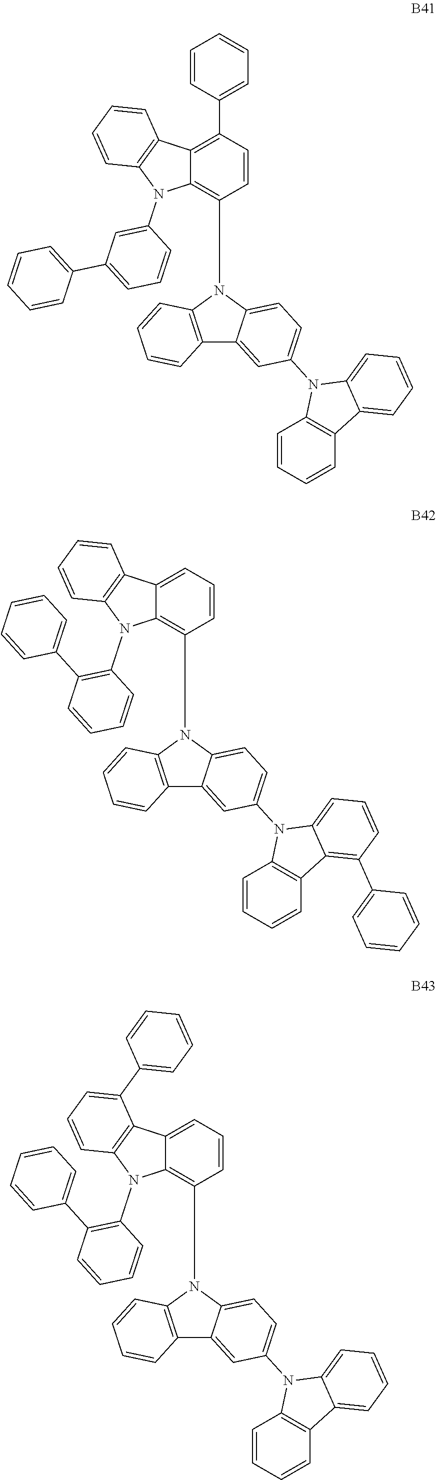 [SMILES: c1ccc(-c2cccc(-n3c4ccccc4c4c(-c5ccccc5)ccc(-n5c6ccccc6c6cc(-n7c8ccccc8c8ccccc87)ccc65)c43)c2)cc1 ; c1ccc(-c2ccccc2-n2c3ccccc3c3cccc(-n4c5ccccc5c5cc(-n6c7ccccc7c7c(-c8ccccc8)cccc76)ccc54)c32)cc1 ; c1ccc(-c2ccccc2-n2c3cccc(-c4ccccc4)c3c3cccc(-n4c5ccccc5c5cc(-n6c7ccccc7c7ccccc76)ccc54)c32)cc1]

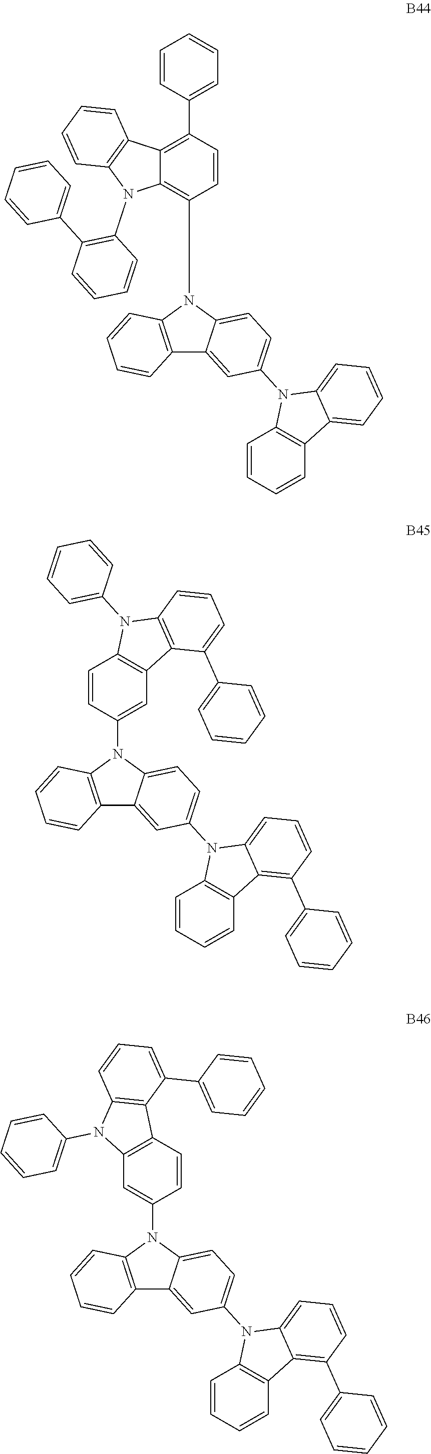 [SMILES: c1ccc(-c2ccccc2-n2c3ccccc3c3c(-c4ccccc4)ccc(-n4c5ccccc5c5cc(-n6c7ccccc7c7ccccc76)ccc54)c32)cc1 ; c1ccc(-c2cccc3c2c2ccccc2n3-c2ccc3c(c2)c2ccccc2n3-c2ccc3c(c2)c2c(-c4ccccc4)cccc2n3-c2ccccc2)cc1 ; c1ccc(-c2cccc3c2c2ccccc2n3-c2ccc3c(c2)c2ccccc2n3-c2ccc3c4c(-c5ccccc5)cccc4n(-c4ccccc4)c3c2)cc1]

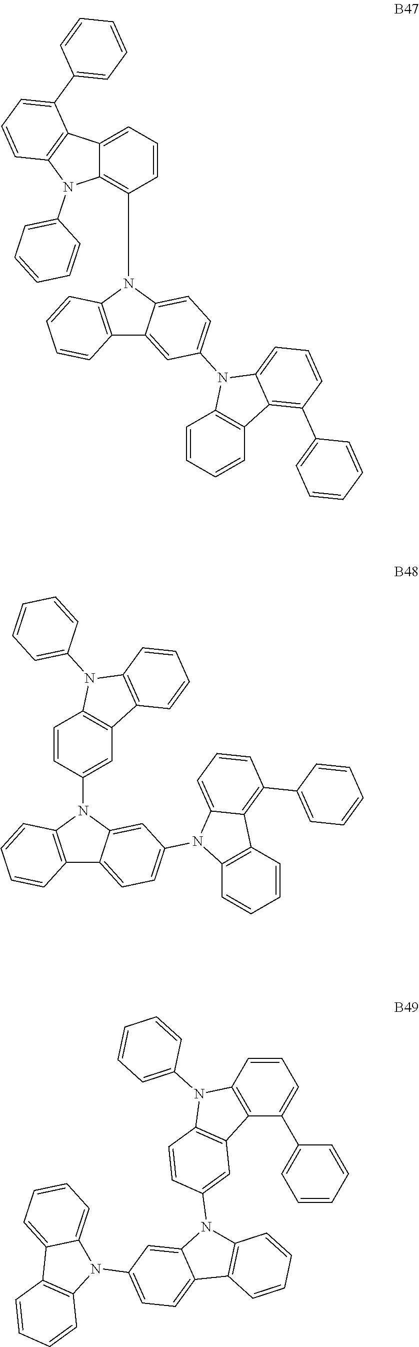 [SMILES: c1ccc(-c2cccc3c2c2ccccc2n3-c2ccc3c(c2)c2ccccc2n3-c2cccc3c4c(-c5ccccc5)cccc4n(-c4ccccc4)c23)cc1 ; c1ccc(-c2cccc3c2c2ccccc2n3-c2ccc3c4ccccc4n(-c4ccc5c(c4)c4ccccc4n5-c4ccccc4)c3c2)cc1 ; c1ccc(-c2cccc3c2c2cc(-n4c5ccccc5c5ccc(-n6c7ccccc7c7ccccc76)cc54)ccc2n3-c2ccccc2)cc1]

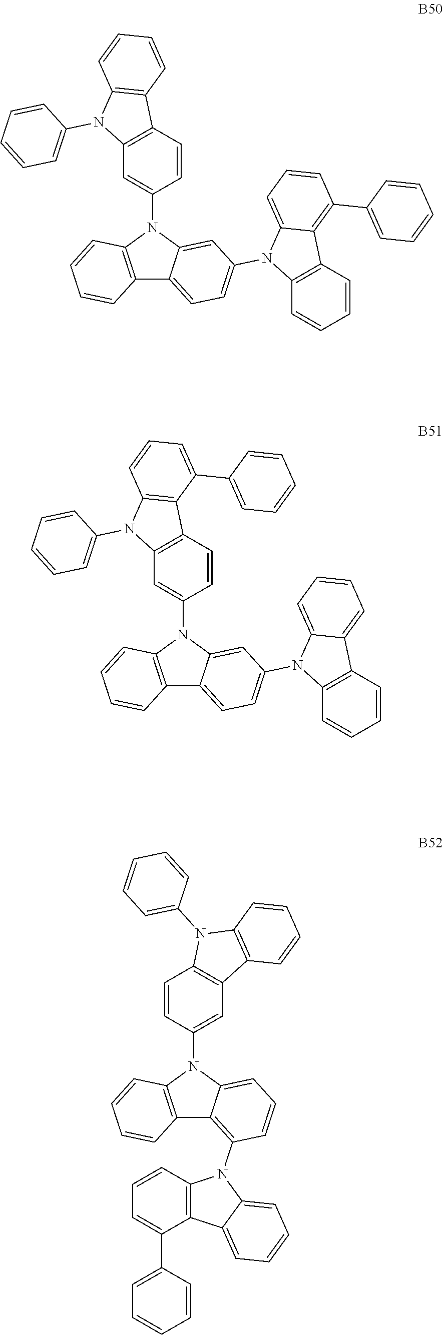 [SMILES: c1ccc(-c2cccc3c2c2ccccc2n3-c2ccc3c4ccccc4n(-c4ccc5c6ccccc6n(-c6ccccc6)c5c4)c3c2)cc1 ; c1ccc(-c2cccc3c2c2ccc(-n4c5ccccc5c5ccc(-n6c7ccccc7c7ccccc76)cc54)cc2n3-c2ccccc2)cc1 ; c1ccc(-c2cccc3c2c2ccccc2n3-c2cccc3c2c2ccccc2n3-c2ccc3c(c2)c2ccccc2n3-c2ccccc2)cc1]

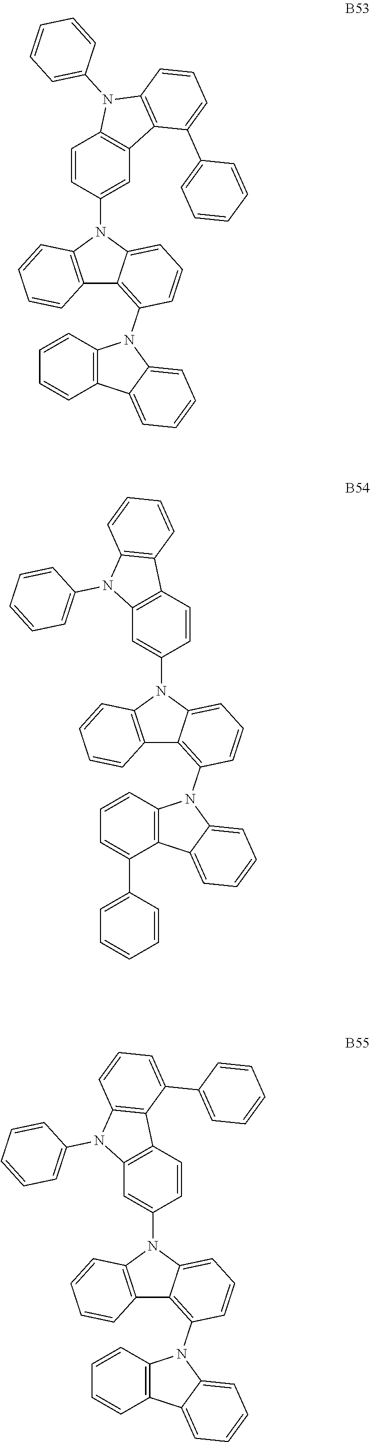 [SMILES: c1ccc(-c2cccc3c2c2cc(-n4c5ccccc5c5c(-n6c7ccccc7c7ccccc76)cccc54)ccc2n3-c2ccccc2)cc1 ; c1ccc(-c2cccc3c2c2ccccc2n3-c2cccc3c2c2ccccc2n3-c2ccc3c4ccccc4n(-c4ccccc4)c3c2)cc1 ; c1ccc(-c2cccc3c2c2ccc(-n4c5ccccc5c5c(-n6c7ccccc7c7ccccc76)cccc54)cc2n3-c2ccccc2)cc1]

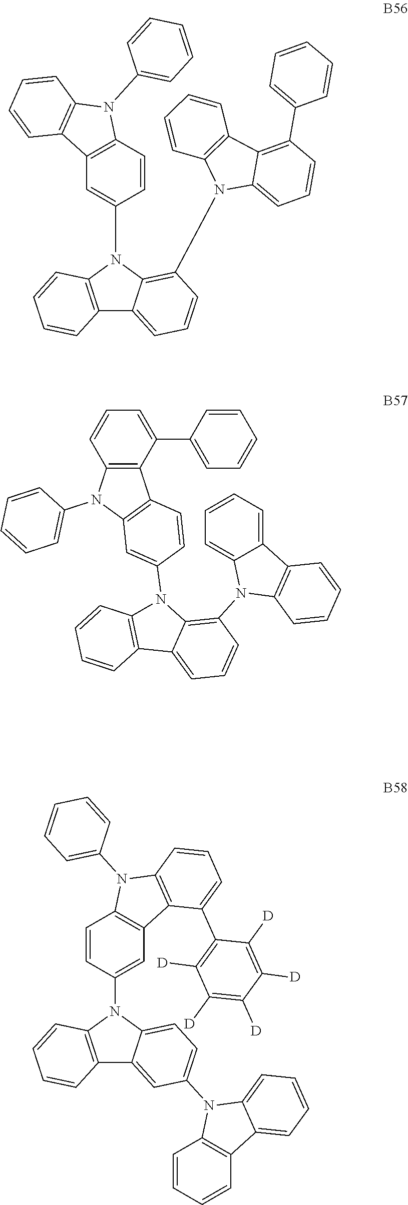 [SMILES: c1ccc(-c2cccc3c2c2ccccc2n3-c2cccc3c4ccccc4n(-c4ccc5c(c4)c4ccccc4n5-c4ccccc4)c23)cc1 ; c1ccc(-c2cccc3c2c2ccc(-n4c5ccccc5c5cccc(-n6c7ccccc7c7ccccc76)c54)cc2n3-c2ccccc2)cc1 ; [2H]c1c([2H])c([2H])c(-c2cccc3c2c2cc(-n4c5ccccc5c5cc(-n6c7ccccc7c7ccccc76)ccc54)ccc2n3-c2ccccc2)c([2H])c1[2H]]

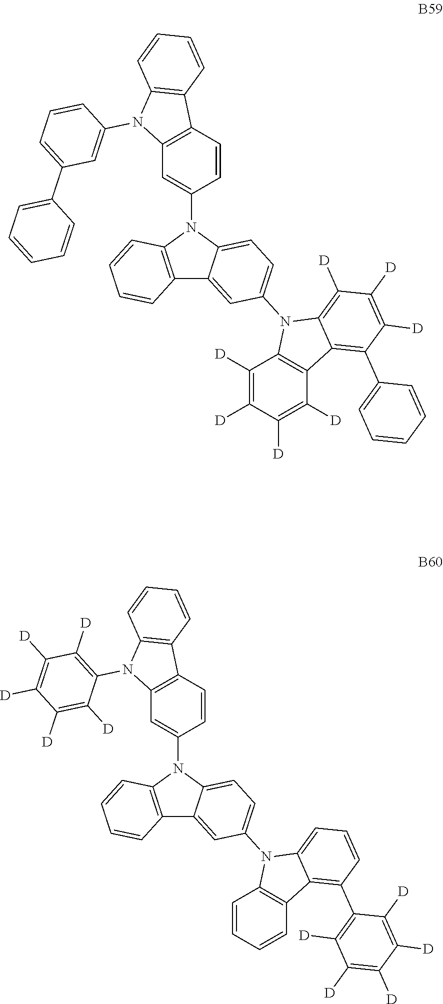 [SMILES: [2H]c1c([2H])c([2H])c2c(c1[2H])c1c(-c3ccccc3)c([2H])c([2H])c([2H])c1n2-c1ccc2c(c1)c1ccccc1n2-c1ccc2c3ccccc3n(-c3cccc(-c4ccccc4)c3)c2c1 ; [2H]c1c([2H])c([2H])c(-c2cccc3c2c2ccccc2n3-c2ccc3c(c2)c2ccccc2n3-c2ccc3c4ccccc4n(-c4c([2H])c([2H])c([2H])c([2H])c4[2H])c3c2)c([2H])c1[2H]]

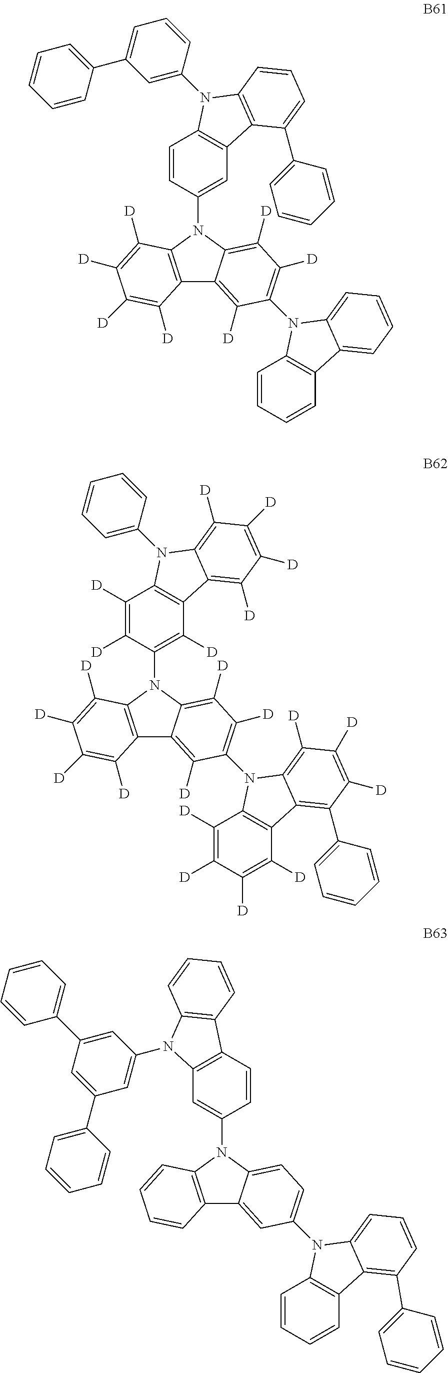 [SMILES: [2H]c1c([2H])c([2H])c2c(c1[2H])c1c([2H])c(-n3c4ccccc4c4ccccc43)c([2H])c([2H])c1n2-c1ccc2c(c1)c1c(-c3ccccc3)cccc1n2-c1cccc(-c2ccccc2)c1 ; [2H]c1c([2H])c([2H])c2c(c1[2H])c1c([2H])c(-n3c4c([2H])c([2H])c([2H])c([2H])c4c4c([2H])c(-n5c6c([2H])c([2H])c([2H])c([2H])c6c6c(-c7ccccc7)c([2H])c([2H])c([2H])c65)c([2H])c([2H])c43)c([2H])c([2H])c1n2-c1ccccc1 ; c1ccc(-c2cc(-c3ccccc3)cc(-n3c4ccccc4c4ccc(-n5c6ccccc6c6cc(-n7c8ccccc8c8c(-c9ccccc9)cccc87)ccc65)cc43)c2)cc1]

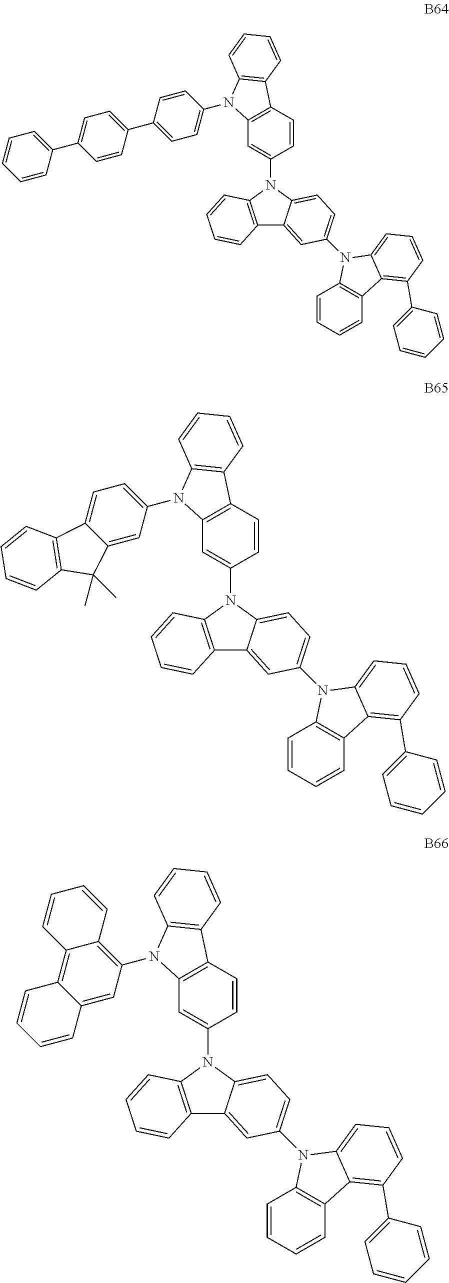 [SMILES: c1ccc(-c2ccc(-c3ccc(-n4c5ccccc5c5ccc(-n6c7ccccc7c7cc(-n8c9ccccc9c9c(-c%10ccccc%10)cccc98)ccc76)cc54)cc3)cc2)cc1 ; CC1(C)c2ccccc2-c2ccc(-n3c4ccccc4c4ccc(-n5c6ccccc6c6cc(-n7c8ccccc8c8c(-c9ccccc9)cccc87)ccc65)cc43)cc21 ; c1ccc(-c2cccc3c2c2ccccc2n3-c2ccc3c(c2)c2ccccc2n3-c2ccc3c4ccccc4n(-c4cc5ccccc5c5ccccc45)c3c2)cc1]

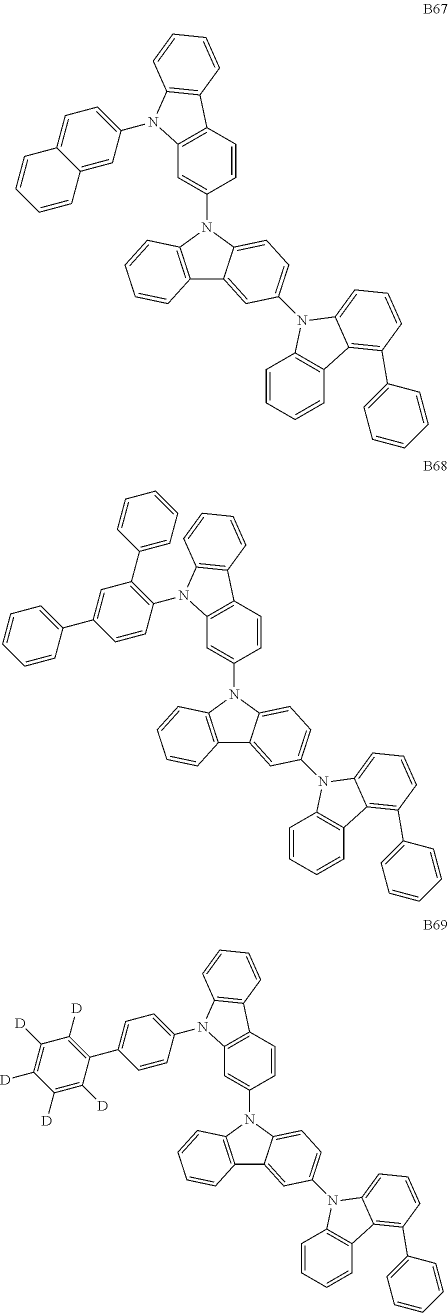 [SMILES: c1ccc(-c2cccc3c2c2ccccc2n3-c2ccc3c(c2)c2ccccc2n3-c2ccc3c4ccccc4n(-c4ccc5ccccc5c4)c3c2)cc1 ; c1ccc(-c2ccc(-n3c4ccccc4c4ccc(-n5c6ccccc6c6cc(-n7c8ccccc8c8c(-c9ccccc9)cccc87)ccc65)cc43)c(-c3ccccc3)c2)cc1 ; [2H]c1c([2H])c([2H])c(-c2ccc(-n3c4ccccc4c4ccc(-n5c6ccccc6c6cc(-n7c8ccccc8c8c(-c9ccccc9)cccc87)ccc65)cc43)cc2)c([2H])c1[2H]]

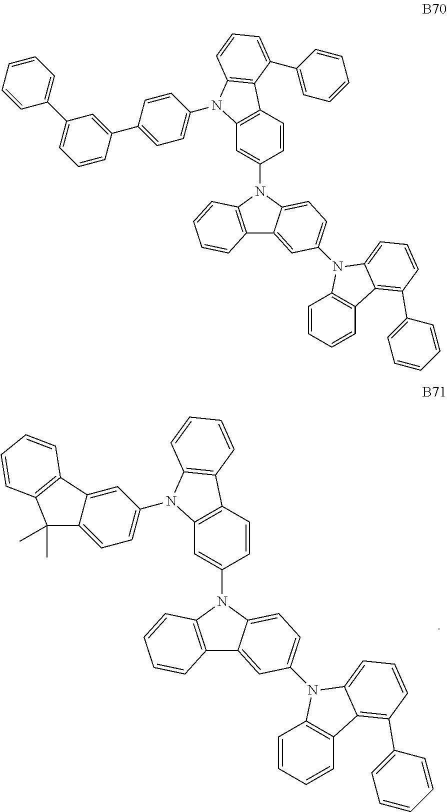 [SMILES: c1ccc(-c2cccc(-c3ccc(-n4c5cc(-n6c7ccccc7c7cc(-n8c9ccccc9c9c(-c%10ccccc%10)cccc98)ccc76)ccc5c5c(-c6ccccc6)cccc54)cc3)c2)cc1 ; CC1(C)c2ccccc2-c2cc(-n3c4ccccc4c4ccc(-n5c6ccccc6c6cc(-n7c8ccccc8c8c(-c9ccccc9)cccc87)ccc65)cc43)ccc21]

10. An organic electroluminescent device, comprising an anode and a cathode which are oppositely disposed, and a functional layer disposed between the anode and the cathode, wherein
the functional layer comprises the nitrogen-containing compound according to claim 1.

11. An electronic apparatus, comprising the organic electroluminescent device according to claim 10.

12. The nitrogen-containing compound according to claim 3, wherein substituent(s) of L are the same or different, and are respectively and independently selected from a deuterium, a phenyl or a pentadeuterophenyl.

13. The nitrogen-containing compound according to claim 4, wherein L is selected from the group consisting of:

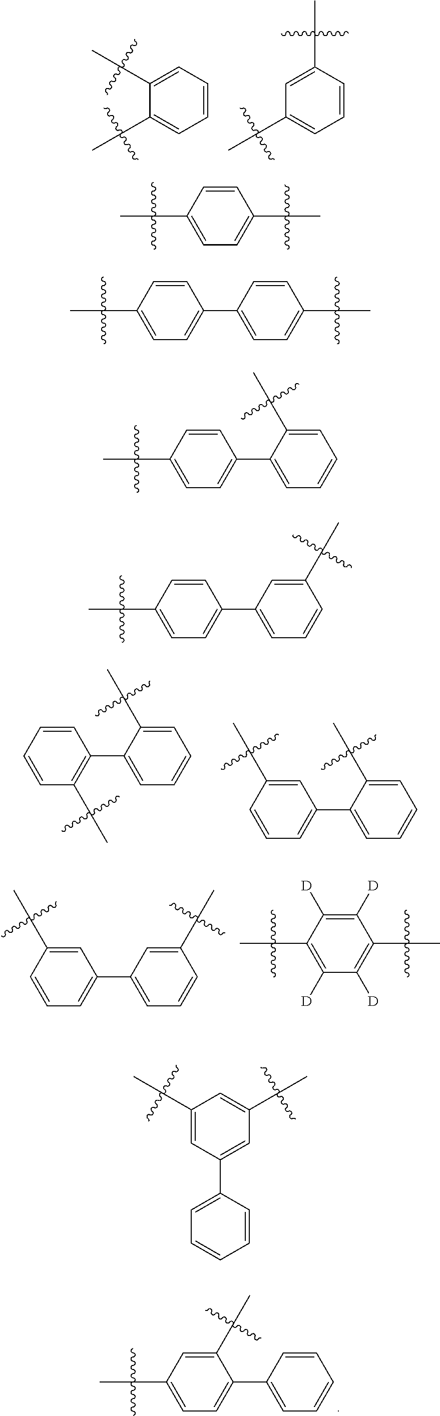

14. The nitrogen-containing compound according to claim 5, wherein substituent(s) of Ar₁ are the same or different, and are respectively and independently selected from a deuterium, a fluorine, a cyano, a methyl, an ethyl, a n-propyl, an isopropyl, a tert-butyl, a phenyl or a pentadeuterophenyl.

15. The nitrogen-containing compound according to claim 6, wherein Ar₁ is selected from the group consisting of:

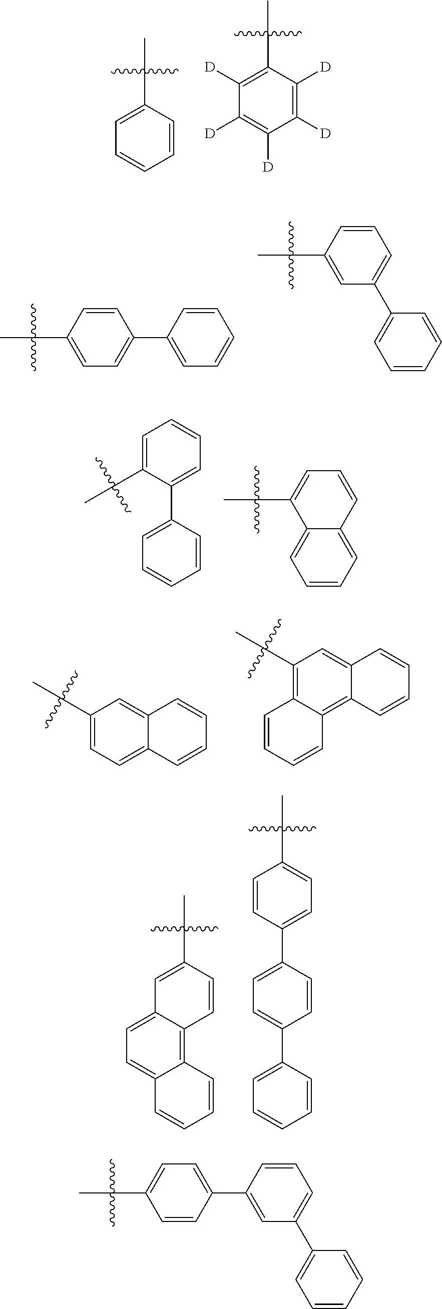

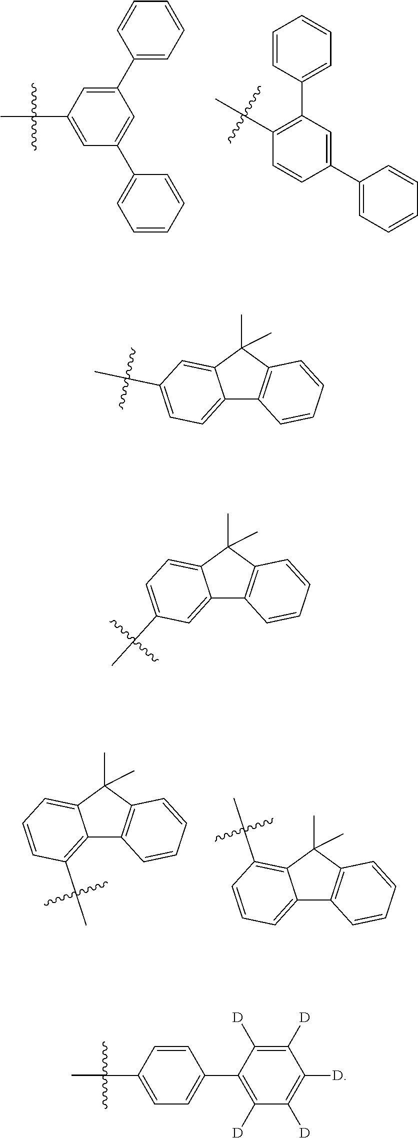

16. The organic electroluminescent device according to claim 10, wherein the functional layer comprises an organic light-emitting layer comprising the nitrogen-containing compound according to claim 1.
